SEQUENCE LISTING (2 sequences)

SEQ ID NO: 1 - DNA, 1700 bp, Homo sapiens
  CDS (275)...(895)
cttgcctgcaaacctttacttctgaaatgacttccacggctgggacgggaaccttccacccacagctatgcctctgattggtgaatggtgaaggtgcctgtctaacttttctgtaaaaagaaccagctgcctccaggcagccagccctcaagcatcacttacaggaccagagggacaagacatgactgtgatgaggagctgctttcgccaatttaacaccaagaagaattgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtctagaccaggacctccctccccctggcactggtttgttccctgtgtcatttcaaacagtctcccttcctatgctgttcactggacacttcacgcccttggccatgggtcccattcttggcccaggattattgtcaaagaagtcattctttaagcagcgccagtgacagtcagggaaggtgcctctggatgctgtgaagagtctacagagaagattcttgtatttattacaactctatttaattaatgtcagtatttcaactgaagttctatttatttgtgagactgtaagttacatgaaggcagcagaatattgtgccccatgcttctttacccctcacaatccttgccacagtgtggggcagtggatgggtgcttagtaagtacttaataaactgtggtgctttttttggcctgtctttggattgttaaaaaacagagagggatgcttggatgtaaaactgaacttcagagcatgaaaatcacactgtctgctgatatctgcagggacagagcattggggtgggggtaaggtgcatctgtttgaaaagtaaacgataaaatgtggattaaagtgcccagcacaaagcagatcctcaataaacatttcatttcccacccacactcgccagctcaccccatcatccctttcccttggtgccctccttttttttttatcctagtcattcttccctaatcttccacttgagtgtcaagctgaccttgctgatggtgacattgcacctggatgtactatccaatctgtgatgacattccctgctaataaaagacaacataactca

SEQ ID NO: 2 - protein, 206 aa, Homo sapiens
MNFQQRLQSLWTLARPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

(19) United States
(12) Patent Application Publication
(10) Pub. No.: US 2006/0110376 A1
(43) Pub. Date: May 25, 2006

(54) MDA-7 and free radicals in the treatment of cancer

(76) Inventors: Paul B. Fisher, Scarsdale, NY (US); Rahul V. Gopalkrishnan, New York, NY (US); Irina Lebedeva, Bronx, NY (US); Steven Grant, Richmond, VA (US); Adly Yacoub, Richmond, VA (US); Paul Dent, Glen Allen, VA (US)

Correspondence Address: BAKER & BOTTS, 30 ROCKEFELLER PLAZA, NEW YORK, NY, 10112, US

(21) Appl. No.: 11/158,743

(22) Filed: Jun. 22, 2005

Related U.S. Application Data
(63) Continuation of application No. PCT/US03/28512, filed on Sep. 9, 2003.
(60) Provisional application No. 60/486,870, filed on Jul. 10, 2003.
(60) Provisional application No. 60/486,533, filed on Jul. 10, 2003.
(60) Provisional application No. 60/436,281, filed on Dec. 23, 2002.
(60) Provisional application No. 60/436,273, filed on Dec. 23, 2002.

(51) Int. Cl. A61K 48/00

(52) U.S. Cl. 424/93.21; 514/44

(57) ABSTRACT

The present invention relates to methods of treating a cancer in a subject comprising generating within one or more cancer cells of a subject an effective amount of MDA-7 and an effective amount of one or more free radicals. The present invention further relates to methods of inhibiting proliferation or promoting death in a cancer cell of a subject comprising generating within one or more cancer cells of a subject an effective amount of MDA-7 and an effective amount of one or more free radicals. Generation of an effective amount of MDA-7 can occur by administering to the cancer cell an effective amount of an mda-7 nucleic acid, MDA-7 protein, functional equivalents of either of these molecules, by upregulation of the endogenous mda-7 gene, or by stabilization of the mda-7 mRNA. Generation of one or more free radicals in a cancer cell can occur by exposing the cancer cell to an effective amount of ionizing radiation, a free radical, a generator of a free radical, a ROS, a generator of a ROS, or a disruptor of mitochondrial membrane potential.

### CROSS REFERENCE TO RELATED APPLICATIONS

This application claims priority to U.S. Provisional Patent Appl. Ser. No. 60/436,273, filed Dec. 23, 2002; to U.S. Provisional Patent Appl. Ser. No. 60/436,281, filed Dec. 23, 2002; to U.S. Provisional Patent Appl. Ser. No. 60/486,533, filed Jul. 10, 2003; and to U.S. Provisional Patent Appl. Ser. No. 60/486,870, filed Jul. 10, 2003; the contents of which are incorporated herein in their entireties.

### STATEMENT REGARDING FEDERALLY SPONSORED RESEARCH

The subject matter described herein was supported in part by National Institutes of Health Grants 5R01CA035675, 5R01CA088906, 1R01CA097318, 5R01DK052825, and 5P01NS031492, and by Department of Defense Grants BC98-0148 and DAMD17-98-1-8053, so that the United States Government has certain rights herein.

### 1. INTRODUCTION

The present invention relates to methods of treating a cancer and/or tumor in a subject comprising generating within one or more cancer cells of a subject an effective amount of MDA-7 and an effective amount of one or more free radicals. The present invention further relates to methods of inhibiting proliferation or promoting death in a cancer cell of a subject comprising generating within one or more cancer cells of a subject an effective amount of MDA-7 and an effective amount of one or more free radicals. Generation of an effective amount of MDA-7 can occur by administering to the cancer cell an effective amount of a nucleic acid encoding MDA-7, an isolated and purified MDA-7 protein, or functional equivalents thereof. Generation of an effective amount of MDA-7 within the cell also may occur by upregulating expression of the mda-7 gene or by stabilizing mda-7 mRNA levels within the cell. Generation of one or more free radicals in a cancer cell can occur by exposing the cancer cell to an effective amount of ionizing radiation, a free radical, a generator of a free radical, a reactive oxygen species (ROS), a generator of a ROS, or a disruptor of mitochondrial membrane potential.

### 2. BACKGROUND OF THE INVENTION

### 2.1. Differentiation Therapy

Aberrant growth and differentiation are properties frequently observed in cancer cells (Sachs, 1978, Nature 274:535-9; Scott 1997, Pharmacol. Ther. 73:51-65; Leszczyniecka et al., 2001, Pharmacol. Ther. 90:105-156). In these contexts, developing strategies to re-program tumor cells to undergo irreversible growth arrest and terminal differentiation, a process termed ‘differentiation therapy,’ provides unique opportunities for therapeutic intervention (Sachs, 1978, Nature 274:535-9; Scott 1997, Pharmacol. Ther. 73:51-65; Leszczyniecka et al., 2001, Pharmacol. Ther. 90:105-156). The basic premise underlying differentiation therapy is that tumor cells either fail to produce or make subthreshold levels of gene products essential for maintaining growth control and normal programs of differentiation (Sachs, 1978, Nature 274:535-9; Fisher et al., 1985, J. Interferon Res. 5:11-22; Jiang et al., 1993, Mol. Cell. Different. 1:41-66; Jiang et al., 1994, Mol. Cell. Different. 2:221-39; Scott, 1997, Pharmacol. Ther. 73:51-65; Leszczyniecka et al., 2001, Pharmacol. Ther. 90:105-156).

This hypothesis has been tested in the context of human melanoma cells, which can be induced to irreversibly growth arrest and terminally differentiate by treatment with fibroblast interferon (IFN-β) and the protein kinase C activator mezerein (MEZ) (Jiang and Fisher, 1993, Mol. Cell. Different. 1:285-299; Jiang et al, 1993, Mol. Cell. Different. 1:41-66; Jiang et al., 1994, Mol. Cell. Different. 2:221-39). To identify genes expressed specifically during terminal differentiation, HO-1 human melanoma cells were terminally differentiated by treatment with IFN-β+MEZ (Fisher et al., 1985, J. Interferon Res. 5:11-22) and temporally spaced mRNAs were collected and used to generate a cDNA library (Jiang and Fisher, 1993, Mol. Cell. Different. 1:285-299). A similar temporal cDNA library was prepared from actively proliferating HO-1 cells not induced to growth arrest and terminally differentiate. These two cDNA libraries were subtracted (differentiated minus control) resulting in the construction of a temporally spaced subtracted (TSS) cDNA library theoretically enriched for genes modified during HO-1 terminal differentiation (Jiang and Fisher, 1993, Mol. Cell. Different. 1:285-299). By using various screening methodologies, including random clonal isolation and Northern hybridization (Jiang and Fisher, 1993, Mol. Cell. Different. 1:285-299), reverse Northern hybridization of arrayed cDNA clones (Huang et al., 1999, Gene 236:125-131) and high-density microarray analyses of cDNA clones (Huang et al., 1999, Oncogene 18:3546-52), a spectrum of genes associated with and potentially causative of melanoma growth arrest and differentiation have been isolated (Jiang and Fisher, 1993, Mol. Cell. Different. 1:285-299; Huang et al., 1999, Gene 236:125-131; Huang et al., 1999, Oncogene 18:3546-52). Among these genes was melanoma differentiation associated gene-7, hereafter “mda-7.” Subsequent studies have identified mda-7 as a member of the IL-10 family of cytokines and it has been designated as IL-24 (Wang et al., 2002, J. Biol. Chem. 277:7341-7347).

### 2.2. Identification and Initial Characterization of MDA-7/IL-24

Using differentiation induction subtraction hybridization (DISH) (Jiang et al., 1993, Mol. Cell. Different. 1:41-66; Huang et al., 1999, Gene 236:125-131), mda-7/IL-24 was identified as a gene displaying no or minimal RNA expression in actively proliferating melanoma cells, with elevated de novo expression in normal melanocytes and inducible expression in terminally differentiated melanoma cells (Jiang et al., 1995, Oncogene 11:2477-2486; WO95/11986). Initial characterization of the mda-7/IL-24 cDNA indicated that it encoded a protein of 23.8 kDa (Jiang et al., 1995, Oncogene 11:2477-2486), and that this protein contained a small stretch of sequence homology to IL-10 (54% in 42 amino acids). Southern blot analysis documented that mda-7/IL-24 is an evolutionarily-conserved gene with homologous sequences in the genomic DNAs of yeast, simian, bovine, canine and feline origin (Jiang et al., 1995, Oncogene 11:2477-2486). Expression analysis in HO-1 cells indicated lack of induction by IFN-β, a small induction by MEZ and during serum starvation, and maximum induction following treatment with IFN-β+MEZ (Jiang et al., 1995, Oncogene 11:2477-2486). These studies also documented that mda-7/IL-24 mRNA expression inversely correlated with melanoma progression from melanocyte to metastatic melanoma in clinical patient-derived specimens (Jiang et al., 1995, Oncogene 11:2477-2486). Transfection of C8161 metastatic human melanoma cells with an expression construct encoding mda-7/IL-24 reduced colony formation (Jiang et al., 1995, Oncogene 11:2477-2486), and using an HO-1 cell line containing an mda-7/IL-24 gene regulated by dexamethasone through a mouse mammary tumor virus promoter, expression of mda-7/IL-24 was growth suppressive (Jiang et al., 1995, Oncogene 11:2477-2486).

### 2.3. MDA-7/IL-24: A Broad Spectrum Cancer-Specific Apoptosis-Inducing Gene

mda-7/IL-24 has been found to reduce colony formation in a broad spectrum of human tumor cells irrespective of the status of their p53, Rb, Bax or p16 genes, including osteosarcoma and carcinomas of the breast, cervix, colon, nasopharynx and prostate (Jiang et al., 1996, Proc. Natl. Acad. Sci. U.S.A. 93:9160-9165; U.S. Pat. No. 5,710,137). In contrast, mda-7/IL-24 did not significantly alter growth in normal early passage human mammary breast epithelial cells, the HBL-100 normal breast epithelial cell line or early passage human skin fibroblasts (Jiang et al., 1996, Proc. Natl. Acad. Sci. U.S.A. 93:9160-9165). These studies demonstrate that mda-7/IL-24 has cancer-specific growth suppressing properties in a broad range of human tumor cell types with diverse genetic alterations.

As an approach to more efficiently administer mda-7/IL-24 and to begin to define the mechanism by which mda-7/IL-24 selectively affects cancer cell proliferation, a replication-incompetent adenovirus (Ad.mda-7) was constructed (Su et al., 1998, Proc. Natl. Acad. Sci. U.S.A. 95:14400-14405). Studies in the context of breast carcinoma cells demonstrated that Ad.mda-7 selectively induced growth suppression and this process occurred by induction of programmed cell death (apoptosis) (Su et al., 1998, Proc. Natl. Acad. Sci. U.S.A. 95:14400-14405). In contrast, as observed with transfection, infection of normal mammary epithelial and HBL-100 cells with Ad.mda-7 did not significantly affect growth or reduce viability. Analysis of the potential mechanism by which mda-7/IL-24 induced apoptosis indicated up-regulation of the pro-apoptotic molecule Bax uniquely in breast cancer cells, irrespective of their p53 gene status. Additionally, the level of the pro-apoptotic protein Bcl-2 was reduced in multiple breast carcinoma cells following Ad.mda-7 infection.

Infection of an expansive array of cancer and normal cell types with Ad.mda-7 demonstrates that mda-7/IL-24 has wide-ranging cancer-specific apoptosis promoting activity (Su et al., 1998, Proc. Natl. Acad. Sci. U.S.A. 95:14400-14405; Madireddi et al., 2000, Adv. Exp. Med. Biol. 465:239-261; Saeki et al., 2000, Gene Ther. 7:2051-2057; Huang et al., 2001, Oncogene 20:7051-63; Mhashilkar et al., 2001, Mol. Med. 7:271-282; Cao et al., 2002, Mol. Med. 8:869-876; Kawabe et al., 2002, Mol. Ther. 6:637-644; Lebedeva et al., 2002, Oncogene 21:708-718; Pataer et al., 2002, Cancer Res. 62:2239-2243; Saeki et al., 2002, Oncogene 21:4558-4566; Sarkar et al., 2002, Proc. Natl. Acad. Sci. U.S.A. 99:10054-10059; Su et al., 2001, Proc. Natl. Acad. Sci. U.S.A. 98:10332-10337; Pataer et al., 2003, J. Thorac. Cardiovasc. Surg. 125:1328-1335; Sauane et al., 2003, Cytokine Growth Factor Rev. 14:35-51; Sauane et al., 2003, J. Cell. Physiol. 196:334-345; Su et al., 2003 Oncogene 22:1164-1180; Yacoub et al., 2003, Mol. Cancer Therapeut. 2:623-632). Although the mechanism underlying the differential pro-apoptotic activity of mda-7/IL-24 toward cancer versus normal cells is not currently known, this cancer-selective activity in most cases appears not to be a consequence of differences in mda-7 expression, protein production or secretion following infection with Ad.mda-7 (Mhashilkar et al., 2001, Mol. Med. 7:271-282; Lebedeva et al., 2002, Oncogene 21:708-718; Su et al., 2003 Oncogene 22:1164-1180). In specific cell types, including breast, pancreatic and prostate carcinomas, melanomas and malignant gliomas, induction of apoptosis correlates with changes in the ratio of pro-apoptotic proteins (such as Bax and Bak) to anti-apoptotic proteins (such as Bcl-2 and Bcl-xL), thereby shifting the balance from survival to programmed cell death (Saeki et al., 2000, Gene Ther. 7:2051-2057; Lebedeva et al., 2002, Oncogene 21:708-718; Su et al., 2003 Oncogene 22:1164-1180). Changes in cell cycle are also evident in some, but not all, cancer cells infected with Ad.mda-7 (Saeki et al., 2000, Gene Ther. 7:2051-2057; Lebedeva et al., 2002, Oncogene 21:708-718; Su et al., 2003 Oncogene 22:1164-1180). A cell cycle change seen in Ad.mda-7-infected melanomas, non-small cell lung carcinomas, prostate carcinomas and certain malignant gliomas is an increase in the proportion of cells in the G2/M phase (Saeki et al., 2000, Gene Ther. 7:2051-2057; Lebedeva et al., 2002, Oncogene 21:708-718; Su et al., 2003 Oncogene 22:1164-1180). Apoptosis induction associates with activation of the caspase cascade in specific tumor systems, including activation of caspase-9 and caspase-3 and cleavage of PARP, a caspase substrate (Saeki et al., 2000, Gene Ther. 7:2051-2057; Mhashilkar et al., 2001, Mol. Med. 7:271-282; Pataer et al., 2002, Cancer Res. 62:2239-2243).

The present invention relates to methods of enhancing the ability of mda-7 and its encoded protein to inhibit malignant cell growth and proliferation and to promote apoptosis. The present invention provides a method for the treatment of cancer in a subject comprising administering mda-7 nucleic acid or MDA-7 protein in combination with radiation therapy and/or one or more sources of free radicals, including free radicals, generators of free radicals, reactive oxygen species (ROS), generators of reactive oxygen species (ROS), and/or disruptors of mitochondrial membrane potential. This invention is based, at least in part, on the observation that the ability of mda-7/IL-24 to induce apoptosis and reduce clonogenic survival can be augmented in malignant glioma, mammary, prostate, renal, lung and other cancer cells by agents that generate free radicals. While Kawabe et al. reported that the pro-apoptotic effects of Ad.mda-7 in non-small cell lung cancer cells could be augmented by radiation therapy (Kawabe et al., 2002, Mol. Ther. 6:637-644), this reference does not disclose the use of radiation to enhance mda-7 nucleic acid-mediated cell death in other forms of cancer, the use of exogenously-administered MDA-7 protein in conjunction with radiation, nor the use of free radicals or free radical generators other than radiation in conjunction with mda-7 therapy.

### 3. SUMMARY OF THE INVENTION

The present invention relates to methods of treating a cancer in a subject comprising generating, within one or more cancer cells of a subject, an effective amount of mda-7 nucleic acid, MDA-7 protein, or functional equivalents thereof, and generating within the same cancer cells an effective amount of one or more species of free radicals. The invention is based, at least in part, on the observations that the dose-dependent growth suppression and apoptosis induced in cultured human cancer cells but not normal cultured human cells by administration of either mda-7 nucleic acid or purified GST-MDA-7 protein could be significantly potentiated by the prior, concurrent, or subsequent administration of ionizing radiation, free radical generators such as arsenic trioxide, NSC656240 or N-(4-hydroxyphenyl) retinamide (4-HPR), or mitochondrial membrane potential disruptors such as the peripheral benzodiazapine receptor agonist PK11195, and that this MDA-7-mediated cytotoxicity could largely be prevented by the administration of either the anti-oxidants N-acetyl-cysteine (NAC) and Tiron or the mitochondrial membrane permeability inhibitors cyclosporine (CsA) or bongkrekic acid (BA). The present invention exhibits a significant advantage over previous approaches in that the combination of mda-7 nucleic acid or MDA-7 protein with ionizing radiation, free radicals, generators of free radicals, ROS, generators of ROS, or disruptors of mitochondrial membrane potential, or various combinations thereof is selectively toxic to human cancer cell lines.

### 4. BRIEF DESCRIPTION OF THE FIGURES

FIG. 1A-F. Effect of Ad.vec, Ad.mda-7 and Ad.wtp53 on the growth of normal (PHFA) and immortal (PHFA-Im) human fetal astrocytes and mutp53 and wtp53 malignant gliomas. The various cell lines were uninfected (Control) or infected with 100 pfu/cell of the indicated virus and cell growth was determined by hemocytometer over an 8-day period. Results are expressed as the average of triplicate samples±S.D. Replicate experiments varied by ≦12%.

FIG. 2A-F. Temporal effects on mda-7 mRNA expression in normal and immortal human fetal astrocytes and malignant gliomas after infection with Ad.mda-7. The indicated cell type was infected with 100 pfu/cell of Ad.mda-7 and total RNA was isolated at the times indicated and analyzed by Northern blotting. Ten μg of each RNA sample was analyzed by Northern blotting. Blots were probed with a random-primed [³²P]-labeled mda-7 cDNA, the blots were stripped and then reprobed with a random-primed [³²P]-labeled gapdh cDNA probe. Blots were exposed for autoradiography. (A) PHFA; (B) PHFA-Im; (C) U87MG; (D) U25MG; (E) U373MG; (F) T98G.

FIG. 3. Determination of intracellular and secreted MDA-7 protein in immortal human fetal astrocytes and malignant gliomas after infection with Ad mda-7. Normal immortalized primary human fetal astrocytes (PHFA-Im) and malignant gliomas (U87MG, U251MG and T98G) were untreated (Control) or infected with 100 pfu/cell of Ad.vec and Ad.mda-7, and 24 and 48 hpi supernatants and 24, 48 and 72 hpi cell lysates were collected and levels of MDA-7 protein were determined by Western blotting. A total of 25 μl of supernatant and 50 μg of cell lysates were used for Western blotting assays. Arrows on the left indicate secreted MDA-7 protein and brackets and arrows on the right indicate multiple sized MDA-7 proteins in cell lysates.

FIG. 4. Production of wtp53 protein in PHFA-Im and malignant gliomas following infection with Ad.wtp53. Cells were untreated (Control) or infected with 100 pfu/cell of Ad.vec or Ad.wtp53 and protein samples were collected in RIPA buffer at different time points. Samples (30 μg of total protein) were run on 12% SDS PAGE, transferred to Immobilon P PVDF membranes and stained with anti p53 monoclonal antibody.

FIG. 5. Induction of early and late apoptosis and necrosis by Ad.mda-7 and Ad.wtp53 in malignant gliomas as monitored by Annexin V binding. The indicated cells were untreated (control) or infected with 100 pfu/cell of Ad.vec, Ad.mda-7 or Ad.wtp53. Cells were stained 30 h later with FITC labeled Annexin V and PI and immediately analyzed by flow cytometry. The percentage of early apoptotic cells (only Annexin V stained) and late apoptotic and necrotic cells (stained with both Annexin V and P1) was calculated using CellQuest software (Becton Dickinson, San Jose, Calif.).

FIG. 6. Induction of apoptosis as monitored by A₀ DNA content by Ad.mda-7 and Ad.wtp53 in malignant gliomas. The indicated cells were untreated (control) or infected with 100 pfu/cell of Ad.vec, Ad.mda-7 or Ad.wtp53 and harvested at 24, 48 and 72 hpi, fixed and stained with PI as described in Materials and methods. The percentage of the cells in A₀ fraction was calculated using the CellQuest software (Becton Dickinson).

FIG. 7. Cell cycle changes in malignant gliomas following infection with Ad.mda-7 and Ad.wtp53. Cells were untreated (control) or infected with 100 pfu/cell of Ad.vec, Ad.mda-7 or Ad.wtp53, harvested at 24, 48 and 72 hpi, fixed and stained with PI. Viable, non-apoptotic cells were gated using CellQuest software, and cell cycle phase distribution of the cells was determined for each cell type. The percentage of cells in G₂/M phase after Ad.vec, Ad.mda-7 (Panel A) or Ad.wtp53 (Panel B) infection was determined.

FIG. 8. Determination of Bcl-2, Bcl-XL, BAX, BAK and EF-1α protein levels in normal immortal fetal astrocytes and malignant gliomas following infection with Ad.mda-7 or Ad.wtp53. Cells were untreated (control) or infected with 100 pfu/cell of Ad.vec, Ad.mda-7 or Ad.wtp53 and protein lysates were prepared at the specified time points. Samples of 50 μg of total protein were run on 12% SDS-PAGE, transferred to a PVDF membrane and stained with different antibodies. Bcl-family protein expression, as determined by Western blot analysis, was quantitatively analyzed via laser-scanning densitometry using NIH Image Version 1.61 software.

FIG. 9. Induction of GADD family genes following infection with Ad.mda-7 and Ad.wtp53 in malignant gliomas. U87MG, PHFA-Im and U251MG malignant gliomas were infected with either Ad.vec, Ad.mda-7 or Ad.wtp53 at an M.O.I. of 100 pfu/cell for three days. Total RNA was extracted and the expression profiles of GADD153, GADD45α, GADD34 and GAPDH mRNAs were determined by Northern blot analysis.

FIG. 10A-B. Ad.mda-7 infection sensitizes malignant gliomas to radiation-induced growth suppression and induction of apoptosis. U87MG and U251MG cells were plated and 24 h later were infected with Ad.mda-7 or Ad.vec at an m.o.i. of ˜50 pfu/cell. Cells were cultured for an additional 24 h prior to irradiation. Cultures were then irradiated (6 Gy) and the growth of cells determined 4 days later (5 days after infection) using MTT assays. Parallel experiments examined the amount of apoptosis 4 days after exposure. Panel A. The combination of Ad.mda-7 and ionizing radiation caused a statistically significant additional decrease in growth potential of U87MG and U251MG glioma cells. * p<0.05 less than Ad.vec alone; # p<0.05 less than either radiation or Ad.mda-7 alone. Panel B. The combination of mda-7 and ionizing radiation caused a statistically significant additional increase the numbers of apoptotic cells, as judged by Wright Giemsa staining for apoptotic cell morphology. % p<0.05 greater than either radiation or Ad.mda-7 alone.

FIG. 11. Purified GST-MDA-7 protein sensitizes malignant glioma cells to radiation-induced growth suppression. U87MG and U251MG cells were plated and 24 h later were incubated with GST-MDA-7 or control (GST) at a final concentration of 0.25 μg/ml. Cells were cultured for an additional 30 min prior to irradiation. Cells were then irradiated (6 Gy) and the growth of cells determined 4 days later (5 days after infection) using MTT assays. The panel shows that the combination of GST-MDA-7 and ionizing radiation caused a statistically significant additional decrease in growth potential of U87MG and U251MG glioma cells. * p<0.05 less than Ad.vec alone; # p<0.05 less than either radiation or Ad.mda-7 alone.

FIG. 12. The combination of Ad.mda-7 and irradiation synergistically induce GADD153 expression in mutp53 and wtp53 gliomas. U251MG (mutp53) and U87G (wtp53) gliomas were infected with Ad.mda-7 or Ad.vec and irradiated as described in FIG. 10. The cells were harvested day 1 to day 3 post-irradiation, total RNA was extracted and the expression of GADD153 and GAPDH mRNAs were analyzed by Northern blotting analysis.

FIG. 13A-D. Ad.mda-7 suppresses glioma cell growth and enhances radiosensitivity. Glioma cells were cultured for 24 h after plating then infected with Ad.mda-7 or CMV control viruses at the following multiplicities of infection: Panel A. RT2 cells, 5 m.o.i.; Panel B. RT2 cells, 25 m.o.i.; Panel C. RT2 cells, 50 m.o.i.; Panel D. primary rodent astrocytes, 50 m.o.i. The cells were irradiated, as indicated, 24 hours after infection. MTT assays were performed 4 days post radiation. The values were normalized to the control unirradiated cells which is defined as 1.00. Data are the means of 12 data points±SEM from a representative experiment (n=3, * p<0.05 less than corresponding control value when corrected for the growth suppressive effects of Ad.mda-7 alone).

FIG. 14. GST-MDA-7 reduces the proliferation of glioma cells and enhances radiation-induced cell killing. Cells were cultured for 24 h then treated with GST-MDA-7 or GST at the concentrations indicated. As indicated, 24 h after GST-MDA-7 treatment, cells were irradiated (6 Gy). Cells were isolated 96 h after irradiation and cell numbers and viability determined by trypan blue exclusion staining and by Wright Giemsa staining of fixed cells. In parallel, cell numbers were also determined 96 h after irradiation by MTT assay. Panel A. GST-MDA-7 inhibits the proliferation of RT2 cells in a dose-dependent fashion and enhances apoptotic cell death as judged by Giemsa staining for nuclear DNA fragmentation. Panel B. GST-MDA-7 (0.5 nM) interacts with radiation in a greater than additive fashion to suppress RT2 cell growth in MTT assays. Panel C. Left set of bars: GST-MDA-7 (5.0 nM) interacts with radiation in a greater than additive fashion to enhance apoptotic cell killing as judged by Giemsa staining. Right set of bars: GST-MDA-7 (5.0 TIM) interacts with radiation in a greater than additive fashion to enhance apoptotic cell killing as judged by trypan blue exclusion staining. Data are the means±SEM of 3 separate experiments # p<0.05 greater than control infected cells; * p<0.05 less than control infected cells; % p<0.05 less than control infected cells corrected for the anti-proliferative effects of GST-MDA-7.

FIG. 15A-D. Ad.mda-7 reduces the expression of Bcl-_{XL} and increases the levels of BAX, consistent with a causal role of Bcl-_{XL} expression in the radiosensitizing effect. Cells were cultured for 24 h after plating then infected with Ad.mda-7 or CMV control viruses (25 m.o.i.). The cells were irradiated (6 Gy) 24 hours after infection. Cells were isolated 96 h after irradiation and processed. Panel A. Ad.mda-7 and radiation suppress the expression of Bcl-_{XL} and enhance BAX levels in RT2 cells 96 h after irradiation. Cells were processed for SDS PAGE and immunoblotting. A representative study (n=3). Panel B. RT2 cells were cultured for 24 h then infected with Ad.mda-7 or CMV control viruses at 25 m.o.i. The cells were irradiated (6 Gy) 24 hours after infection. Cells were isolated 96 h after irradiation and the integrity of nuclear DNA under each condition determined. Data are from two representative experiments. Panel C. Over-expression of Bcl-_{XL} protects RT2 cells from the growth inhibitory effects of Ad.mda-7. Cells were cultured for 24 h after plating then infected with Ad.Bcl-_{XL}, Ad.mda-7 or CMV control viruses at 25 m.o.i. The cells were irradiated (6 Gy) 24 hours after infection. Cells were processed 96 h after irradiation via MTT assay to determine cell numbers. Data are the means±SEM of 3 separate experiments. Panel D. Over-expression of Bcl-_{XL} protects RT2 cells from the cytotoxic effects of Ad.mda-7. Cells were cultured for 24 h after plating then infected with Ad.Bcl-_{XL}, Ad.mda-7 or CMV control viruses at 25 m.o.i. The cells were irradiated (6 Gy) 24 hours after infection. Cells were processed 96 h after irradiation and cell viability determined via trypan blue exclusion assay. Data are the means±SEM of 3 separate experiments # p<0.05 greater than control infected cells.

FIG. 16A-B. Free radical scavengers N-acetyl-L-cysteine (NAC) abrogate Ad.mda-7 radiosensitization. Cells were cultured for 24 h then infected with Ad.mda-7 or CMV control viruses (25 m.o.i.). Cells were incubated for a further 24 h and then thirty minutes prior to irradiation (6 Gy), cells were treated with either vehicle (media) or 10 mM N-acetyl cysteine (NAC). Cells were isolated 96 h after irradiation and processed for either MTT assays or cell viability via trypan blue exclusion. Panel A. Radiation and Ad.mda-7 suppress RT2 cell growth: NAC abolishes the radiation-dependent enhancement in growth suppression. Panel B. Radiation and Ad.mda-7 enhance R T2 cell killing: NAC abolishes the radiation-dependent enhancement of cell death. Data are the means±SEM of 3 separate experiments # p<0.05 greater than control infected cells, ** p<0.05 less than corresponding value in cells not incubated with NAC.

FIG. 17. Ad.mda-7 prolongs animal survival and radiosensitizes RT2 cells in vivo. Cells were cultured for 24 h after plating then infected with Ad.mda-7 or CMV control viruses (25 m.o.i.). Fischer 344 rats were implanted intra-cranially with infected RT2 cells and 4 days after implantation, the head of each animal was irradiated. Animal survival was noted on a daily basis. Data are the total from 4 separate experiments of 4 animals per condition per experiment. Statistical analyses were performed using the log rank test. ** p<0.05 greater than unirradiated animals; # p<0.01 greater than Ad.mda-7 alone animals.

FIG. 18A-B. Expression of MDA-7 and β-galactosidase in RT2 cells. RT2 cells were cultured for 24 h, then infected with Ad.mda-7, Ad.β-galactosidase, or CMV control viruses (0-300 multiplicity of infection (m.o.i.), as indicated). Cells were harvested 48 hours after infection. Panel A. Protein samples were separated using SDS PAGE. The primary antibody was raised in a rabbit versus bacterially synthesized GST-MDA-7 fusion protein and immunoblotting performed using a purified IgG fraction. Data shown are from a representative experiment (n=3). Panel B. Cells (RT2, U251) were infected at the indicated m.o.i. Forty eight h after infection cells were fixed, and then stained with X-gal as described to determine the infection rate of RT2 and U251 cells. Data shown are ±SEM (n=3).

FIG. 19A-D. Ad.mda-7 suppresses glioma cell growth and enhances radiosensitivity. Glioma cells were cultured for 24 h after plating then infected with Ad.mda-7 or CMV control viruses at the following multiplicities of infection: Panel A. MTT assay, RT2 cells, 25 m.o.i. (38); Panel B. Colony formation assay, RT2 cells, 5 m.o.i. (33); Panel C. MTT assay, U251 cells, 25 m.o.i. Panel D. MTT assay, U373 cells, 25 m.o.i. The cells were irradiated, as indicated, 24 hours after infection. MTT and colony formation assays were performed. The values were normalized to the control unirradiated cells which is defined as 1.00. MTT data are the means of 12 data points±SEM from a representative experiment (n=3, * p<0.05 less than corresponding control value).

FIG. 20A-B. Ad.mda-7 causes an increase in glioma cell death that is enhanced in a greater than additive fashion by ionizing radiation. Cells were cultured for 24 h then infected with Ad.mda-7 or CMV control viruses (25 m.o.i.). The cells were irradiated (6 Gy) 24 hours after infection. Cells were isolated 96 h after irradiation and cell viability determined by trypan blue exclusion staining and by Wright Giemsa staining of fixed cells. Panel A. trypan blue staining RT2 cells, 25 m.o.i.; Panel B. expression and integrity of PARP and p32 pro-caspase 3 in RT2 cells, 25 m.o.i. Data are the means±SEM of 3-4 separate experiments #p<0.05 greater than control infected cells. Data are from a representative experiment (n=3).

FIG. 21A-B. The combination of Ad.mda-7 and radiation enhance RT2 cell numbers in G₁ phase and U251 cell numbers in G2/M phase: S phase cell numbers are reduced in both cell types. Glioma cells were cultured for 24 h then infected with Ad.mda-7 or CMV control viruses at 25 m.o.i. The cells were irradiated (6 Gy) 24 hours after infection. Cells were isolated 24 h after irradiation and the cell cycle distribution under each condition determined. Panel A. RT2 cell cycle distribution. Inset Panel. Expression of p21 and p53 in RT2 cells under each treatment condition. Panel B. U251 cell cycle distribution. Data are the means±SEM of three separate experiments.

FIG. 22A-E. MAPK and PI3K inhibitors sensitize cells to the toxic effects of Ad.mda-7. Cells were cultured for 24 h then infected with Ad.mda-7 or CMV control viruses (25 m.o.i.). Cells were incubated for an additional 24 h then treated with either 10 μm PD98059, 5 μm LY294002 or both drugs in combination: 30 min later the cells were irradiated (6 Gy). After irradiation (96 h), cells were harvested for processing. Panel A. Ad.mda-7 enhances p38 and ERK1/2 activity but not that of AKT or JNK1/2: radiation abolishes Ad.mda-7-induced ERK1/2 activity and enhances Ad.mda-7-dependent JNK1/2 activity. Immunoblotting data are from a representative experiment (n=3). Panel B. The anti-proliferative effect of Ad.mda-7 is enhanced by combined inhibition of MEK1/2 and PI3K. Data are the means±SEM of 3 experiments # p<0.05 less than control treated cells, * p<0.05 less than corresponding value in cells not infected with Ad-mda-7. Panel C. The toxicity of Ad.mda-7, as judged by Giemsa staining of fixed cells, is enhanced by combined inhibition of MEK1/2 and PI3K. Data are the means±SEM of 3 separate experiments. Panel D. The toxicity of Ad.mda-7, as judged by trypan blue exclusion staining of fixed cells, is enhanced by combined inhibition of MEK1/2 and PI3K. Data are the means±SEM of 3 separate experiments. Panel E. The JNK1/2 inhibitor SP600125 abolishes the radiosensitizing effect of Ad.mda-7. Cells were incubated with vehicle or SP600125 (10 μM) 30 min prior to irradiation and growth determined via MTT assay 4 days after irradiation (±SEM, n=4).

FIG. 23. Transfection of renal cell carcinoma cell lines with a plasmid to express MDA-7 results in reduced colony formation. Cells (2×10⁵) were plated in triplicate 60 mm dishes and cultured for 24 h prior to transfection with 1 μg of either control plasmid (pcDNA 3.1) or plasmid to express MDA-7 (pcDNA 3.1/mda-7). Twenty-four hours after transfection, cells were placed into selection media (400 μg/ml G418) and colony formation examined 14 days later, after fixing and crystal violet staining of colonies (±SEM n=3 * p<0.05 greater than pcDNA 3.1/mda-7 transfected cells).

FIG. 24. Coxsackievirus and adenovirus receptor expression is reduced in renal carcinoma cells compared to primary renal epithelial cells. Cells were plated in triplicate 60 mm dishes (˜0.2×10⁶) and 24 h later infected at the indicated multiplicity of infection (m.o.i.) with a recombinant type 5 adenovirus to express β-galactosidase. Forty-eight hours after infection, cells are fixed and processed to determine β-galactosidase expression. Panel A. β-galactosidase staining from a representative experiment. Panel B. Graphical data are the means of 3 experiments (±SEM).

FIG. 25A-D. GST-MDA-7 causes a dose-dependent reduction in the proliferation of renal carcinoma cells but not primary renal epithelial cells. Cells were plated in 12 well plates (˜1×10⁴ cells/well) and 24 h later treated with increasing concentrations of GST and GST-MDA-7 as indicated. Cell growth was determined via MTT assay 96 h after GST-MDA-7 treatment. Data are the means of 12 wells (one plate per condition, ±SEM) from a representative experiment using different preparations of GST-MDA-7 (n=3). Panel A. UOK121N cells. Panel B. A498 cells. Panel C. Primary renal epithelial cells. Panel D. Primary rat hepatocytes were isolated and cultured as described in Methods. Four hours after plating, hepatocytes were infected with either control CMV virus or Ad.mda-7 (at a multiplicity of infection of 50). Five days after infection, the media from the hepatocytes was removed and mixed with an equal volume of RCC line culture media. This media was added to cultures of A498 and UOK121N cells, plated in 12 well plates (˜1×10⁴ cells/well), 24 h previously. Cell growth was determined via MTT assay 96 h after MDA-7 treatment. Data are the means of 12 wells (one plate per condition, ±SEM, * p<0.05 less than control CMV media or fresh media) from a representative experiment using different hepatocyte infections (n=2).

FIG. 26. Arsenic trioxide causes a concentration-dependent reduction in primary and renal carcinoma cell growth. Cells were plated in 12 well plates (˜1×10⁴ cells/well) and 24 h later treated with increasing concentrations of As₂O₃ as indicated. Cell growth was determined via MTT assay 96 h after GST-MDA-7 treatment. Data are the means of 12 wells (one plate per condition, ±SEM) from a representative experiment using separate As₂O₃ formulations (n=3). Arsenic trioxide caused a dose-dependent enhancement in cell killing at higher concentrations >10 μM.

FIG. 27A-C. GST-MDA-7 and arsenic trioxide interact in a greater than additive fashion to reduce renal carcinoma cell growth. Cells were plated in 12 well plates (˜1×10⁴ cells/well) and 24 h later treated with increasing concentrations of GST, GST-MDA-7 and As₂O₃ (0.5 μM) as indicated. Cell growth was determined via MTT assay 96 h after GST-MDA-7 treatment. Data are the means of 12 wells (one plate per condition, ±SEM) from a representative experiment using different preparations of GST and GST-MDA-7 (n=3). Panel A. UOK121N cells. Panel B. A498 cells. Panel C. primary renal epithelial cells. * p<0.05 less than GST value cells; # p<0.05 less than corresponding GST-MDA-7 value without As₂O₃ co-treatment.

FIG. 28A-B. GST-MDA-7 and arsenic trioxide interact in a greater than additive fashion to enhance renal carcinoma killing that is blocked by the free radical scavenger N-acetyl cysteine. Cells were plated in 6 well plates (˜5×10⁴ cells/well) and 24 h later treated with GST, GST-MDA-7 (both 0.5 nM) and As₂O₃ (0.5 μM) as indicated. Where indicated, cells were pre-treated with 10 mM N-acetyl cysteine 1 h prior to addition of As₂O₃. Cells were isolated 96 h after GST-MDA-7 treatment. Cell death was determined on isolated fixed cells by Giemsa staining for apoptosis and necrosis (presented as a single value) as described (30). Panel A. UOK121N cells. Panel B. A498 cells. Data are the means of 6 wells (one plate per condition, ±SEM) from a representative experiment using different preparations of GST and GST-MDA-7 (n=3). * p<0.05 greater than GST value cells; # p<0.05 greater than corresponding GST-MDA-7 value without As₂O₃ co-treatment; $ P<0.05 less than value in cells not incubated with NAC.

FIG. 29A-C. GST-MDA-7 and arsenic trioxide interact to enhance cleavage of pro-caspase 3 and PARP in renal carcinoma cells that correlates with reduced expression of Bcl-x_{L} and enhanced activity of p38 and JNK1/2. Cells were plated in 100 mm plates (˜2×10⁵ cells/well) and 24 h later treated with GST, GST-MDA-7 (both 0.5 nM) and As₂O₃ (0.5 μM) as indicated. Cells were isolated 96 h after GST-MDA-7 treatment. Protein expression levels were determined using Bradford assay for total protein followed by SDS PAGE and immunoblotting. In parallel plates, nucleosomal DNA integrity was determined using agarose gel electrophoresis. Panel A. Expression of PARP, p32 pro-caspase 3, and Bcl-x_{L} in RCC lines. Panel B. Nucleosomal DNA integrity in RCC lines. Panel C. ERK1/2, JNK1/2 and p38 activity in RCC lines. Data are from a representative experiment (n=3).

FIG. 30A-B. GST-MDA-7 and arsenic trioxide interact in a greater than additive fashion to reduce renal carcinoma cell colony formation ability. Panel A. UOK121N cells. Panel B. A498 cells. Cells were plated in 6 well plates (˜5×10⁴ cells/well) and 24 h later treated with GST, GST-MDA-7 (both 0.5 nM) and As₂O₃ (0.5 μM) as indicated. Cells were isolated 96 h after GST-MDA-7 treatment and cell viability determined using trypan blue exclusion assay (see FIG. 28). Based on trypan blue negative viable cell values, 250, 500 and 2000 viable cells were re-plated in Linbro plates. The plating density for colony formation assays depended upon the prior treatment of the cells and data obtained in FIGS. 27 and 28. 10-14 days after plating, cells were fixed and stained with crystal violet. Colony formation was determined using visual counting, and a colony was defined as a group of >50 cells. Data are the means of 3 separate experiments (±SEM). * p<0.05 less than control cells; # p<0.05 less than As₂O₃ or GST-MDA-7 treated cells.

FIG. 31A-D. ROS induction correlates with Ad.mda-7-induced cell death in prostate cancer cells. Panel A. Ad.mda-7-induced cell death is inhibited by antioxidants. Cells were seeded in 96-well plates, pretreated with NAC (5 mM) or Tiron (1 mM) for 2 h and infected with Ad.mda-7. Forty eight hours later, viability was assessed by MTT assay. Panel B. ROS-producing substances enhance Ad.mda-7-induced cell death. Cells were seeded in 96-well plates, infected with Ad.mda-7 and treated with As₂O₃ (10 μM) or NSC656240 (400 nM). Forty eight h later, viability was assessed by MTT assay. Panel C. Treatment of prostate carcinoma cells with Ad.mda-7 induces ROS generation that is blocked by NAC and is enhanced by NSC656240 and As₂O₃ (ARS). After treatment, cells were stained using DCF-DA and analyzed using flow cytometry. Panel D. Antioxidant treatment blocks or significantly inhibits apoptosis induced by Ad.mda-7 in prostate cancer cells, while NSC656240 and As₂O₃ (ARS) treatment increases apoptosis induced by Ad.mda-7. After treatment, cells were washed and stained with Annexin V-FITC conjugate. Late apoptotic and necrotic cells were excluded using PI staining. Results are the mean of three independent experiments performed with triplicate samples±S.E.

FIG. 32. Kinetics of mitochondrial alteration, ROS generation and plasma membrane changes induced by Ad.mda-7 treatment of prostate cancer and immortalized normal cells. Cells were infected with Ad.vec or Ad.mda-7, and analyzed using flow cytometry at indicated times. Changes in the mitochondrial transmembrane potential Δψm (closed triangles) were measured with DiOC₆(3), ROS generation was measured using DCF-DA (open circles, hydrogen peroxide and NO). The percentage of apoptotic cells (hatched bars, right Y-axis) was measured using simultaneous staining with Annexin V-FITC conjugate and PI. Since the data for Ad.vec-infected cells were not significantly different from the data for control (untreated cells), Ad.mda-7-related data is presented in this figure. Results are from a single experiment with triplicate samples that varied by ≦10%. Qualitatively similar results were obtained in two additional experiments.

FIG. 33A-C. MPT is an early event in Ad.mda-7-induced cell death and apoptosis preceding caspase activation. Prostate cancer and normal immortalized P69 cells were pretreated with inhibitors of MPT CsA (100 nM) or BA (50 μM), with the enhancer of MPT PK11195 (50 μM) or with the pan-caspase inhibitor z-VAD.fmk (50 μM) for 2 h following infection with Ad.vec or Ad.mda-7. Cellular viability was assessed by MTT assay 48 h after infection (Panel A). Mitochondrial membrane potential (DiOC₆(3) staining, Panel B) and apoptotic changes (Annexin V staining, Panel C) were assessed 18 h (LNCaP cells) and 24 h (DU-145, PC-3 and P69 cells) after infection.

FIG. 34A-B Bcl-2 and Bcl-x_{L} overexpression differentially protects prostate cancer cells from Ad.mda-7-induced cell death and apoptosis by blocking MPT and subsequent ROS generation and a model for Ad.mda-7 induced changes in mitochondria culminating in apoptosis. Prostate cancer cells stably transfected with Bcl-2, Bcl-x_{L} or empty vector (neo) were infected with Ad.vec or Ad.mda-7 as described and mitochondrial membrane potential (DiOC₆(3) staining (Panel A) and ROS production (DCF-DA staining, Panel B) were assessed 18 h (LNCaP cells) and 24 h (DU-145, PC-3 and P69 cells) after infection.

FIG. 35. Effect of transduction by Ad.mda-7 on the growth of the ovarian cancer cell line SKOV3 in the presence or absence of N-(4-hydroxyphenyl) retinamide (4-HPR).

FIG. 36. Combined treatment of pancreatic carcinoma cells by NSC656240 and Ad.mda-7 causes cell death irrespective of K-ras status and does not affect the growth of normal cells.

FIG. 37. Combined treatment of pancreatic carcinoma cells by NSC656240 or Ar₂O₃ and Ad.mda-7 causes cell death irrespective of K-ras status, and cell death can be prevented by NAC administration.

FIG. 38. Combined treatment by NSC656240 or Ar₂O₃ and Ad.mda-7 does not affect the growth of normal cells.

FIG. 39. Combined treatment of pancreatic carcinoma cells by NSC656240 and Ad.mda-7 increases levels of annexin V in pancreatic cancer cells irrespective of K-ras status.

FIG. 40. NAC administration prevents the apoptosis of pancreatic cancer cells induced by treatment of pancreatic carcinoma cells by NSC656240 or Ar₂O₃ and Ad.mda-7.

FIG. 41. Combined treatment of pancreatic carcinoma cells by NSC656240 or Ar₂O₃ and Ad.mda-7 causes apoptosis irrespective of K-ras status.

FIG. 42. NSC656240 treatment, either alone or in combination with Ad.mda-7, does not down-regulate K-ras protein expression.

FIG. 43. MDA-7 expression in pancreatic cancer cell lines in the presence or absence of Ad.mda-7, Ad.K-rasAS, or NSC656243. The presence of MDA-7 in mutant K-ras cell lines requires expression of both mda-7 and the antisense K-ras construct, and is potentiated by NSC656243. The presence of MDA-7 in wild type K-ras cell lines does not require is not affected by K-ras AS expression, but is also potentiated by NSC656243 administration.

FIG. 44. MDA-7 expression in the pancreatic cancer cell lines PANC-1 and BxPC-3 in the presence or absence of Ad.mda-7, Ad.K-rasAS, or NSC656243.

FIG. 45. The MDA-7 expression observed in the PANC-1 or BxPC-3 cell lines following administration of Ad.mda-7 in combination with either NSC656240 or Ar₂O₃ is abrogated by NAC administration.

FIG. 46. Combined treatment by NSC656240 or Ar₂O₃ and Ad.mda-7 causes overproduction of ROS in pancreatic carcinoma cells.

FIG. 47. Combined treatment by NSC656240 or Ar₂O₃ and Ad.mda-7 causes overproduction of ROS in pancreatic carcinoma cells but not in normal cells.

FIG. 48. Combined treatment by NSC656240 or Ar₂O₃ and Ad.mda-7 causes overproduction of ROS in pancreatic carcinoma cells but not in immortalized astrocytes.

FIG. 49. Induction of apoptosis in pancreatic cancer cell lines (PANC-1) stably expressing MDA-7.

FIG. 50. Induction of apoptosis in pancreatic cancer cell lines (Mia PaCa-2) stably expressing MDA-7.

FIG. 51. Induction of apoptosis in pancreatic cancer cell lines (FM516 or BxPC-3) stably expressing MDA-7.

### 5. DETAILED DESCRIPTION OF THE INVENTION

The present invention relates to methods of treating cancer in a subject comprising generating, within one or more cancer cells of a subject, an effective amount of MDA-7 protein or functional equivalents thereof, and an effective amount of one or more free radicals. Non-limiting examples of means of generating effective amounts of MDA-7 within the target cell include the administration of mda-7 nucleic acid, MDA-7 protein, functional equivalents of these molecules, upregulation of an endogenous mda-7 gene, or stabilization of the mda-7 mRNA. Non-limiting examples of sources of free radicals include ionizing radiation, generators of free radicals, reactive oxygen species (ROS), generators of ROS, and disruptors of mitochondrial membrane potential. An effective amount of mda-7 nucleic acid or MDA-7 protein, as defined herein, is that amount which, together with an effective amount of one or more sources of free radicals, including ionizing radiation, free radicals, free radical generators, ROS, generators of ROS, or disruptors of mitochondrial membrane potential, inhibits cell proliferation and/or promotes cell death, preferably by at least about 10, 20, 30, 40, 50, 60, 70, 80 or 90 percent. The amount of inhibition of cell proliferation and/or promotion of cell death resulting from combined exposure to a mda-7 nucleic acid and/or a MDA-7 protein together with ionizing radiation, free radicals, generators of free radicals, ROS, generators of ROS, or disruptors of mitochondrial membrane potential is greater than the amount of inhibition of cell proliferation and/or promotion of cell death caused by mda-7 nucleic acid, MDA-7 protein, ionizing radiation, free radicals, generators of free radicals, ROS, generators of ROS, or disruptors of mitochondrial membrane potential when used alone, and in preferred, non-limiting embodiments, the magnitude of the combined effective agents are greater than additive relative to the effects of the uncombined agents. The term “effective” should not be construed to mean that the given amount of mda-7 nucleic acid or MDA-7 protein or source of free radicals, including ionizing radiation, a free radical, a free radical generator, a ROS, a ROS generator, and/or disruptor of mitochondrial membrane potential, when used alone, would be effective, as this might not be the case.

For clarity of description, and not by way of limitation, the detailed description of the invention is divided into the following subsections:

(1) mda-7 nucleic acids;

(2) MDA-7 proteins;

(3) free radical sources; and

(4) methods of treatment.

5.1 mda-7 Nucleic Acids

An mda-7 gene, as defined herein, is: 1) a nucleic acid as set forth in SEQ ID NO:1 (GenBank Accession No. U16261; Jiang et al., 1995, Oncogene 11:2477-2486); 2) a nucleic acid that encodes MDA-7, which in specific, non-limiting embodiments is a protein having 206 amino acids with a size of 23.8 kDa and an amino acid sequence as set forth in SEQ ID NO:2 (GenBank Accession No. U16261; Jiang et al., 1995, Oncogene 11:2477-2486); or 3) functional equivalents thereof. The mda-7 gene may be a genomic sequence containing introns but is more preferably a cDNA. The term mda-7 gene, as used herein, further encompasses nucleic acids preferably having between 400 and 2500 nucleotides, more preferably having at least 550, 600 or 650 nucleotides, which retain mda-7 function as a growth suppressant and pro-apoptotic molecule and which hybridize to a nucleic acid having a sequence as set forth in SEQ ID NO:1 under stringent hybridization conditions as set forth in “Current Protocols in Molecular Biology,” Volume 1, Ausubel et al., eds. John Wiley:New York N.Y. pp. 2.10.1-2.10.16, first published in 1989 but with annual updating, wherein maximum hybridization specificity for DNA samples immobilized on nitrocellulose filters may be achieved through the use of repeated washings in a solution comprising 0.1-2×SSC (15-30 mM NaCl, 1.5-3 mM sodium citrate, pH 7.0) and 0.1% SDS (sodium dodecylsulfate) at temperatures of 65-68° C. or greater. For DNA samples immobilized on nylon filters, a stringent hybridization washing solution may be comprised on 40 mM NaPO₄, pH 7.2, 1-2% SDS and 1 mM EDTA.

In preferred embodiments, mda-7 genes that hybridize under conditions of high stringency to the coding region of the nucleic acid sequence of SEQ ID NO:1 have at least about 70% sequence identity to the coding region of the nucleic acid sequence of SEQ ID NO:1, preferably at least 75%, more preferably at least 90%, and most preferably at least 95% sequence identitity to the coding region of the nucleic acid sequence of SEQ ID NO:1. The identity between two sequences is a direct function of the number of matching or identical positions. When a subunit position in both of the two sequences is occupied by the same monomeric subunit, e.g. if a given position is occupied by an adenine in each of two DNA molecules, then they are identical at that position. For example, if 7 positions in a sequence 10 nucleotides in length are identical to the corresponding positions in a second 10-nucleotide sequence, then the two sequences have 70% sequence identity. The length of comparison sequences will generally be at least 50 nucleotides, preferably at least 60 nucleotides, more preferably at least 75 nucleotides, and most preferably 100 nucleotides. Sequence identity is typically measured using sequence analysis software (e.g., Sequence Analysis Software Package of the Genetics Computer Group, University of Wisconsin Biotechnology Center, 1710 University Avenue, Madison, Wis. 53705) or other computer programs and/or algorithms known to those of ordinary skill in the art.

The term mda-7 gene as used herein further applies to nucleic acids containing terminal or internal deletions, insertions or substitutions, provided that those deletions, insertions or substitutions do not abrogate the ability of the protein encoded by the mda-7 gene to suppress the growth of or induce apoptosis or cell death in a given target cancer cell at a level relative to wild-type MDA-7 protein of at least about 10, 20, 30, 40, 50, 60, 70, 80 or 90 percent. For example, nucleic acids encoding a secreted form of MDA-7 lacking the N-terminal 48 amino acids of the coding sequence contained in SEQ ID NO:1 are known in the art (“secreted MDA-7” or “sMDA-7”) and are also an object of the instant invention, insofar as they retain at least about 10% of wild-type MDA-7 biological activity. Nucleic acids encoding proteins lacking approximately 5, 10, 15, 20 or 25% of the N- or C-terminal amino acids of MDA-7 are also objects of the instant invention, provided that they retain at least about 10% of wild-type MDA-7 biological activity.

As used herein, “MDA-7 biological activity” is defined as the ability to suppress growth and/or induce apoptosis and/or sensitize cells to the growth-suppressive or pro-apoptotic effects of radiation in a diverse group of transformed cell types without affecting these same properties in non-transformed cell types of similar origin. Examples of MDA-7 biological activity may be found, inter alia, in Su et al., 1998, Proc. Natl. Acad. Sci. U.S.A. 95:14400-14405 (breast cancer but not normal breast tissue) or Lebedeva et al., 2002, Oncogene 21:708-718 (melanoma but not melanocytes), the contents of which are incorporated by reference herein in their entireties.

In certain preferred embodiments, the mda-7 gene may be desirably comprised within a larger molecule. Thus, to render the mda-7 gene expressible, the gene may be linked to one of more elements that promote expression. For example, the gene may be operably linked to a suitable promoter element, such as, but not limited to, the cytomegalovirus immediate early (CMV) promoter, the Rous sarcoma virus (RSV) long terminal repeat promoter, the human elongation factor 1α promoter, the human ubiquitin c promoter, etc. It may be desirable, in certain embodiments of the invention, to use an inducible promoter. Non-limiting examples of inducible promoters include the murine mammary tumor virus promoter (inducible with dexamethasone), commercially-available tetracycline-responsive or ecdysone-responsive promoters, etc. It may also be desirable to utilize a promoter which is selectively active in the cancer cell to be treated, for example the PEG-3 gene promoter (U.S. Pat. No. 6,472,520). Examples of tissue- and cancer cell-specific promoters are well known to those of ordinary skill in the art.

Other elements that may be included in an mda-7-bearing vector include transcription start sites, stop sites, polyadenylation sites, ribosomal binding sites, etc.

Suitable expression vectors include virus-based vectors and non-virus based DNA or RNA delivery systems. Examples of appropriate virus-based vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2):113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention include, but are not limited to, so-called naked nucleic acids (Wolff et al., 1990, Science 247:1465-1468), nucleic acids encapsulated in liposomes (Nicolau et al., 1987, Methods in Enzymology 149:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

In specific, non-limiting embodiments of the invention, the expression vector is an E1-deleted human adenovirus vector of serotype 5, although those of ordinary skill in the art would recognize that many of the different naturally-occurring human Ad serotypes or Ad vectors derived from non-human adenoviruses may substitute for human Ad 5-derived vectors. In a preferred, specific, non-limiting embodiment, a recombinant replication-defective Ad.mda-7 virus for use as an mda-7 vector may be created in two steps as described in Su et al., 1998, Proc. Natl. Acad. Sci. U.S.A. 95:14400-14405. Specifically, the coding region of the mda-7 gene may be cloned into a modified Ad expression vector pAd.CMV (Falck-Pedersen et al., 1994, Mol. Pharmacol. 45:684-689). This vector contains, in order, the first 355 bp from the left end of the Ad genome, the CMV promoter, DNA encoding splice donor and acceptor sites, the coding region of the mda-7 cDNA, DNA encoding a polyA signal sequence from the β globin gene, and ˜3 kbp of adenovirus sequence extending from within the E1B coding region. This arrangement allows high-level expression of the cloned sequence by the CMV promoter, and appropriate RNA processing. The recombinant virus may be created in vitro in 293 cells (Graham et al., 1977, J. Gen. Virol. 36:59-72) by homologous recombination between an mda-7-containing version of pAd.CMV and plasmid pJM17, which contains the whole of the Ad genome cloned into a modified version of pBR322 (McGrory et al., 1988, Virology 163:614-617). pJM17 gives rise to Ad genomes in vivo, but they are too large to be packaged in mature Ad capsids. This constraint is relieved by recombination with the vector to create a packageable genome (Id.) containing the mda-7 gene. The recombinant virus is replication defective in human cells except 293 cells, which express adenovirus E1A and E1B. Following transfection of the two plasmids, infectious virus may be recovered, and the genomes may be analyzed to confirm the recombinant structure, and then virus may be plaque purified by standard procedures (Volkert and Young, 1983, Virology 125:175-193).

In a specific, non-limiting embodiment of the invention, the infectivity of an adenovirus vector carrying an mda-7 gene may be improved by inserting an Arg-Gly-Asp motif into the fiber know (Ad5-Delta24RGD), as described in Lamfers et al., 2002, Cancer Res. 62:5736-5742.

A nucleic acid comprising an mda-7 gene, as described above, may be introduced into at least one cancer cell of a subject by methods known in the art. For example, but not by way of limitation, a solution comprising an effective amount of the nucleic acid comprising mda-7 may be introduced (i) into a cavity resulting from the complete or partial surgical excision of a tumor mass, (ii) into a tumor mass by direct intratumoral injection, (iii) into the bloodstream of the subject, or (iv) into the extracellular space, if any, surrounding the tumor. In preferred specific embodiments of the invention, infection of the target cell may be achieved by exposure to approximately 100 plaque-forming units of an adenovirus vector comprising an mda-7 gene.

5.2 MDA-7 Proteins

The term “MDA-7” as used herein refers to a protein encoded by a mda-7 nucleic acid as defined hereinabove. In one specific, non-limiting embodiment, MDA-7 has essentially the amino acid sequence of SEQ ID NO:2 as provided in Genbank Accession Number U16261 (“wtMDA-7”), or a functional equivalent thereof. A “functional equivalent” of the MDA-7 protein is a polypeptide whose sequence is altered by any deletion, insertion, and/or addition that does not destroy the MDA-7 biological activity of the polypeptide. “MDA-7 biological activity,” as defined hereinabove, is the ability to suppress growth and/or induce apoptosis and/or sensitize cells to the growth-suppressive or pro-apoptotic effects of radiation in a diverse group of transformed cell types without affecting these same properties in non-transformed cell types of similar origin. One type of functional equivalent of MDA-7 contains terminal or internal deletions, insertions or substitutions of amino acids, preferably involving up to about 1, 5, 10, 20, 25, or 30% of the total number of amino acids of the wtMDA-7 protein, provided that these deletions, insertions or substitutions do not abrogate the ability of the protein encoded by the mda-7 gene to suppress the growth of or induce apoptosis or cell death in a given target cancer cell at a level relative to wild-type MDA-7 protein of at least about 10, 20, 30, 40, 50, 60, 70, 80 or 90 percent. A specific non-limiting example of such a functional equivalent is secreted MDA-7 (“sMDA-7”), which lacks the 48 amino acids comprising the N-terminus of the MDA-7 polypeptide.

Another type of functional equivalent is MDA-7 comprised in a fusion protein. A specific, non-limiting example of a functional equivalent of wt MDA-7 is GST-MDA-7, produced by an expression system wherein MDA-7 is fused to glutathione-S-transferase. More preferably, the secretory sequence of MDA-7 is deleted in the GST-MDA-7 fusion protein.

MDA-7 protein for use according to the invention may be produced using any method known in the art. A nucleic acid encoding MDA-7, operably linked to a suitable promoter element, may be comprised in an expression vector, and the expression vector may then be introduced into a suitable host cell for expression of MDA-7. MDA-7 protein may be produced in vitro or, alternatively, the host cell may be comprised in the subject to be treated such that MDA-7 protein is produced in vivo.

Suitable expression vectors for producing MDA-7 protein include virus-based vectors and non-virus based DNA or RNA delivery systems. Examples of appropriate virus-based vectors include, but are not limited to, those derived from retroviruses, for example Moloney murine leukemia-virus based vectors such as LX, LNSX, LNCX or LXSN (Miller and Rosman, 1989, Biotechniques 7:980-989); lentiviruses, for example human immunodeficiency virus (“HIV”), feline leukemia virus (“FIV”) or equine infectious anemia virus (“EIAV”)-based vectors (Case et al., 1999, Proc. Natl. Acad. Sci. U.S.A. 96: 22988-2993; Curran et al., 2000, Molecular Ther. 1:31-38; Olsen, 1998, Gene Ther. 5:1481-1487; U.S. Pat. Nos. 6,255,071 and 6,025,192); adenoviruses (Zhang, 1999, Cancer Gene Ther. 6(2): 113-138; Connelly, 1999, Curr. Opin. Mol. Ther. 1(5):565-572; Stratford-Perricaudet, 1990, Human Gene Ther. 1:241-256; Rosenfeld, 1991, Science 252:431-434; Wang et al., 1991, Adv. Exp. Med. Biol. 309:61-66; Jaffe et al., 1992, Nat. Gen. 1:372-378; Quantin et al., 1992, Proc. Natl. Acad. Sci. U.S.A. 89:2581-2584; Rosenfeld et al., 1992, Cell 68:143-155; Mastrangeli et al., 1993, J. Clin. Invest. 91:225-234; Ragot et al., 1993, Nature 361:647-650; Hayaski et al., 1994, J. Biol. Chem. 269:23872-23875; Bett et al., 1994, Proc. Natl. Acad. Sci. U.S.A. 91:8802-8806), for example Ad5/CMV-based E1-deleted vectors (Li et al., 1993, Human Gene Ther. 4:403-409); adeno-associated viruses, for example pSub201-based AAV2-derived vectors (Walsh et al., 1992, Proc. Natl. Acad. Sci. U.S.A. 89:7257-7261); herpes simplex viruses, for example vectors based on HSV-1 (Geller and Freese, 1990, Proc. Natl. Acad. Sci. U.S.A. 87:1149-1153); baculoviruses, for example AcMNPV-based vectors (Boyce and Bucher, 1996, Proc. Natl. Acad. Sci. U.S.A. 93:2348-2352); SV40, for example SVluc (Strayer and Milano, 1996, Gene Ther. 3:581-587); Epstein-Barr viruses, for example EBV-based replicon vectors (Hambor et al., 1988, Proc. Natl. Acad. Sci. U.S.A. 85:4010-4014); alphaviruses, for example Semliki Forest virus- or Sindbis virus-based vectors (Polo et al., 1999, Proc. Natl. Acad. Sci. U.S.A. 96:4598-4603); vaccinia viruses, for example modified vaccinia virus (MVA)-based vectors (Sutter and Moss, 1992, Proc. Natl. Acad. Sci. U.S.A. 89:10847-10851) or any other class of viruses that can efficiently transduce human tumor cells and that can accommodate the nucleic acid sequences required for therapeutic efficacy.

Non-limiting examples of non-virus-based delivery systems which may be used according to the invention to produce MDA-7 protein include, but are not limited to, so-called naked nucleic acids (Wolff et al., 1990, Science 247:1465-1468), nucleic acids encapsulated in liposomes (Nicolau et al., 1987, Methods in Enzymology 149:157-176), nucleic acid/lipid complexes (Legendre and Szoka, 1992, Pharmaceutical Research 9:1235-1242), and nucleic acid/protein complexes (Wu and Wu, 1991, Biother. 3:87-95).

MDA-7 protein also may be produced by yeast or bacterial expression systems. For example, bacterial expression may be achieved using plasmids such as pCEP4 (Invitrogen, San Diego, Calif.), pMAMneo (Clontech, Palo Alto, Calif.; see below), pcDNA3.1 (Invitrogen, San Diego, Calif.), etc.

Depending on the expression system used, nucleic acid may be introduced by any standard technique, including transfection, transduction, electroporation, bioballistics, microinjection, etc.

MDA-7 protein may be used in the context of a culture supernatant, or a partially or essentially completely purified protein. Standard techniques may be used to purify the protein.

In specific, non-limiting embodiments of the invention, the expression vector is an E1-deleted human adenovirus vector of serotype 5, although those of ordinary skill in the art would recognize that many of the different naturally-occurring human Ad serotypes or Ad vectors derived from non-human adenoviruses may substitute for human Ad 5-derived vectors. To prepare a human Ad 5-derived vector, an expression cassette comprising a transcriptional promoter element operatively linked to an MDA-7 coding region and a polyadenylation signal sequence may be inserted into the multiple cloning region of an adenovirus vector shuttle plasmid, for example pXCJL.1 (Berkner, 1988, Biotechniques 6:616-624). In the context of this plasmid, the expression cassette may be inserted into the DNA sequence homologous to the 5′ end of the genome of the human serotype 5 adenovirus, disrupting the adenovirus E1 gene region. Transfection of this shuttle plasmid into the E1-transcomplementing 293 cell line (Graham et al., 1977, J. General Virology 36:59-74), or another suitable cell line known in the art, in combination with either an adenovirus vector helper plasmid such as pJM17 (Berkner, 1988, Biotechniques 6:616-624; McGrory et al., 1988, Virology 163:614-617) or pBHG10 (Bett et al., 1994, Proc. Natl. Acad. Sci. U.S.A. 91: 8802-8806) or a ClaI-digested fragment isolated from the adenovirus 5 genome (Berkner, 1988, Biotechniques 6:616-624), allows recombination to occur between homologous adenovirus sequences contained in the adenovirus shuttle plasmid and either the helper plasmid or the adenovirus genomic fragment. This recombination event gives rise to a recombinant adenovirus genome in which the cassette for the expression of the foreign gene has been inserted in place of a functional E1 gene. When transcomplemented by the protein products of the human adenovirus type 5 E1 gene (for example, as expressed in 293 cells), these recombinant adenovirus vector genomes can replicate and be packaged into fully-infectious adenovirus particles. The recombinant vector can then be isolated from contaminating virus particles by one or more rounds of plaque purification (Berkner, 1988, Biotechniques 6:616-624), and the vector can be further purified and concentrated by density ultracentrifugation.

In a specific, non-limiting embodiment of the invention, an mda-7 nucleic acid, in expressible form, may be inserted into the modified Ad expression vector pAd.CMV (Falck-Pedersen et al., 1994, Mol. Pharmacol. 45:684-689). This vector contains, in order, the first 355 base pairs from the left end of the adenovirus genome, the cytomegalovirus immediate early promoter, DNA encoding splice donor and acceptor sites, a cloning site for the mda-7 gene, DNA encoding a polyadenylation signal sequence from the beta globin gene, and approximately three kilobase pairs of adenovirus sequence extending from within the E1B coding region. This construct may then be introduced into 293 cells (Graham et al., 1977, J. Gen. Virol. 36:59-72) together with plasmid pJM17 (above), such that, as explained above, homologous recombination can generate a replication defective adenovirus containing MDA-7-encoding nucleic acid.

In another specific, non-limiting embodiment of the invention, where mda-7 is to be introduced into cells in culture, a suitable expression vector may be prepared by inserting an mda-7 nucleic acid, extending from nucleotide 176 to nucleotide 960 in the sequence presented as SEQ ID NO:1, encoding the open reading frame, into pCEP4 (Invitrogen, San Diego, Calif.) downstream of the CMV promoter. Another suitable vector may be the Rous sarcoma virus (“RSV”) vector available as pREP4 (Invitrogen).

In specific preferred non-limiting embodiments, standard cloning procedures may be used to generate a bacterial expression vector comprising in-frame fusion of the mda-7 ORF 3′ to the GST ORF in GST-4T2 vector (Amersham Pharmacia), using BamHI and NotI sites introduced into mda-7 by PCR. Expression of protein may be performed by inoculating an overnight culture at 1:100 dilution followed by incubation at 25° C. until an O.D. 600 of 0.4-0.6 was reached followed by induction with 0.1 μM IPTG for 2 h. Cells may be harvested by centrifugation and sonicated in PBS followed by centrifugation to obtain soluble protein. The lysate may be bound to a glutathione-agarose column (Amersham Pharmacia) at 4° C. overnight followed by washing with 50 volumes PBS and 10 volumes PBS with 500 mM NaCl. Elution of bound protein may be performed by passing 20 mM-reduced Glutathione through the column and collecting 1 ml fractions. Fractions may be analyzed by gel electrophoresis and positive samples may be dialyzed against 1000 volumes of PBS for 4 h with one change followed by 500 volumes of DMEM for 4 h. MDA-7 may be freed of GST using a site-specific protease that recognizes a region upstream of the multiple-cloning site of pGEX plasmids. Detailed information regarding the use of the pGEX system may be found in the GST Gene Fusion Handbook published by Amersham Biosciences.

If produced in vivo, MDA-7 may be present in the extracellular fluid of a cancer cell to be treated.

5.3 Free Radical Sources

Concurrent with, prior to, or after exposure of the cancer cell to an effective amount of a mda-7 nucleic acid, MDA-7 protein, or compounds that induce the expression of the mda-7 gene, the cancer cell also may be exposed, in vivo or ex vivo, to an effective amount of one or more sources of free radicals, including but not limited to ionizing radiation, a free radical, a generator of a free radical, a ROS, a generator of a ROS, or a disruptor of mitochondrial membrane potential.

An effective amount of one or more sources of free radicals is that amount which, together with an effective amount of mda-7 nucleic acid or MDA-7 protein, inhibits cell proliferation and/or promotes cell death, preferably by at least about 10, 20, 30, 40, 50, 60, 70, 80 or 90 percent. The amount of inhibition of cell proliferation and/or promotion of cell death resulting from combined exposure to the one or more sources of free radicals and the mda-7 nucleic acid and/or a MDA-7 protein is greater than the amount of inhibition of cell proliferation and/or promotion of cell death caused by the exposure to the one or more sources of free radicals, mda-7 nucleic acid, MDA-7 protein, ionizing radiation, free radicals, generators of free radicals, ROS, generators of ROS, or disruptors of mitochondrial membrane potential when used alone, and in preferred, non-limiting embodiments, the magnitude of the combined effective agents are greater than additive relative to the effects of the uncombined agents. The term “effective” should not be construed to mean that the given amount of one or more sources of free radicals, mda-7 nucleic acid, or MDA-7 protein, when used alone, would be effective, as this might not be the case.

Sources of free radicals that effectively synergize with mda-7 nucleic acid or MDA-7 protein may be identified by one of ordinary skill in the art by administering to a cultured cell line known to be susceptible to mda-7 nucleic acid or MDA-7 protein in combination with one or more sources of free radicals, for example but not by way of limitation the human glioma cell lines U87MG, U251MG, U373MG or T98G, a test source of free radicals in combination with an effective amount of mda-7 nucleic acid or an effective amount of the MDA-7 protein. Administration of the test source of free radicals is performed in the presence or absence of a known inhibitor of free radicals, for example but not by way of limitation, N-acetyl-cysteine. A source of free radicals encompassed by the present invention would be one whose anti-proliferative or pro-apoptotic activity in combination with mda-7 nucleic acid or MDA-7 protein is reduced or prevented by administration of N-acetyl-cysteine or any other known inhibitor of free radicals.

Examples of free radical generators include, but are not limited to arsenic trioxide, NSC656240, 4-HPR, and cisplatin. Examples of ROS include but are not limited to singlet oxygen, hydrogen peroxide, superoxide anion, hydroxyl radicals, peroxynitrite, and oxidants.

In preferred embodiments, the free radical generators are arsenic trioxide, NSC656240 or 4-HPR. In other preferred embodiments, the disruptor of mitochondrial membrane potential is PK 11195.

5.4 Methods of Treatment

Cancers that may be treated according to the present invention include but are not limited to all MDA-7-responsive cancers that are sensitive to one or more sources of free radicals, including ionizing radiation, free radicals, generators of free radicals, ROS, generators of ROS, and/or disruptors of mitochondrial membrane potential. In addition, the invention may be applied to other cancers wherein any of these agents alone may be ineffective, but the combination of MDA-7 and one or more of these other agents causes cancer cell death. Examples of cancers that are fully, partially or conditionally responsive to MDA-7 include, but are not limited to, melanoma, breast cancer, pancreatic cancer, prostate cancer, glioblastoma, lung cancer (including but not limited to small cell and non-small cell (adenocarcinoma, squamous cell, and large cell) varieties), Hodgkin's lymphoma, non-Hodgkins lymphoma, cancer of the esophagus, head and neck cancer, thyroid cancer, leukemia, ovarian cancer, testicular cancer, gastric cancer, liver cancer (including but not limited to hepatocellular carcinoma, cholangiocarcinoma, and angiosarcoma), sarcomas, renal cancer, bladder cancer, and colorectal cancer. Where the cancer cell target carries an activating mutation in ras, in specific, non-limiting embodiments, an inhibitor of ras may be administered to the cell, for example as set forth in International Patent Application No. PCT/US02/26454, Publication No. WO 0316499, incorporated by reference herein.

Examples of cancers that are, or may be, sensitive to one or more sources of free radicals, including, but not limited to, ionizing radiation, free radicals, generators of free radicals, ROS, generators of ROS, or disruptors of mitochondrial membrane potential include, but are not limited to, acute promyelocytic leukemia, bladder cancer, cervical cancer, colorectal cancer, esophageal cancer, gastric cancer, glioma, hepatocellular carcinoma, lung adenocarcinoma, multiple myeloma, nasopharyngeal cancer, neuroblastoma, osteosarcoma, ovarian cancer, progranulocytic leukemia, prostate cancer, renal cell carcinoma, retinoblastoma, rhabdomyosarcoma, squamous cell carcinoma, and transitional cell carcinoma. In preferred non-limiting embodiments, the invention is used in the treatment of renal cell carcinoma, glioblastoma multiforme, ovarian cancer, prostate cancer, pancreatic cancer, and colorectal cancer.

The one or more sources of free radicals may be administered intravenously, intratumorally, intraperitoneally, parenterally, intramuscularly, subcutaneously, or by any other suitable route of administration. When the free radical generator is ionizing radiation, an effective dose of radiation may be provided as used in the standard radiotherapy for the tumor or cancer to be treated, either externally or intraoperatively.

The interval between introduction of mda-7 nucleic acid or MDA-7 protein and exposure to radiation may be at least about 30 minutes, or at least about 6-12 hours, and more preferably at least about 24 hours to 7 days. In non-limiting embodiments, a cancer cell may be exposed to between 2 and 100 Gy in a single treatment or as a result of multiple treatments. In one specific non-limiting embodiment of the invention, one external treatment of 2.0 Gy may be administered each of 5 days a week for six weeks for a total of 60 Gy. If intraoperative radiation is administered, the amount administered may be between 3 and 15 Gy total, and preferably 6 Gy.

When the free radical generator is arsenic trioxide, it should be administered intravenously, intratumorally or intraperitoneally at doses between about 0.05 to 0.5 mg/kg/day, or more preferably between about 0.10 and 0.25 mg/kg/day, and most preferably of about 0.15 mg/kg/day or less, with the dose being adjusted as necessary to achieve an extracellular concentration of between about 0.01 and 10 μM, or preferably between about 0.1 and 1.0 μM or less at the target cell. For other agents, an effective amount, in the presence of MDA-7, preferably but not by limitation decreases the viability of a cancer cell line by at least about 20% or 30% relative to MDA-7 alone.

According to the invention, the amount of MDA-7 protein in a cell may be increased by exposing the cancer cell to an effective amount of a mda-7 nucleic acid, in expressible form, or MDA-7 protein. An effective amount is an amount which, when administered with one or more sources of free radicals ultimately has a growth suppressive effect on the cancer cell. An effective amount of a mda-7 nucleic acid or MDA-7 protein may exert its own growth suppressive effect, but this is not necessary to practice within the scope of this invention. Rather, the amount of a mda-7 nucleic acid or MDA-7 protein, to be effective, merely must enhance the effectiveness of the one or more sources of free radicals in suppressing cell proliferation and/or promoting apoptosis. Effective amounts may be determined using techniques known to those of skill in the art, and may include, for example but not by way of limitation, in vivo animal models or in vitro assays using cultured cell lines.

For example, and not by way of limitation, where mda-7 is to be introduced into a cancer cell via a viral vector, the amount of virus to which the cell is exposed may be between about 1-100,000 viral particles or plaque-forming units (pfu) per cell, and preferably between about 100-250 pfu/cell. In the working examples set forth in Section 6, below, 100 pfu/cell of mda-7 comprised in a replication-defective adenovirus vector was used.

Where MDA-7 is to be introduced into the cancer cell as a protein, the dose of the protein may be between 0.05 to 50, or between 0.1 and 10, or preferably between about 0.1 and 1, micrograms of protein per milliliter, or concentrations of MDA-7 or MDA-7/GST fusion protein of between about 0.05 to 5 nM, or between about 0.1 and 1 nM, or preferably of about 0.5 nM. The protein may be comprised in a suitable carrier. Alternatively, MDA-7 may be comprised in extracellular fluid of a cancer cell to be treated.

To expose a cancer cell to an effective amount of mda-7 nucleic acid or MDA-7 protein, the mda-7 nucleic acid or MDA-7 protein may be introduced into a subject either by direct injection into a tumor or in proximity to cancer cells; by infusion into a site of partial or complete tumor excision; by infusion into a subject's bloodstream; by infusion into a body space, such as into the peritoneum, gastric, pulmonary or intestinal lavage, instillation into the bladder, injection into bone marrow, or infusion into cerebrospinal fluid; or by introducing, in vivo or ex vivo, an mda-7 gene, in expressible form, into a host cell, which may be non-malignant or rendered non-dangerous by irradiation, encapsulation, etc. The host cell may normally reside in proximity to cancer cells to be treated or which may be placed in such position. The host cell, by producing and secreting MDA-7, provides MDA-7 protein to cells to be treated. Sufficient host cells expressing mda-7 gene at sufficient levels may be used to provide an effective amount of MDA-7 protein.

Alternatively, a cancer cell may be exposed to mda-7 nucleic acid or MDA-7 protein ex vivo. For example, a cancer cell comprised in a population of cells heterogeneous for malignant and non-malignant cells may be collected from a subject, treated ex vivo according to the invention, and then reintroduced into the subject, with an aim toward selectively destroying malignant cells.

Treatment with mda-7 nucleic acid or MDA-7 protein and one or more sources of free radicals, including but not limited to ionizing radiation, a free radical, a free radical generator, a ROS, a generator of a ROS, or a disrupter of mitochondrial membrane potential may be combined with other forms of therapy, including but not limited to surgery, gene therapy, chemotherapy, and anti-sense therapy.

The mda-7 nucleic acid or MDA-7 protein may be provided as a pharmaceutical composition, comprising the mda-7 gene in expressible form, the MDA-7 protein, or a combination thereof, together with pharmaceutically acceptable carriers or excipients, in a pharmaceutically acceptable sterile vehicle. Pharmaceutically acceptable carriers include aqueous solutions, non-toxic excipients, including salts, preservatives, buffers and the like, as described in Remington's Pharmaceutical Sciences, pp. 1405-12 and 1461-87 (1975) and The National Formulary XIV, 14th Ed. Washington: American Pharmaceutical Association (1975). Aqueous carriers include water, alcoholic/aqueous solutions, saline solutions, parenteral vehicles such as sodium chloride, Ringer's dextrose, etc. The pharmaceutical composition may be preferably administered to patients via continuous intravenous infusion, but can also be administered by single or multiple injections. Intravenous vehicles include fluid and nutrient replenishers. Preservatives include antimicrobials, anti-oxidants, chelating agents and inert gases. The pH and exact concentration of the various components of the composition are adjusted according to routine skills in the art. See Goodman and Gilman's The Pharmacological Basis for Therapeutics (7th ed.). The nulceic acids or peptides of the present invention may be present in pharmaceutical compositions in a concentration of approximately 0.1 to 99.9% by weight, specifically 0.5 to 95% by weight, relative to the total mixture. Such pharmaceutical compositions also may comprise other pharmaceutically active substances in addition to the nucleic acids or peptides of the present invention. Other methods of delivering the pharmaceutical compositions to patients also will be readily apparent to the skilled artisan.

### 6. WORKING EXAMPLES

### 6.1. Example 1

### Radiotherapy in Combination with MDA-7 Nucleic Acid or Protein as a Treatment for Glioblastoma Multiforme

Materials and Methods

Cell Lines and Culture Conditions. Fetal astrocytes were isolated from second trimester (gestational age 16-19 wk) human fetal brains obtained from elective abortions in full compliance with NIH guidelines, as previously described (Bencheikh et al., 1999, NeuroVirol. 5:115-124; Canki et al., 2001, J. Virol. 75:7925-7933; Su et al., 2002, Oncogene 21:3592-3602). Highly homogenous preparations of primary normal human fetal astrocytes, PHFA, were obtained using high-density culture conditions in the absence of growth factors in F12 Dulbecco's Modified Eagle Medium (GIBCO-BRL, Gaithersburg, Md.) containing 10% fetal bovine serum (FBS), penicillin, streptomycin, and gentamycin. Cells were maintained in this medium at 2-5×10⁴ cells/cm² and subcultured weekly up to 6 times. Cultures were regularly monitored for expression of the astrocytic marker glial fibrillary acidic protein (GFAP) and either HAM56 or CD68 to identify cells of monocyte/macrophage lineage. Only cultures that contained ≧99% GFAP positive astrocytes and rare or no detectable HAM56 or CD68 positive cells were used in experiments (Canki et al., 2001, J. Virol. 75:7925-7933; Su et al., 2002, Oncogene 21:3592-3602). A putative immortalized clone of PHFA, PHFA-Im, was obtained by infection with a retrovirus (pBabe-hygro-hTERT) expressing the human telomerase (hTERT) gene (Hahn et al., 1999, Nature 400:464-468). Pseudotyped retrovirus was obtained by transfection of 293GPG cells with pBabe-hygro-hTERT plasmid as described (Ory et al., 1996, Proc. Natl. Acad. Sci. USA 93:11400-11406), which were used for infection of PHFA cells and selection of clones with hygromycin at 200 μg/ml for two weeks, followed by isolation of individual colonies using cloning cylinders. These were expanded and frozen at low passage. Cell lines were tested for expression of hTERT by Northern blot analyses and survival of colonies over several passages (primary astrocytes do not survive extended serial passage >˜10). PHFA-Im cells have now been cultured for >50 passages, whereas proliferation of PHFA usually did not occur past ˜7 passages. The wtp53 human glioma cell line U87MG and the mutp53 human glioma cell lines U251MG, U373MG and T98G were obtained from the American Type Culture Collection. These cell lines were grown in Dulbecco's modified Eagle's medium supplemented with 10% fetal bovine serum (DMEM-10) at 37° C. in a 95% air 5% CO₂ humidified incubator.

Virus Construction, Plaque Assays and Virus Infection Protocol. The recombinant replication-defective Ad.mda-7 virus was created in two steps as described previously (Su et al., 1998, Proc. Natl. Acad. Sci. U.S.A. 95:14400-14405). Briefly, the coding region of the mda-7 gene was cloned into a modified Ad expression vector pAd.CMV (Falck-Pedersen et al., 1994, Mol. Pharmacol. 45:684-689). This vector contains, in order, the first 355 bp from the left end of the Ad genome, the cytomegalovirus immediate early promoter, DNA encoding splice donor and acceptor sites, the coding region of the mda-7 cDNA, DNA encoding a polyA signal sequence from the β globin gene, and ˜3 kbp of adenovirus sequence extending from within the E1B coding region. This arrangement allows high-level expression of the cloned sequence by the cytomegalovirus immediate early gene promoter, and appropriate RNA processing (Falck-Pedersen et al., 1994, Mol. Pharmacol. 45:684-689). The recombinant virus was created in vivo in 293 cells (Graham et al., 1977, J. Gen. Virol. 36:59-72) by homologous recombination between mda-7-containing vector and plasmid pJM17, which contains the whole of the Ad genome cloned into a modified version of pBR322 (McGrory et al., 1988, Virology 163:614-617). pJM17 gives rise to Ad genomes in vivo but they are too large to package. This constraint is relieved by recombination with the vector to create a packageable genome (McGrory et al., 1988, Virology 163:614-617), containing the mda-7 gene. The recombinant virus is replication defective in human cells except 293 cells, which express adenovirus E1A and E1B. Following transfection of the two plasmids, infectious virus was recovered, the genomes were analyzed to confirm the recombinant structure, and then virus was plaque purified, all by standard procedures (Volkert and Young, 1983, Virology 125:175-193). Stock virus preparations were diluted in the appropriate growth medium in the presence or absence of 1% fetal bovine serum and inoculated onto cell monolayers at the indicated m.o.i. After 1-3 hr virus adsorption at 37° C. with rotation every 15 min, the virus inoculum was removed and DMEM-10 was added to the infected monolayer cultures and cells were incubated at 37° C. for the indicated times.

RNA Isolation and Northern Blotting Assays. For determining temporal effects of mda-7 mRNA expression following Ad.mda-7 infection, total cellular RNA was isolated by the guanidinium/phenol extraction method and Northern blotting was performed as described in Su et al., 1997, Proc. Natl. Acad. Sci. USA 94:9125-9130. Fifteen μg of RNA were denatured and electrophoresed in 1.2% agarose gels with 3% formaldehyde, transferred to nylon membranes and hybridized sequentially with ³²P labeled mda-7 cDNA probe, the blot was stripped and reprobed with a ³²P-labeled gapdh probe as described previously (Su et al., 1997, Proc. Natl. Acad. Sci. USA 94:9125-9130; Su et al., 1999, Proc. Natl. Acad. Sci. USA 96:15115-15120). For analysis of GADD family gene expression, total RNA was extracted from the cells by Qiagen RNeasy mini kit according to the manufacturer's protocol. Ten μg of total RNA for each sample was used for Northern blotting as previously described (Su et al., 1997, Proc. Natl. Acad. Sci. USA 94:9125-9130). Blots were probed with α-³²P[dCTP]-labeled cDNA probes, including, full-length human mda-7, GADD153, GADD45α and a 500-bp fragment from human GADD34. Blots were stripped and reprobed with an α-³²P[dCTP]-labeled human GAPDH probe. Following hybridization, the filters were washed and exposed for autoradiography.

Cell Cycle Analysis. Cells were trypsinized, washed 2× with PBS and fixed in 70% ethanol overnight at −20° C. Then cells were washed 2× with PBS, and aliquots of 1×10⁶ cells were resuspended in 1 ml of PBS containing 1 mg/ml of RNase A and 0.5 mg/ml of propidium iodide. After 30 min incubation, cells were analyzed by flow cytometry using a FACScan flow cytometer (Becton Dickinson, San Jose, Calif.).

Annexin-V Binding Assay. Cells were trypsinized and washed once with complete media. The aliquots of the cells (5×10⁵) were resuspended in complete media (0.5 ml) and stained with FITC-labeled Annexin-V (kit from Oncogene Research Products, Boston, Mass.) according to the manufacturer's instructions. Propidium iodide (PI) was added to the samples after staining with Annexin-V to exclude late apoptotic and necrotic cells. The FACS assay was performed immediately after staining.

Detection of CAR Receptors on the Cell Surface. Cells were harvested by trypsinization, washed 2× with PBS and stained with 1:1000 dilution of monoclonal anti-CAR antibody, for 1 h at 37° C. After additional washings with PBS (2×5 min), cells were stained in the dark for 1 h at 37° C. with a 1:500 dilution of fluorescein-labeled goat anti-mouse immunoglobulins (BioSource International, Camarillo, Calif.). After washings with PBS, cells were analyzed by flow cytometry (5×10⁵ cells per sample). Unstained cells and cells stained with monoclonal anti-SV40 antibody (Santa Cruz, Calif.) and with secondary antibody only were used as controls. Two methods were employed for semi-quantitation of the results (Lebedeva et al., 2002, Oncogene 21:708-718). The shift of FL1 peak was calculated as difference in peak channels between the control (cells unstained or stained with non-specific antibody) and the experimental cells stained with anti-CAR antibody relative to the position of the control cell peak channel. The second method uses the Kolmogorov-Smimov (K-S) test for overlaid histograms (Young, 1977, J. Histochem. Cytochem 25:935-941). The calculation computes the summation of the overlaid curves and determines the greatest difference between the summation curves (K-S statistics). D value indicates the greatest difference between the two curves.

TUNEL Assay. After the different treatment protocols, cells were trypsinized, washed twice with PBS/1% BSA and resuspended in PBS/1% BSA. Suspended cells (1×10⁶) were fixed with an equal amount of a freshly prepared paraformaldehyde solution (4% in PBS, pH 7.4). After 30 min incubation at room temperature, cells were washed with PBS and resuspended in permeabilization solution (0.1% Triton® X-100 in 0.1% sodium citrate) for 2 min on ice. After another wash with PBS, cells were labeled using fluorescein cell death detection kit (Boehringer Mannheim) according to the manufacturer's instructions. After the labeling, cells were analyzed by flow cytometry using a FACScan flow cytometer (Becton Dickinson, San Jose, Calif.).

Expression, Purification and Analysis of GST-MDA-7 Protein. Standard cloning procedures were used to generate a bacterial expression vector comprising in-frame fusion of the mda-7 ORF 3′ to the GST ORF in GST-4T2 vector (Amersham Pharmacia), using BamHI and NotI sites introduced into mda-7 by PCR. Expression of protein was performed by inoculating an overnight culture at 1:100 dilution followed by incubation at 25° C. until an O.D. 600 of 0.4-0.6 was reached followed by induction with 0.1 μM IPTG for 2 h. Cells were harvested by centrifugation and sonicated in PBS followed by centrifugation to obtain soluble protein. The lysate was bound to a Glutathione-agarose column (Amersham Pharmacia) at 4° C. overnight followed by washing with 50 volumes PBS and 10 volumes PBS with 500 mM NaCl. Elution of bound protein was performed by passing 20 mM-reduced Glutathione through the column and collecting 1 ml fractions. Fractions were analyzed by gel electrophoresis and positive samples were dialyzed against 1000 volumes of PBS for 4 h with one change followed by 500 volumes of DMEM for 4 h. Protein concentration was estimated by Bradford assays as well as gel electrophoresis in conjunction with Coomassie blue staining. Samples were tested for activity using GST protein as control. Each batch of protein had a certain effective working concentration (cell killing) ranging from 2 to 10 μg/ml.

Western Blot Analysis. Western blot assays were performed as previously described (Lebedeva et al., 2002, Oncogene 21:708-718). Cells were washed 2× with cold PBS and lysed on ice for 30 min in 100 μl of cold RIPA buffer [50 mM Tris-HCl (pH 8.0), 150 mM NaCl, 0.1% SDS, 1% NP40, and 0.5% sodium deoxycholate] with freshly added 0.1 mg/ml phenylmethylsulfonyl fluoride, 1 mM sodium orthovanadate, and 1 mg/ml aprotinin. Cell debris were removed by centrifugation at 14,000 g for 10 min at 4° C. Protein concentrations were determined using the Bio-Rad protein assay system (Bio-Rad Laboratories, Richmond, Calif.). Aliquots of cell extracts containing 20-50 mg of total protein were resolved in 12% SDS-PAGE and transferred to Immobilon-P PVDF membranes (Millipore Corp., Bedford, Mass.). Filters were blocked for 1 h at room temperature in Blotto A [5% nonfat milk powder in TBS-T: 10 mM Tris-HCl pH 8.0), 150 mM NaCl, 0.05% Tween 20), and then incubated for 1 h at room temperature in Blotto A containing a 1:1000 dilution of rabbit anti MDA-7, anti-Bcl-2, anti-Bcl-XL, anti-BAX, or anti-BAK polyclonal antibodies. After washing in TBS-T buffer (3×5 min, RT), filters were incubated for 1 h at room temperature in Blotto A containing a 1:10,000 dilution of peroxidase conjugated anti-rabbit secondary antibody (Amersham, Arlington Heights, Ill.). After washing in TBS-T, ECL was performed according to the manufacturer's recommendation. Bcl-family proteins expression as determined by Western blot analysis was quantitatively analyzed via laser-scanning densitometry using NIH Image Version 1.61 software. All results were normalized to EF-1α protein expression.

Radiosensitization Assay. U87MG and U251MG cells were plated at 10⁴ cells/well of a 12 well plate (Fisher Scientific) and cultured as described above. Twenty-four hours after plating, cells were infected at an m.o.i. of 50 pfu/cell (based on the initial plating of 10⁴ cells). This is probably an over-estimate of the pfu/cell. Alternatively, cells were treated with either purified GST or GST-MDA-7 (0.25 μg/ml, final concentration) in media. Twenty-four h after infection or protein treatment, cells were exposed to radiation or mock irradiated (6 Gy), at a dose rate of 2.1 Gy/min using a Picker ⁶⁰Co source. Four days after irradiation cells were processed to determine: (a) their growth via MTT assays and (b) their viability by Wright Giemsa staining of fixed cells (Carter et al., 1998, Oncogene 16:2787-2796; Vrana et al., 1999, Radiat. Res. 151:559-569; Dai et al., 2001, Cancer Res. 61:5106-5115).

Wright Giemsa Staining of Cells to Determine Apoptosis. Cells were isolated 96 h after irradiation by trypsinization followed by centrifugation onto glass slides (cytospin). The slides were fixed and stained in Diff-Quik Stain Set (Dade Diagnostics of P.P. Inc, Aguada, PR, USA), according to the manufacturer's instructions, and evaluated under a light microscope for the presence of the classical morphological features of apoptosis (Rosato et al., 2001, Intl. J. Oncology 19:181-191). For each condition, five to ten randomly selected fields of fixed cells were examined (at least ˜150 cells per field, n=5-10 per slide).

MTT Assay to Determine Cell Growth. Cells were grown in 12 well plates and 96 h after irradiation prepared for MTT assays. A 5-mg/ml stock solution of MTT reagent (3-[4,5-Dimethylthiazol-2-yl]-2,5-diphenyltetrazolium bromide; thiazolyl blue) was prepared in DMEM. For assay of mitochondrial dehydrogenase function, the MTT stock solution is diluted 1:10 in fresh media (DMEM+10% fetal calf serum) and 1 ml of this solution is added to each aspirated well of a 12 well plate. Cells are incubated for a further 3 h at 37° C. MTT is converted into an insoluble purple formazan by cleavage of the tetrazolium ring by mitochondrial dehydrogenase enzymes. After 3 h, media is aspirated and cells lysed with 1 ml DMSO, releasing the purple product from the cells. Cells are incubated for a further 10 min at 37° C. with gentle shaking. Absorbance readings at 540 nM are determined using a computer controlled micro-plate analyzer. The relationship between cell number and MTT absorbance/mitochondrial enzyme activity was linear over the range of 10⁴-10⁶ cells.

Results

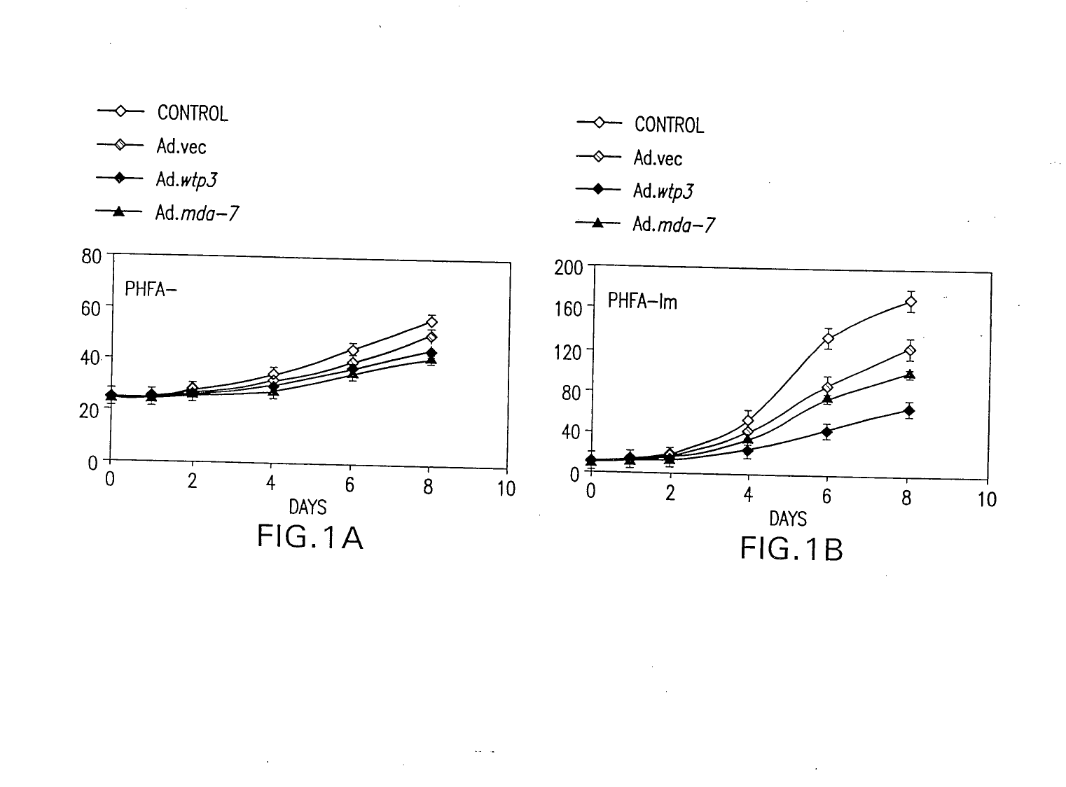

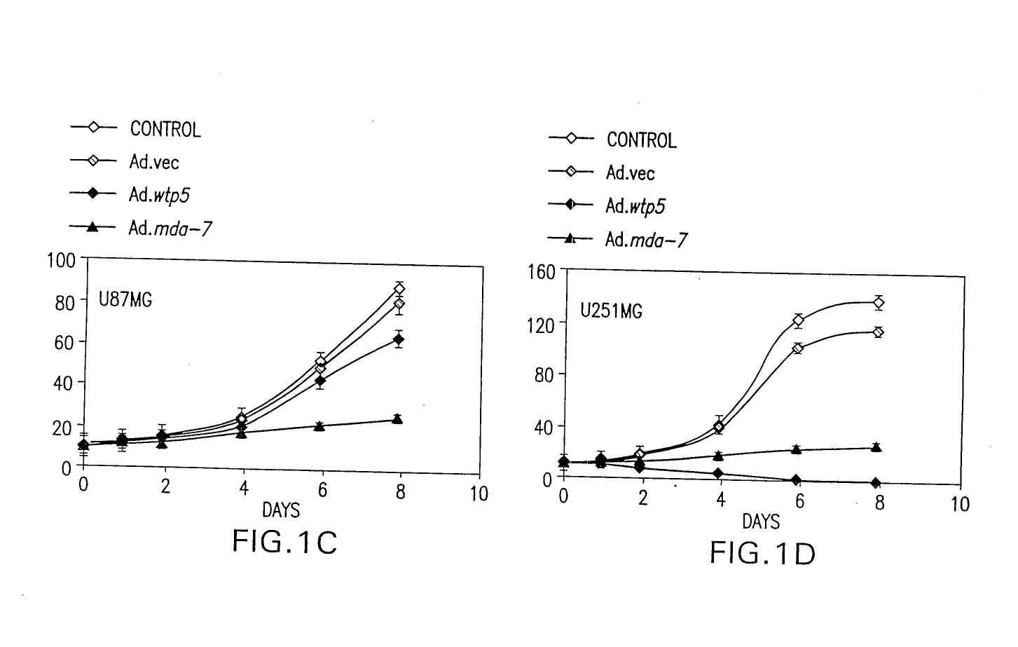

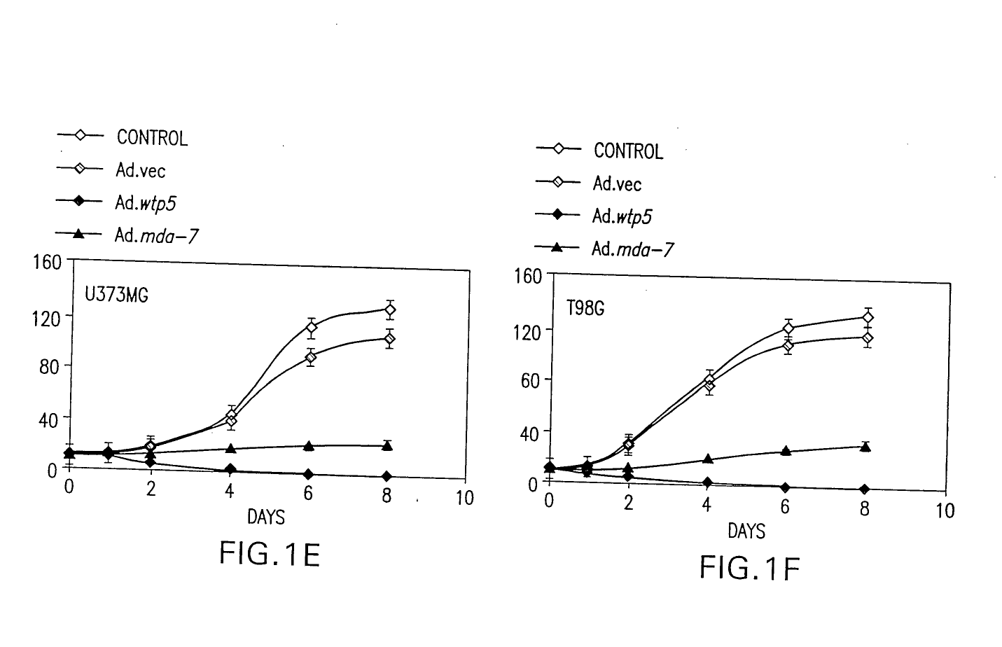

Effect of adenovirus administered mda-7 and wtp53 on growth and transgene expression in PHFA, PHFA-Im and malignant gliomas. The effect of Ad.mda-7 and Ad.wtp53 on growth of early passage (#4) PHFA and PHFA-Im (>25 passages in culture), and wtp53, U87MG, and mutp53, U251MG, U373MG and T98G, human gliomas is shown in FIG. 1. Infection of early passage normal human fetal astrocytes with either virus resulted in modest growth suppression, without decreasing viability. In the case of H-TERT-immortalized PHFA, PHFA-Im, increased growth suppression was observed versus early passage PHFA following infection with Ad.mda-7 or Ad.wtp53. In contrast, infection of both wtp53 and mutp53 gliomas with Ad.mda-7 resulted in enhanced growth suppression (FIG. 1). Although quantitatively more effective in reducing growth than mda-7 in mutp53 gliomas, Ad.wtp53 induced significantly less growth inhibition in U87MG cells than observed following infection with Ad.mda-7 (FIG. 1).

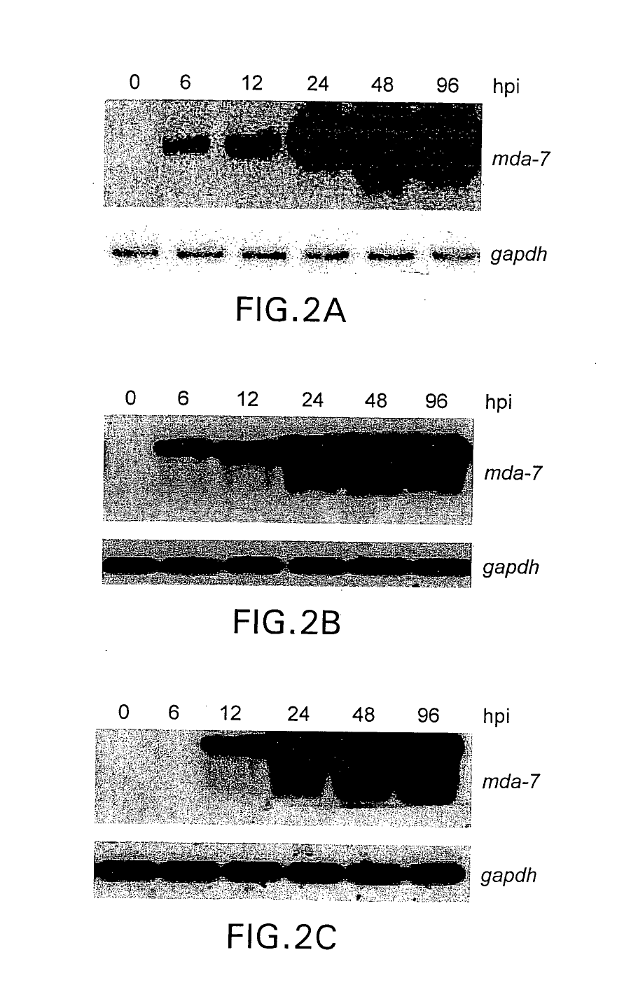

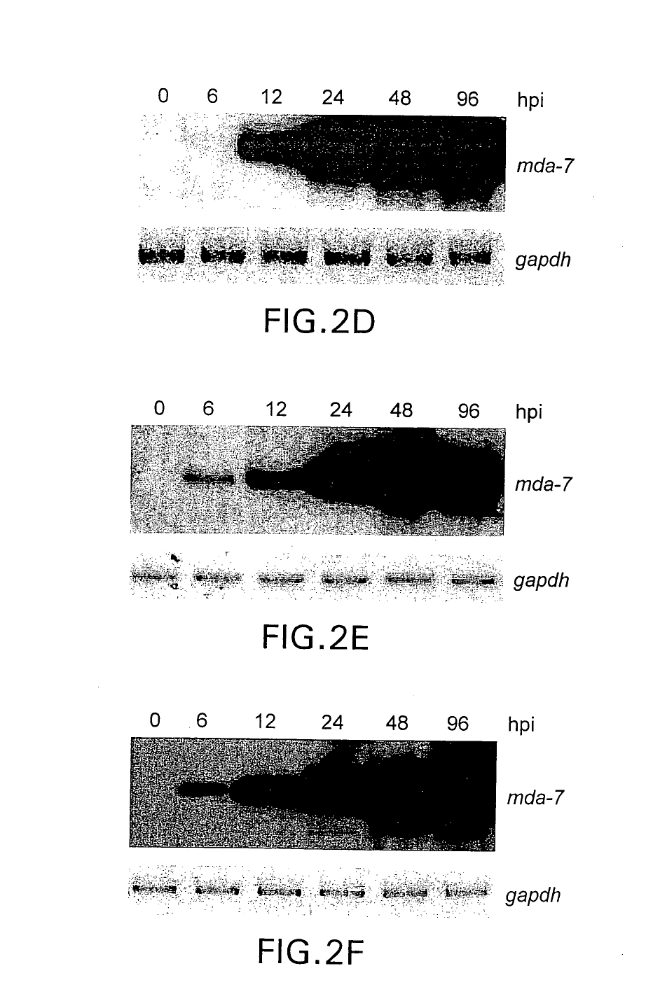

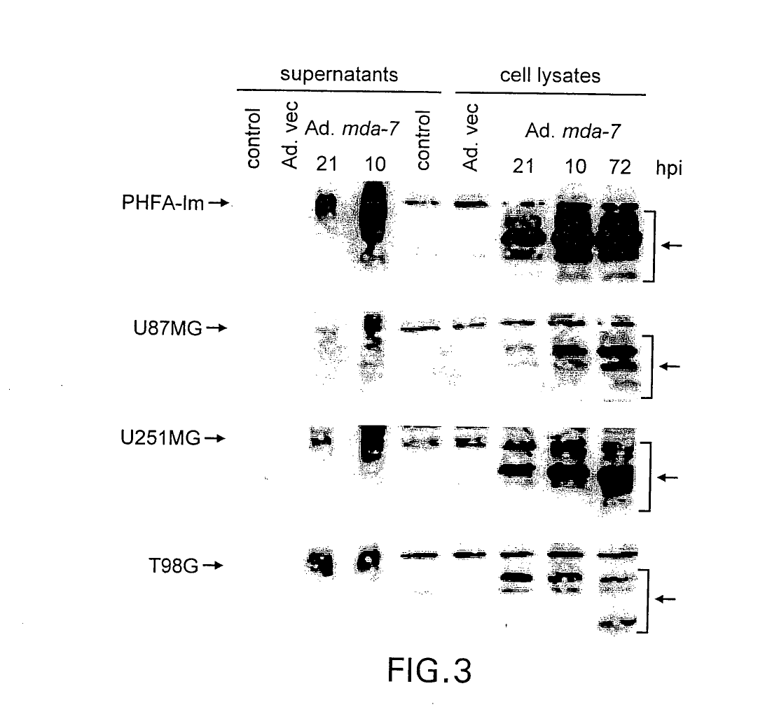

Experiments were performed to define a potential mechanism underlying the differential effect of mda-7 on the growth of normal astrocytes versus malignant gliomas. Infection of PHFA and PHFA-Im with 100 pfu/cell of Ad.mda-7 resulted in readily detectable mda-7 mRNA six h post-infection (hpi), with maximum expression evident by 48 hpi (FIGS. 2A, B). In contrast, mda-7 mRNA was detected by 12 hpi in U87MG and U251MG cells with maximum expression by 96 hpi (FIGS. 2C, D). As observed in normal astrocytes, 6 hpi with 100 pfu/cell of Ad.mda-7 resulted in mda-7 mRNA in U373MG and T98G cells (FIGS. 2E, F). These data document that the differential effect of mda-7 on normal astrocytes versus malignant gliomas is not a consequence of differential expression of the mda-7 transgene in normal cells as compared with their cancerous counterparts. Analysis of coxsackie-adenovirus receptors (CAR), which mediate adenovirus attachment and uptake in cells (Hidaka et al., 1999, J. Clin. Invest. 103:579-587; Li et al., 1999; Pearson et al., 1999, Clin. Cancer Res. 5: 4208-4213; Fechner et al., 2000, Gene Ther. 7: 1954-1968), by FACS analysis indicated the following order relative to numbers of and binding to CAR receptors, T98G>U373MG>U251MG>PHFA>U87MG>PHFA-Im (Table 1). Since U87MG cells are inhibited to a greater extent than PHFA or PHFA-Im and expression of mda-7 is observed more rapidly in PHFA and PHFA-Im than in U87MG or U251MG cells, a simple relationship between virus uptake, transgene expression and differential growth inhibition is not apparent. This conclusion is further supported by analysis of levels of intracellular and secreted MDA-7 protein in PHFA-Im versus the malignant gliomas (FIG. 3). Infection of PHFA-Im and U87MG, U251MG and T98G cells with 100 pfu/cell of Ad.mda-7 resulted in a temporal increase in intracellular and secreted MDA-7 protein. Maximum MDA-7 protein expression, both intracellular and secreted, was apparent in Ad.mda-7 infected PHFA-Im cells. As observed previously, multiple intracellular protein species were detected using MDA-7 specific polyclonal antibodies and higher molecular weight MDA-7 proteins were found in the supernatant of Ad.mda-7 infected normal astrocytes and gliomas (FIG. 3). These results imply, as previously observed in other cell types (Mhashilkar et al., 2001, Mol. Med. 7:271-282; Lebedeva et al., 2002, Oncogene 21:708-718), that the MDA-7 protein is processed prior to secretion and this processing could involve changes in the extent of phosphorylation and/or glycosylation of the secreted proteins. Based on these results it can be concluded that the differential response of normal astrocytes versus the malignant gliomas to mda-7 is not simply a consequence of reduced Ad uptake, intracellular mda-7 transgene expression or production or secretion of MDA-7 protein by normal astrocytes.

TABLE 1
Determination of CAR receptors in normal astrocytes
and glioma cells using monoclonal anti-CAR antibodies
and flow cytometry assay

| | PHFAP#4 | PHFA-Im | U87MG | U373MG | U251MG | T98G |
|---|---|---|---|---|---|---|
| Peak | 1.74 | 0.07 | 0.17 | 6.43 | 9.68 | 10.04 |
| shift | | | | | | |
| D | 0.37 | 0.07 | 0.1 | 0.85 | 0.79 | 0.91 |

Peak shift is calculated as a ratio (P_{CAR} = P_{control})/P_{control}, where P is the median of the fluorescent peak of the FACS histogram. D value represents the differences between the two curves (Kolmogorov-Smirnov test); higher numbers reflect enhanced binding and increased CAR availability.

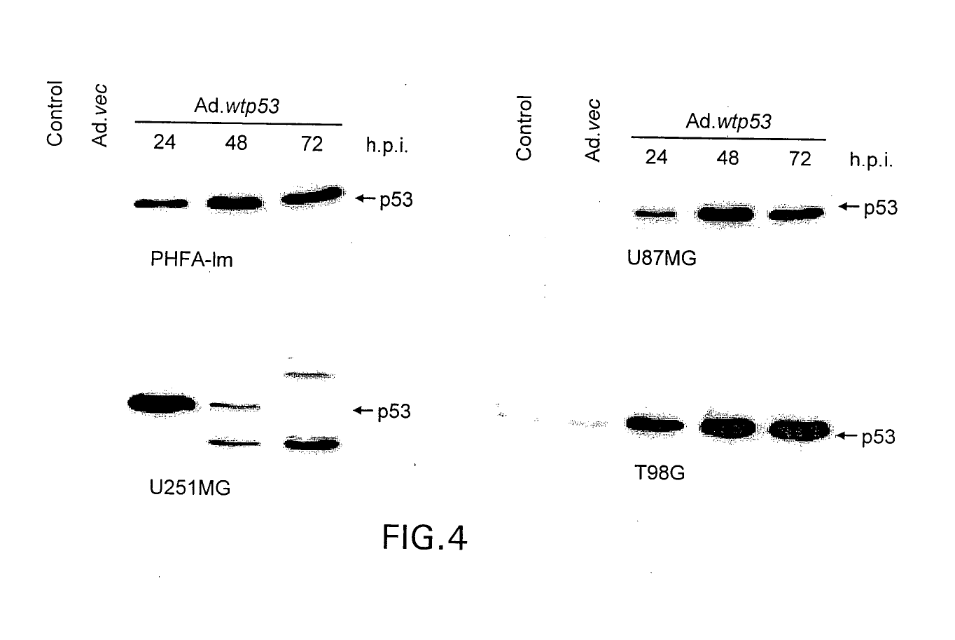

Previous studies document that Ad.wtp53 differenfially affects gliomas, depending on their p53 genotype (Cirielli et al., 1999, J. Neuro-Oncology 43: 99-108; Lang et al, 1999, Neurosurgery 45: 1093-1104; Hong et al., 2000, J. Korean Med. Sci. 15: 315-322). The present study confirms a selective effect of wtp53 on the growth of mutp53 human gliomas (FIG. 1). Western blotting analysis using a p53 monoclonal antibody, which detects both wtp53 and mutp53, indicates elevated p53 protein in PHFA-Im, mutp53 (U251MG and T98G) and wtp53 (U87MG) cells after infection with Ad.wtp53 (FIG. 4). In the case of U251MG cells, levels of p53 protein decrease during the course of infection, and a lower molecular weight protein is detected, which may represent a p53 degradation product. These results support the conclusion that differential growth suppression in PHFA-Im and U87MG cells versus U251MG and T98G cells following infection with Ad.wtp53 is not a consequence of failure to infect these cells or express the p53 transgene.

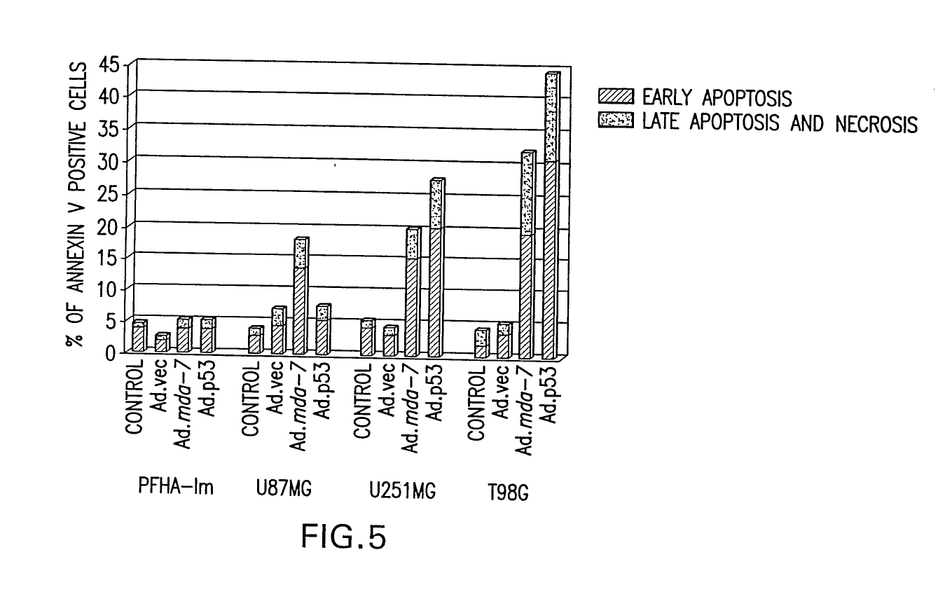

Ad.mda-7 induces apoptosis selectively in malignant gliomas. Infection of diverse human cancers, but not normal cells, with Ad.mda-7 results in a loss in viability by induction of programmed cell death (apoptosis) (Su et al., 1998, Proc. Natl. Acad. Sci. U.S.A. 95:14400-14405; Su et al., 2001, Proc. Natl. Acad. Sci. U.S.A. 98:10332-10337; Madireddi et al., 2000, Adv. Exp. Med. Biol. 465:239-261; Saeki et al., 2000, Gene Ther. 7:2051-2057; Mhashilkar et al., 2001, Mol. Med. 7:271-282; Lebedeva et al., 2002, Oncogene 21:708-718; Paeter et al., 2002; Saeki et al., 2002, Oncogene 21: 4558-4566; Sarkar et al., 2002, Proc. Natl. Acad. Sci. U.S.A. 99:10054-10059). Annexin V staining and FACS analysis demonstrated that infection with 100 pfu/cell of Ad.mda-7 increased the percentage of early apoptotic cells in U87MG, U251MG and T98G cells, and elevated the levels of late apoptotic and necrotic cells, most markedly in T98G cells (FIG. 5). In contrast, no significant change in the percentage of apoptotic cells was evident in gliomas infected with 100 pfu/cell of Ad.vec (adenovirus lacking the transgene insert). Infection of PHFA-Im with 100 pfu/cell of Ad.mda-7 or Ad.vec also did not significantly change the percentage of early or late apoptotic (or necrotic) cells. In the case of Ad.wtp53, both early and late apoptotic (or necrotic) U251MG and T98G cells were increased after infection with 100 pfu/cell, but apoptosis was not induced in PHFA-Im or wtp53 U87MG cells (FIG. 5).

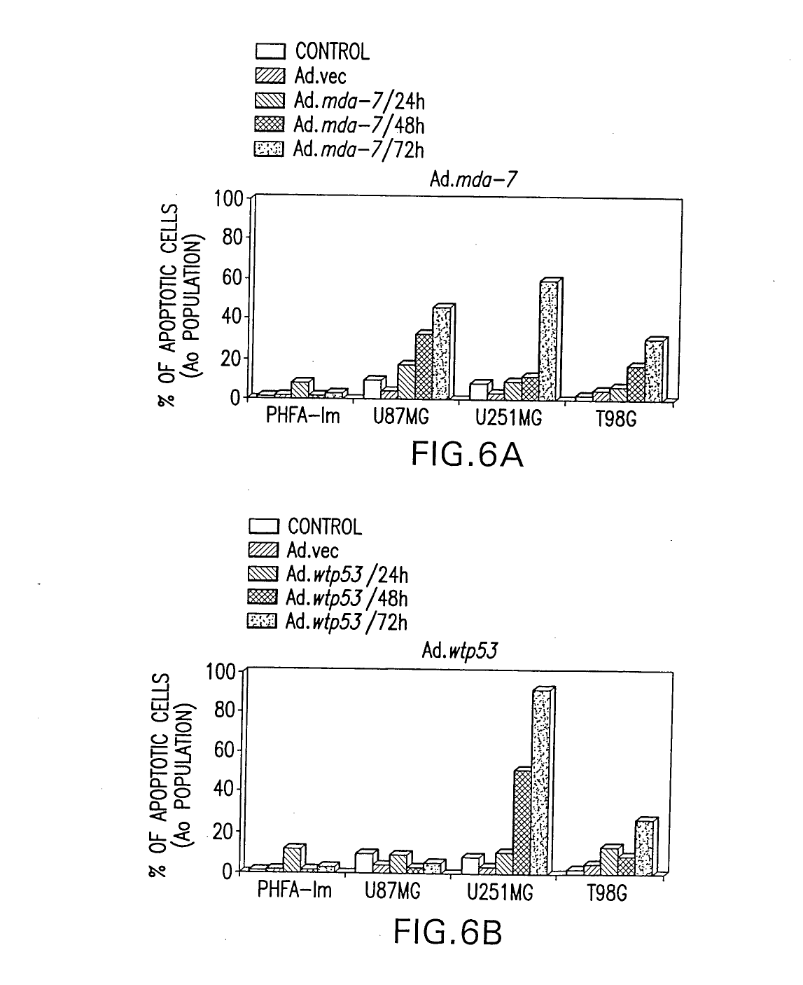

Cell cycle analysis after infection with Ad.mda-7 or Ad.wtp53 provided additional support for induction of apoptosis in specific gliomas by these viruses (FIG. 6). In the case of Ad.mda-7, a temporal increase in the proportion of cells with a sub G₀/G₁ (A₀) DNA content was evident in U87MG, U251MG and T98G cells (FIG. 6). At day 3 after infection with 100 pfu/cell of Ad.mda-7, ˜59%, ˜46% and ˜31% of U251MG, U87MG and T98G cells, respectively, contained an A₀ DNA content. In the case of Ad.wtp53, an increase in A₀ DNA content was apparent in mutp53 U251MG (˜93%; at day 3) and T98G (˜27%; at day 3) cells, but not in wtp53 U87MG cells (FIG. 6). In contrast, except for a small increase in A₀ DNA content 24 h after Ad.mda-7 or Ad.wtp53 infection, only control baseline A₀ DNA content was evident in PHFA-Im 48 or 72 hpi with the different viruses. Similarly, analysis of DNA fragmentation using the TUNEL reaction and FACS analysis indicated induction of apoptosis by Ad.mda-7 in glioma cells, U87MG (˜44%; day 2), U251MG (˜62%; day 2) and T98G (˜69%; day 2), but not in PHFA-Im (˜1%; day 2).

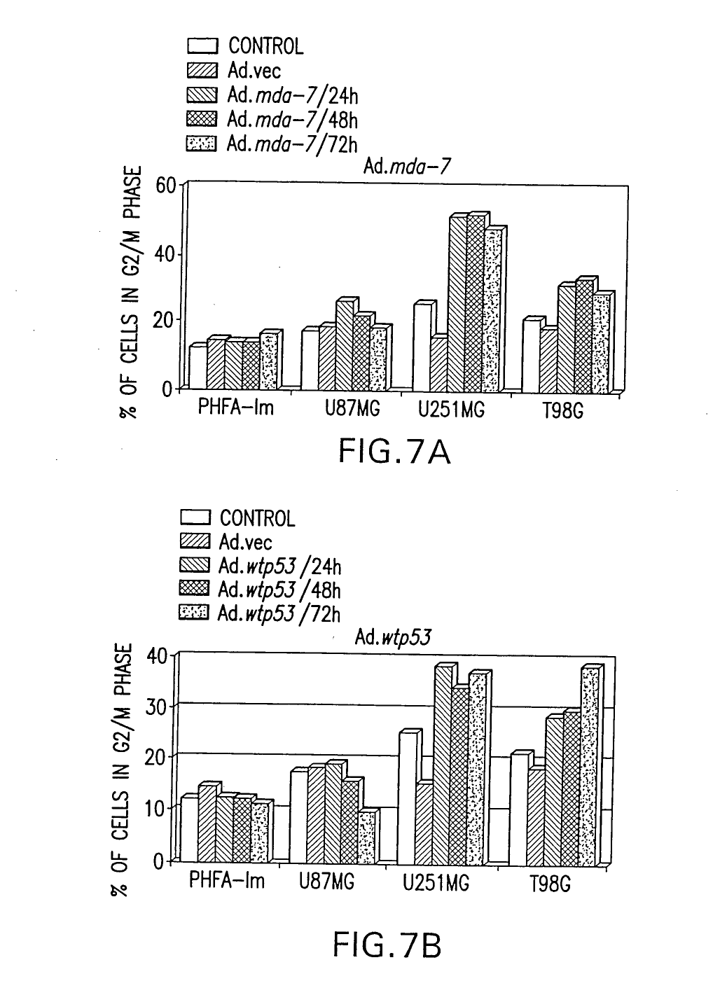

Previous studies reveal that Ad.mda-7 infection can induce an increase in the percentage of specific cancer cell types in the G₂/M phase of the cell cycle (Saeki et al., 2000, Gene Ther. 7:2051-2057; Mhashilkar et al., 2001, Mol. Med. 7:271-282; Lebedeva et al., 2002, Oncogene 21:708-718). Infection of U87MG, U251MG and T98G gliomas with Ad.mda-7 increased the percentage of cells in the G₂/M phase of the cell cycle by 24 hpi, with no further increase after 48 or 72 hpi (FIG. 7). The magnitude of this cell cycle effect was greatest in U251MG cells. In contrast, only a marginal increase (<2%) in cells in the G₂/M phase of the cell cycle was apparent after Ad.mda-7 infection of PHFA-Im (FIG. 7). In the case of Ad.wtp53, an increase in cells in the G₂/M phase of the cell cycle was apparent in both U251MG and T98G cells, but not in U87MG cells or PHFA-Im (FIG. 7). These results provide additional support for a selective effect of Ad.mda-7 on cell cycle in human gliomas, which is independent of p53 genotype. In contrast, Ad.wtp53 induces cell cycle alterations only in human gliomas that have a mutp53 genotype.

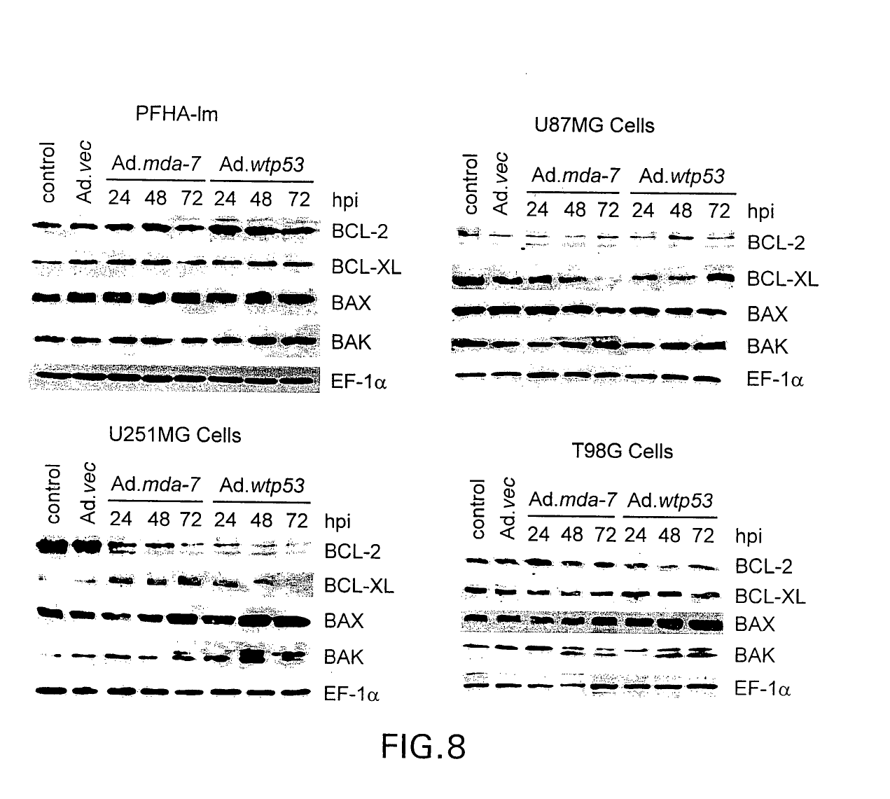

Ad.mda-7 alters levels of proapoptotic and antiapoptotic proteins selectively in malignant gliomas. Infection of diverse cancer cells with Ad.mda-7 results in an elevation in the levels of BAX protein and alters the ratio of BAX to Bcl-2 proteins (Su et al., 1998, Proc. Natl. Acad. Sci. U.S.A. 95:14400-14405; Madireddi et al., 2000, Adv. Exp. Med. Biol. 465:239-261; Mhashilkar et al., 2001, Mol. Med. 7:271-282; Su et al., 2001, Proc. Natl. Acad. Sci. U.S.A. 98:10332-10337; Lebedeva et al., 2002, Oncogene 21:708-718). Studies in human melanoma also indicate changes in the levels of additional proapoptotic proteins, such as BAK, and antiapoptotic proteins, such as Bcl-XL, in specific melanomas, but not in normal melanocytes, following infection with Ad.mda-7 (Lebedeva et al., 2002, Oncogene 21:708-718). Based on these considerations, the temporal effect of infection with 100 pfu/cell of Ad.mda-7, Ad.wtp53 or Ad.vec on the levels of antiapoptotic proteins, Bcl-2 and Bcl-XL, and proapoptotic proteins, BAX and BAK, were determined by Western blotting (FIG. 8 and Table 2). In the case of PHFA-Im, no consistent or significant changes were evident in the different apoptosis associated proteins after 24, 48 or 72 hpi with any of the viruses. In U87MG cells, the levels of Bcl-2 and Bcl-XL proteins were decreased by 48 h following Ad.mda-7 infection, whereas this effect was not apparent following Ad.wtp53 infection (FIG. 8 and Table 2). In these cells, no significant changes were evident in the levels of BAX or BAK following infection with either virus. In mutp53 U251MG cells, a decrease in Bcl-2 and increases in both BAX and BAK were apparent after infection with Ad.mda-7 or Ad.wtp53 (FIG. 8 and Table 2). In T98G cells, an ˜1.5- to ˜1.9-fold decrease in Bcl-2 and Bcl-XL proteins and an ˜1.4 to ˜1.7-fold increase in BAX and BAK proteins occurred after infection with Ad.mda-7 or Ad.wtp53. These results are consistent with a mechanism in which decreases in the levels of anti-apoptotic (Bcl-2/Bcl-XL) versus pro-apoptotic (BAX/BAK) proteins resulting from infection with Ad.mda-7 act as primary mediators of induction of apoptosis in specific cancer cells. In PHFA-Im, the minor changes induced in these protein subtypes following Ad.mda-7 would be anticipated to nullify each other, thereby not affecting cell survival. Similarly, in the case of U87MG cells, infection with Ad.wtp53 did not alter these critical apoptosis associated proteins and programmed cell death was not induced.

TABLE 2
Effect of Ad.mda-7 and Ad.wtp53 on pro- and antiapoptotic Bcl-family
proteins in normal immortal astrocytes and malignant gliomas

| | PHFA-Im | | U87MG | | U251MG | | T98G | |
|---|---|---|---|---|---|---|---|---|
| | Ad.mda-7 | Ad.wtp53 | Ad.mda-7 | Ad.wtp53 | Ad.mda-7 | Ad.wtp53 | Ad.mda-7 | Ad.wtp53 |
| Bcl-2 | 1.1↓ | 1.4↑ | 2.5↓ | 1.2↑ | 3.2↓ | 7.1↓ | 1.5↓ | 1.6↓ |
| Bcl-xL | 1.1↑ | 1.4↑ | 8.4↓ | No change | 1.4↑ | 1.1↓ | 1.9↓ | 1.5↓ |
| BAX | 1.4↑ | 1.4↑ | 1.1↓ | 1.3↓ | 2.5↑ | 2.5↑ | 1.4↑ | 1.7↑ |
| BAK | 1.2↑ | 1.2↑ | 1.3↑ | 1.2↑ | 5↑ | 10↑ | 1.6↑ | 1.7↑ |

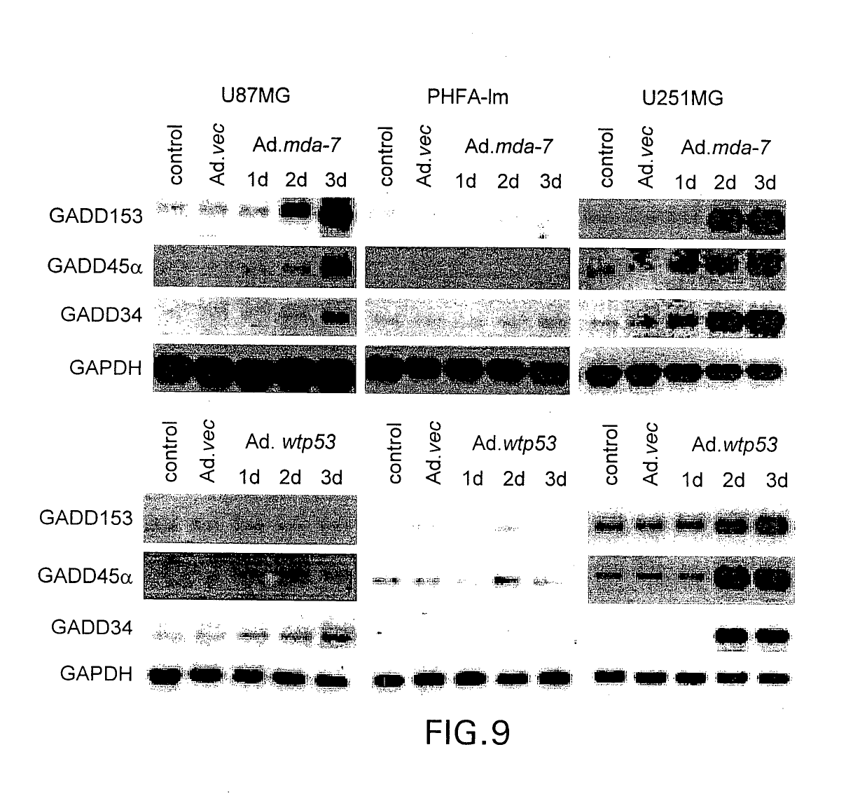

Ad.mda-7 induces GADD gene expression in malignant gliomas. Ad.mda-7 appears to execute its apoptosis-inducing effect by inducing growth arrest and DNA damage-inducible (GADD) genes in melanoma cell lines (Sarkar et al., 2002, Proc. Natl. Acad. Sci. U.S.A. 99:10054-10059). The present experiments were designed to determine whether the GADD family of genes were also upregulated in malignant gliomas following Ad.mda-7 infection. Since the GADD family of genes is also modulated by p53, the effect of Ad.wtp53 infection on GADD gene expression in PHFA-Im and in wtp53 and mutp53 malignant gliomas was also investigated. PHFA-Im, wtp53 U87MG cells and mutp53 U251MG cells were infected with 100 pfu/cell of Ad.vec, Ad.mda-7 or Ad.wtp53 and the expression pattern of the GADD family of mRNAs was examined by Northern blot analysis 1 to 3 days postinfection. As shown in FIG. 9, Ad.mda-7 infection, but not Ad.vec infection, induced GADD153, GADD45α and GADD34 mRNAs in a time-dependent manner in both U87MG and U251MG cells, which were susceptible to the killing effect of Ad.mda-7 (FIGS. 5, 6, 7). In both cases, the induction of GADD 153 was significantly higher than that of the other GADD genes. However, Ad.mda-7 infection did not induce these genes in PHFA-Im, which are resistant to the apoptosis-inducing effects of Ad.mda-7 (FIGS. 5, 6, 7). Ad.wtp53 infection induced the GADD family of genes only in U251MG cells, which are killed by Ad.wtp53, but not in PHFA-Im or U87MG cells, which are not killed by Ad.wtp53. These differential expression patterns of the GADD family of genes by Ad.mda-7 and Ad.wtp53 demonstrate an advantage of Ad.mda-7 relative to Ad.wtp53 in the context of gliomas, in that Ad.mda-7 was observed to induce expression of GADD genes in glioma cells irrespective of their p53 status.

Ad.mda-7 infection and GST-MDA-7 protein sensitizes malignant glioma cells to radiation-induced growth suppression and induction of apoptosis. Mda-7 expression results in accumulation of specific glioma cells in the G₂/M phase of the cell cycle, and since the G₂/M phase is known to be the most radiosensitive portion of the cycle, it seemed logical to next determine whether there was any growth-suppressive interaction between exposure of cells to ionizing radiation and expression of mda-7. U87MG and U251MG cells were plated and 24 h later infected with Ad.mda-7 or Ad.vec at an m.o.i. of 50 pfu/cell. Cells were cultured for an additional 24 h prior to irradiation. Cultures were then irradiated (0-8 Gy) and growth was determined 4 days later (5 days after infection) using MTT assays. Parallel experiments examined the amount of apoptosis 4 days after exposure.

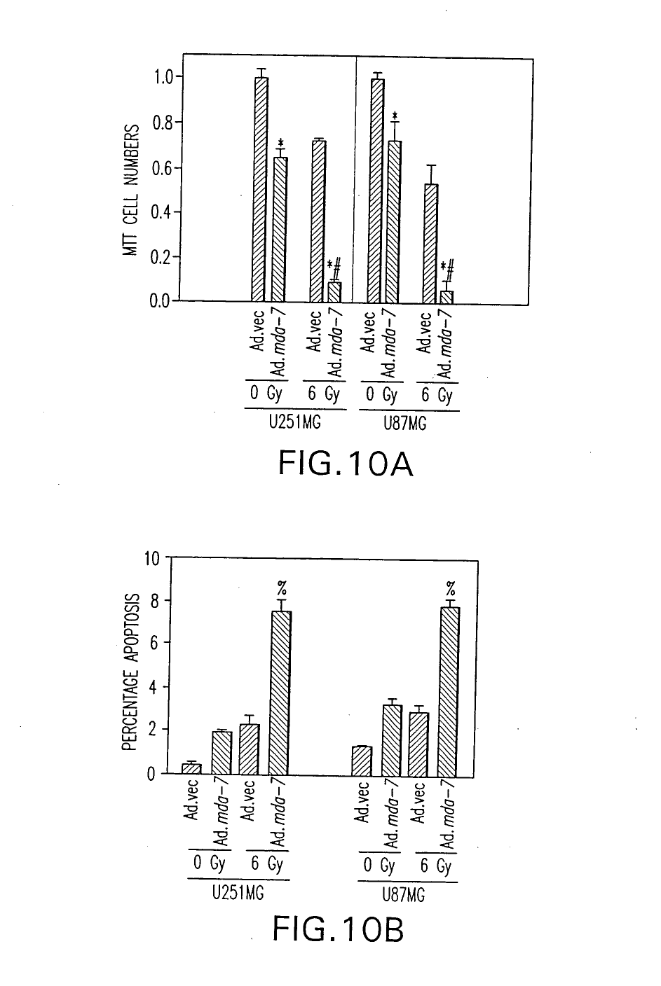

Expression of mda-7 or irradiation of U87MG and U251MG glioma cells reduced their growth potential. However, the combination of mda-7 and ionizing radiation caused a statistically significant additional decrease in growth potential of gliomas (FIG. 10A). In agreement with data in FIGS. 5 and 6, mda-7 increased the numbers of apoptotic cells, as judged by Wright Giemsa staining for apoptotic cell morphology, which was significantly enhanced following radiation exposure (FIG. 10B). This data suggests that a part of the mechanism by which radiation enhances the anti-proliferative effects of mda-7 is by increasing apoptosis.

Because MDA-7 is a secreted protein, now renamed IL-24 (Kotenko, 2002, Cytokine and Growth Factor Rev. 217: 1-18; Sarkar et al., 2002, Biotechniques October:30-39; Cytokine Growth Factor Rev. 14:35-51), additional studies were performed to assess the effect of MDA-7 protein on tumor cell growth and the impact of purified MDA-7 on cellular radiosensitivity. Culture media of primary rat hepatocytes infected with Ad.mda-7, but not control virus, suppressed the growth of prostate carcinoma cells. A bacterial expression system was developed wherein mda-7 was fused to glutathione-S-transferase, to prepare larger quantities of pure MDA-7 protein for further studies (GST-MDA-7). To determine whether purified MDA-7 protein altered cell growth and survival, gliomas were treated with either vehicle (GST) or MDA-7 (GST-MDA-7) (0.25 μg/ml final concentration in media), followed 24 h later by exposure to ionizing radiation. Cell growth was determined 4 days after irradiation using MTT assays.

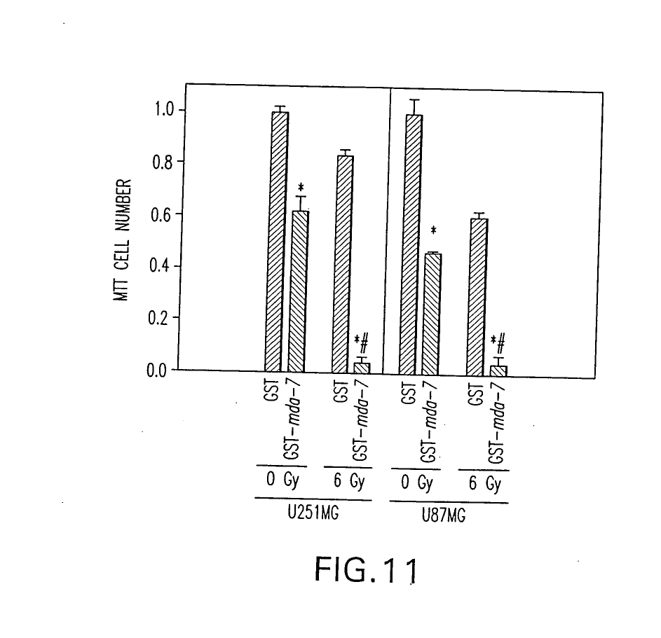

Treatment with GST-MDA-7 protein or irradiation of U87MG and U251MG gliomas reduced their growth potential. However, the combination of GST-MDA-7 and ionizing radiation caused a statistically significant additional decrease in growth potential of the gliomas (FIG. 11). GST-MDA-7 also potentiated radiation-induced apoptosis in gliomas that was qualitatively similar to that resulting from Ad.mda-7 (FIG. 10B). Collectively, this data demonstrates that mda-7 can enhance the anti-proliferative and cytotoxic actions of ionizing radiation.

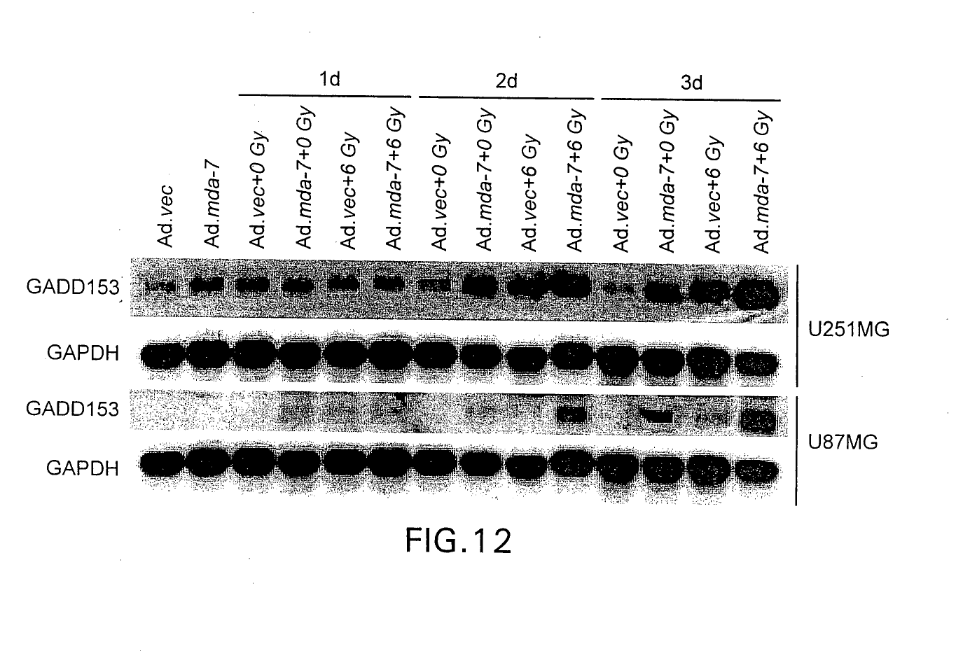

Ad.mda-7 plus radiation results in enhanced induction of the GADD family of genes in malignant glioma cells. UV and ionizing radiation are known to cause induction of the GADD family genes, which can play a role in apoptotic processes (Hollander et al., 1997, J. Biol. Chem. 272: 13731-13737; Amundson et al., 1998, Oncogene 17: 2149-2154; Carrier et al., 1998, Biochem. Pharmacol. 55: 853-861). Since Ad.mda-7 infection and GST-MDA-7 facilitated the killing effect of ionizing radiation in malignant gliomas (FIGS. 10, 11) it was determined whether the expression pattern of the GADD family of genes was modulated by the combined treatment of ionizing radiation and Ad.mda-7. As shown in FIG. 12, infection of U251MG and U87MG cells with Ad.mda-7 at an m.o.i. of 50 pfu/cell, or exposure of cells to ionizing radiation (6 Gy), induced GADD153 expression from day 2 of treatment onward. However, combined treatment of cells with both radiation and mda-7 further augmented the induction of GADD153, thus providing a possible mechanism underlying the cooperative effect of Ad.mda-7 and ionizing radiation in killing malignant gliomas.

### 6.2 Example 2

### mda-7 Inhibits Growth and Enhances Radiosensitivity of Glioma Cells In Vitro and In Vivo

Materials and Methods

Reagents. Dulbecco's Modified Eagle's Medium (DMEM) and Penicillin-Streptomycin were from Gibco (Life Technology, New York). MTT reagent (3-[4,5-Dimethylthiazol-2-yl]-2,5-diphenyltetrazolium bromide), and Giemsa Stain were from Sigma (St. Louis, Mo.). Anti-Caspase 3, Anti-Bcl-2, Anti-Bcl-XL, Anti-F AS receptor, Anti-F AS ligand, Anti-BAX and all the secondary antibodies (anti-rabbit-HRP, anti-mouse-HRP, and anti-goat-HRP) were purchased from Santa Cruz Biotechnology (Santa Cruz, Calif.). Anti-P ARP (1:2500, mouse monoclonal Calbiochem). Enhanced chemiluminescence (ECL) kit was purchased from NEN Life Science Products (NEN Life Science Products, Boston, Mass.). Other plasmid constructs and reagents were as described in (Su et al., 1998, Proc. Natl. Acad. Sci. U.S.A. 95:14400-14405; Dent et al., 1999, Mol. Biol. Cell 10:2493-2456; Hagan et al., 2000, Radiat. Res. 153:371-383; Su et al., 2001, Proc. Natl. Acad. Sci. U.S.A. 98:10332-10337; Yacoub et al., 2001, J. Radiat. Biol. 77:1067-1078; Lebedeva et al., 2002, Oncogene 21:708-718; Sarkar et al., 2002, Proc. Natl. Acad. Sci. U.S.A. 99:10054-10059).

Generation of Ad.mda-7 and Synthesis of GST-MDA-7. Recombinant type 5 adenoviruses to express MDA-7 (Ad.mda-7), control (CMV vector) or control (β-galactosidase) were generated using recombination in HEK293 cells as described (Hitt et al., 1997, Adv, Pharmacol. 40:137-206; Dent et al., 1999, Mol. Biol. Cell. 10:2493-2506). Standard cloning procedures were used to generate a bacterial expression vector comprising in-frame fusion of the mda-7 ORF 3′ to the GST ORF in GST-4T2 vector (Amersham Pharmacia), using BamHI and NotI sites introduced into mda-7 by PCR (Su et al., 2003 Oncogene 22:1164-1180). Expression of protein was performed by inoculating an overnight culture at 1:100 dilution followed by incubation at 25° C. until an O.D.₆₀₀ of 0.4-0.6 was reached followed by induction with 0.1 μM IPTG for 2 h. Cells were harvested by centrifugation and sonicated in PBS followed by centrifugation to obtain soluble protein. The lysate was bound to a glutathione-agarose column (Amersham Pharmacia) at 4° C. for 2 h followed by washing with 50 volumes PBS and 10 volumes PBS with 500 mM NaCl. Elution of bound protein was performed by passing 20 mM reduced glutathione through the column and collecting 1 ml fractions. Fractions were analyzed by gel electrophoresis and positive samples were dialyzed against 1000 volumes of PBS for 4 h with one change followed by 500 volumes of DMEM for 4 h. Protein concentration was estimated by Bradford assays as well as gel electrophoresis in conjunction with Coomassie blue staining. Samples were tested for activity using GST protein as control. Using gel-purified GST-MDA-7, a polyclonal anti-GST-MDA-7 antibody was raised in rabbits and used at a 1:3000 dilution for immunoblotting.

Cell Culture. Fischer 344 rat RT2 gliomablastoma cells (University of Alabama, Birmingham) and primary rodent astrocytes (kindly provided by Dr. E. Ellis, Dept. Pharmacology and Toxicology, V.C.U.) were cultured in DMEM supplemented with 10% fetal bovine serum (Hyclone, Logan, Utah) and 1% penicillin/streptomycin. Cells were incubated in humidified atmosphere of 5% CO₂ at 37° C.

Recombinant Adenovirus Infection. The Ad.mda-7 and control adenoviral vectors used were identical to those described previously (Su et al., 1998, Proc. Natl. Acad. Sci. U.S.A. 95:14400-14405; Su et al., 2001, Proc. Natl. Acad. Sci. U.S.A. 98:10332-10337; Lebedeva et al., 2002, Oncogene 21:708-718). The viral titers for each adenovirus and infection efficiency for each cell type were determined by plaque formation assay. In vitro adenoviral infections were performed 24 h after plating (Volkert and Young, 1983, Virology 125: 175-193). Monolayer cultures were washed in PBS and incubated with purified virus in 1 ml of growth medium without serum for 1 h at 37° C. in a humidified atmosphere of 5% CO₂/95% air with gentle agitation. After 3 h, fresh growth medium with 10% fetal bovine serum was added.

Assessment of Apoptosis and Cell Viability. The extent of apoptosis was evaluated by assessing Wright-Giemsa stained cytospin slides under light microscopy and scoring the number of cells exhibiting the classic morphological features of apoptosis. For each condition, 10 randomly selected fields per slide were evaluated, encompassing at least 15000 cells (Grant et al., 1996, Exp. Cell. Res. 228:65-75). To confirm the results of morphologic analysis, in some cases cells were also evaluated by TUNEL staining and oligonucleosomal DNA fragmentation assay as follows; staining, cytospin slides were fixed with 4% formaldehyde/PBS for 10 min, treated with acetic acid/ethanol (1:2) for 5 min, and incubated with terminal transferase reaction mixture containing IX terminal transferase reaction buffer, 0.25 U/I terminal transferase, 2.5 mM CoCl₂, and 2 pmol fluorescein-12-dUTP (Boehringer Mannheim, Indianapolis, Ind.) at 37° C. for 1 h. The slides were mounted with Vectashield containing propidium iodide (Vector Laboratories, Burlingame, Calif.) and visualized using fluorescence microscopy (Dai et al., 2002, Cell Cycle 1: 143-152).

Assessment of Cell Viability. Cell viability was also evaluated by assessing trypan blue inclusion/exclusion of isolated cells under light microscopy and scoring the percentage of cells exhibiting blue staining (Grant et al., 1996, Exp. Cell. Res. 228:65-75). Floating and attached cells were isolated by trypsinization, recovered by centrifugation, resuspended in phenol red free DMEM and mixed 1:1 with trypan blue reagent. Cells (˜400) were counted in all four fields of a hemocytometer.

MIT Assay for Determination of Cellular Viability. The MTT test is based on the enzymatic reduction of the tetrazolium salt MTT in living, metabolically active cells. Cells were plated (5-10,000 cells per well of a 12 well plate) and 24 h after plating infected with either Ad.mda-7 or control virus at the indicated multiplicity of infection (m.o.i.). In other experiments, cells were plated (5-10,000 cells per well of a 12 well plate) and 24 h after plating treated with either GST or GST-MDA-7 at the indicated concentrations. Twenty-four hours after infection/protein treatment, cells were treated with kinase inhibitor drugs and then irradiated. The cytotoxicity of the various treatments was assessed four days after irradiation by measurement of cell viability by use of the MTT assay, as described previously (Mosmann, 1983, J. Immunol. Methods 65:55-63). The plates were read on a Dynatech MR600 Microplate Reader at 540 nm. All data were normalized relative to the control, non-treated unirradiated cells of the corresponding cell type.

Cell Survival Analyses. Cells were assayed for the effect(s) of Ad.mda-7, and radiation on cell survival. Cells were plated (10,000 cells per 60 mm dish) and 24 h after plating infected with either Ad.mda-7 or control virus at the indicated m.o.i. Twenty four hours after infection cells were irradiated. Ninety-six hours after irradiation, cells were isolated by trypsinization and viable trypan blue negative cells re-plated in 60 mm dishes at 250-1,000 cells per plate. Colonies were allowed to form from surviving cells for 7-9 days, before fixing and staining with crystal violet. Colonies that contained more than 50 cells were then counted. To generate the survival data, individual assays were performed at multiple dilutions with a total of four plates per data point.

Western Blot Analysis. Protein concentration was determined using a kit from Bio-Rad. Aliquots (40 μg) were solubilized in Laemmli buffer, separated by SDS-PAGE, and transferred to nitrocellulose membranes as described. Membranes were blocked 2 hours at 4° C. in TBST (5% nonfat milk in 10 mM Tris/HCl, 100 mM NaCl, and 0.1% Tween-20, pH 7.6). Membranes were exposed to the primary antibodies, followed by washing (3×15 min with TBST). The following antibodies were used: mouse anti-Bcl-_{XL}, BAX, and Bcl-2 monoclonal antibody; mouse anti-PARP, anti-beta-actin (Santa Cruz Biotechnology, CA); anti-p53 (polyclonal antibody (Oncogene Research Products, Cambridge, Mass.); mouse anti-Fas antibody (Pharmigen, San Diego, Calif.). Membranes were incubated with horseradish peroxidase-conjugated anti-mouse or anti-rabbit IgG antibody, followed by washing with TBST (3×15 min). Proteins were visualized by ECL and quantified by densitometry.

DNA Fragmentation. Equal number of cells from each test sample (106) were homogenized with 1 ml lysis buffer (10 mM Tris at pH 7.4, 5 mM EDTA, 1% Triton X-100). RNase A 100 μg/ml was added to each sample and incubated at 50° C. for 1 hour. Proteinase K was then added (100 μg/ml) and the samples were incubated overnight at 50° C. The DNA was extracted using phenol and chloroform, and centrifuged at 10,000×g for 5 min at 4° C. The aqueous phase was mixed with 2 volumes of ice-cold ethanol and then precipitated by centrifugation at 15,000×g for 10 min. Supernatants were removed, and DNA pellets were washed with 80% ethanol once (15,000 times; g for 10 min), air-dried, dissolved in TE buffer at pH 7.6. DNA concentration were determined and 10 μg of each sample was then electrophoresed on a 1.5% agarose gel and analyzed for the presence of a laddering pattern.

Statistical Analyses. Comparison of the effects of various treatments was performed using one way analysis of variance and a two tailed t-test. Differences with a p-value of <0.05 were considered statistically significant. For animal studies, a log rank test was applied to data. Experiments shown are the means of multiple individual points (±SEM).

Results

Ad.mda-7 enhances the radiosensitivity of RT2 as measured in MIT assays. RT2 cells were infected with increasing amounts of Ad.mda-7 or control virus and the expression of MDA-7 determined 48 h after infection. Increasing the viral particle multiplicity of infection (m.o.i.) enhanced the amount of MDA-7 protein produced in each cell.

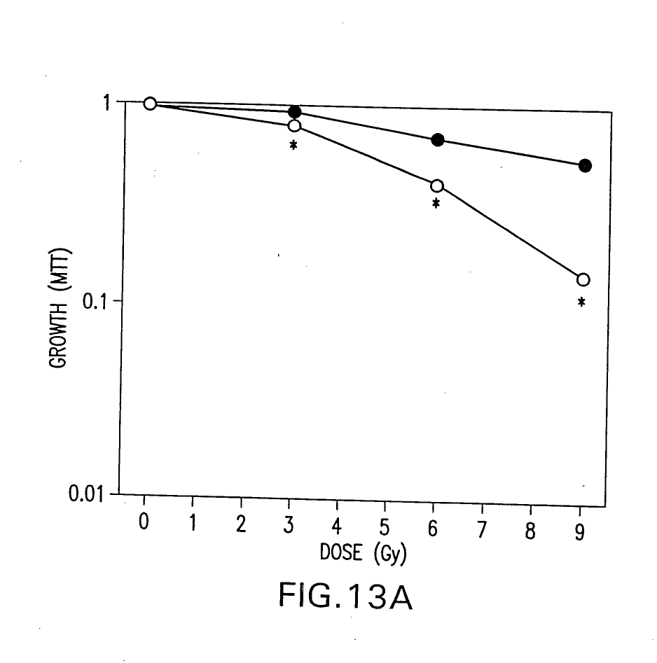

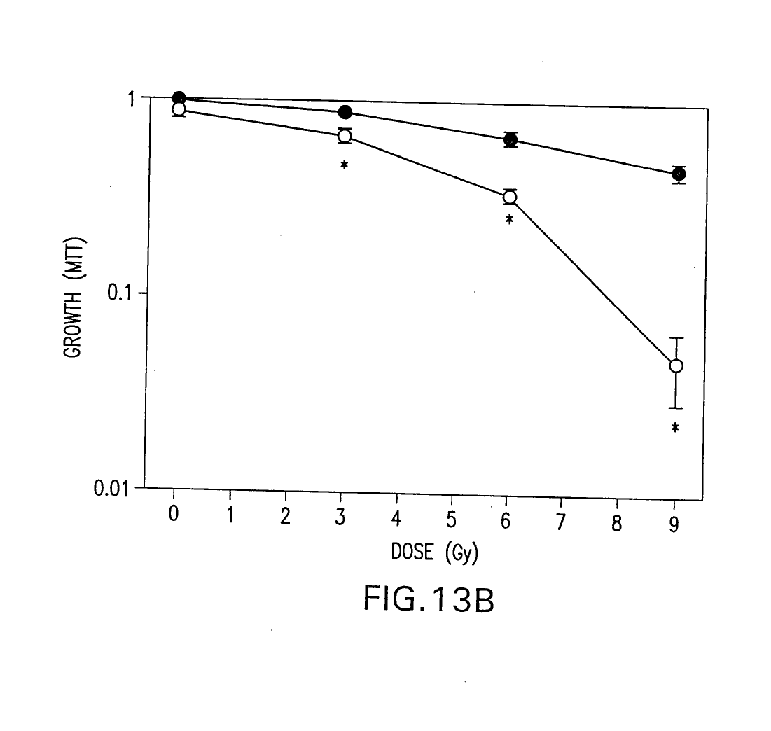

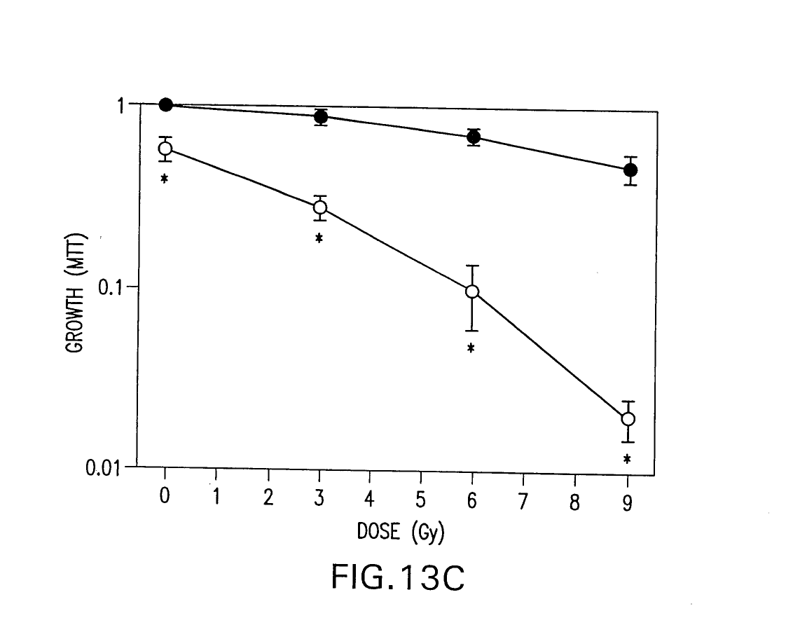

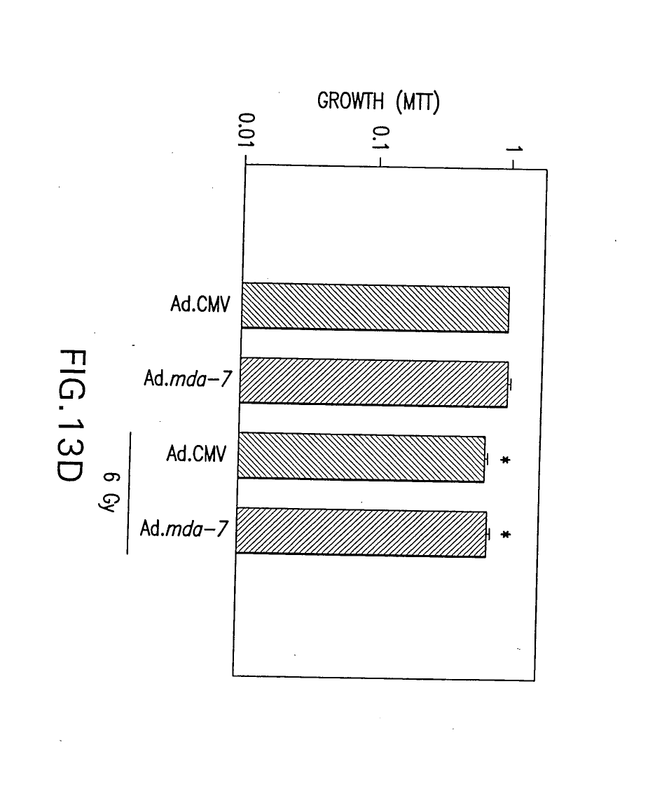

Previous studies have shown that infection of tumor cells, but not non-transformed cells, with Ad.mda-7 inhibited tumor cell growth (Su et al., 1998, Proc. Natl. Acad. Sci. U.S.A. 95:14400-14405; Su et al., 2001, Proc. Natl. Acad. Sci. U.S.A. 98:10332-10337, Su et al., 2003 Oncogene 22:1164-1180; Madireddi et al., 2000, Adv. Exp. Med. Biol. 465:239-261; Saeki et al., 2000, Gene Ther. 7:2051-2057; Saeki et al., 2002, Oncogene 21:4558-4566; Mhashilkar et al., 2001, Mol. Med. 7:271-282; Ellerhorst et al., 2002, J. Clin. Oncol. 20:1069-1074; Lebedeva et al., 2002, Oncogene 21:708-718; Sarkar et al., 2002, Biotechniques October:30-39; Sarkar et al., 2002, Proc. Natl. Acad. Sci. U.S.A. 99:10054-10059; Sauane et al., 2003, J. Cell. Physiol. 196:334-345). To assess the effect of Ad.mda-7 on the growth and survival of RT2 glioma cells, cells were assayed for proliferation via MTT assay and viability via trypan blue staining after Ad.mda-7 infection and radiation exposure. Cells were plated, infected and irradiated 24 h after infection with increasing radiation doses (FIGS. 13A-13D). Increasing the viral m.o.i. resulted in a dose-dependent reduction in glioma cell growth. Furthermore while radiation reduced proliferation, it interacted with Ad.mda-7, but not control virus, to further reduce cell growth. These effects were not observed in either primary rodent astrocytes (FIG. 13D) or primary human astrocytes (Su et al., 2003, Oncogene 22:1164-1180).

In general agreement with the findings in FIG. 13, Ad.mda-7 enhanced cell death, as judged by trypan blue staining, that was significantly increased following radiation exposure (Table 3). These effects were not observed in primary rodent astrocytes (Table 3), in which cell survival, as measured by nuclear apoptotic morphology, was more weakly enhanced by the Ad.mda-7+radiation combination. Expression of MDA-7 (Ad.mda-7 infected at 25 m.o.i.) or irradiation enhanced the levels of apoptotic cells from 0.7±0.1% to 1.8±0.2% and 1.3±0.1%, respectively. However, combined treatment of cells with Ad.mda-7 and radiation increased the percentage of apoptotic cells to 5.0±0.4% (p<0.05 greater than the combined effects of Ad.mda-7 and radiation individually).

TABLE 3
Ad.mda-7 causes a dose-dependent increase in glioma cell death
that is enhanced in a greater than additive fashion by ionizing
radiation. Cells were cultured for 24 h then infected with Ad.mda-7
or CMV control viruses (5, 25 or 50 multiplicity of infection
(m.o.i.)) as described in Materials and Methods. The cells were
irradiated (6 Gy) 24 hours after infection. Cells were isolated 96 h
after irradiation and cell viability determined by trypan blue
exclusion staining. Data are the means ± SEM of 3-4 separate
experiments: *p < 0.05 greater than control infected cells;
^{#}p < 0.05 greater than irradiated cells, corrected
for the toxic effects of Ad.mda-7.

| | Percentage of trypan blue-positive cells | | | |
|---|---|---|---|---|
| Cell type | | | Ad.CMV + | Ad.mda-7 + |
| (m.o.i.) | Ad.CMV | Ad.mda-7 | 6 Gy | 6 Gy |
| RT2 | 3.3 ± 1.7 | 7.0 ± 1.8 | 6.3 ± 1.7 | 18.1 ± 3.4*^{#} |
| (5) | | | | |
| RT2 | 3.8 ± 1.6 | 11.9 ± 2.5* | 8.0 ± 3.2 | 33.9 ± 3.8*^{#} |
| (25) | | | | |
| RT2 | 3.7 ± 1.8 | 18.7 ± 1.2* | 6.2 ± 2.3 | 47.4 ± 3.3*^{#} |
| (50) | | | | |
| 1° | 3.7 ± 0.9 | 3.6 ± 1.1 | 7.9 ± 2.1 | 8.5 ± 2.1* |
| Astrocyte | | | | |
| (50) | | | | |

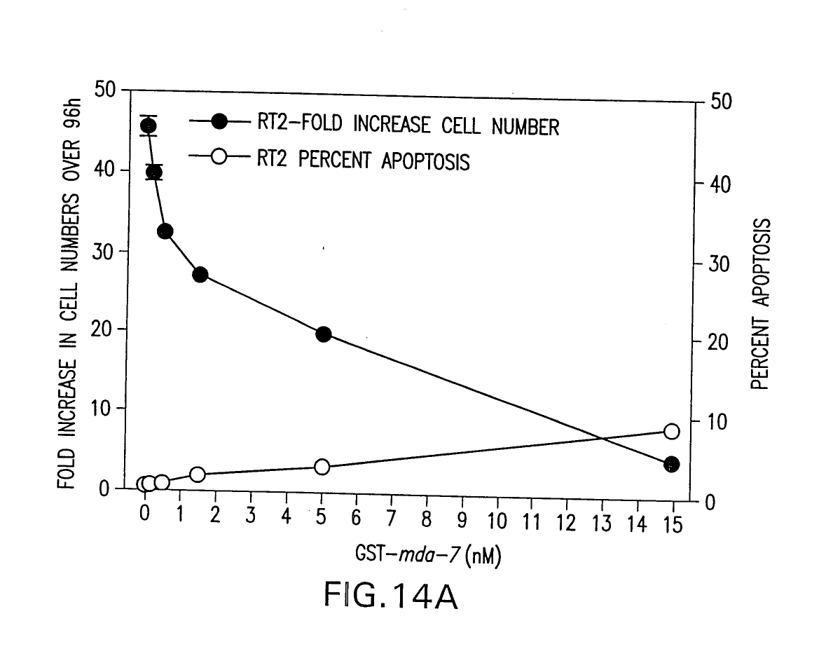

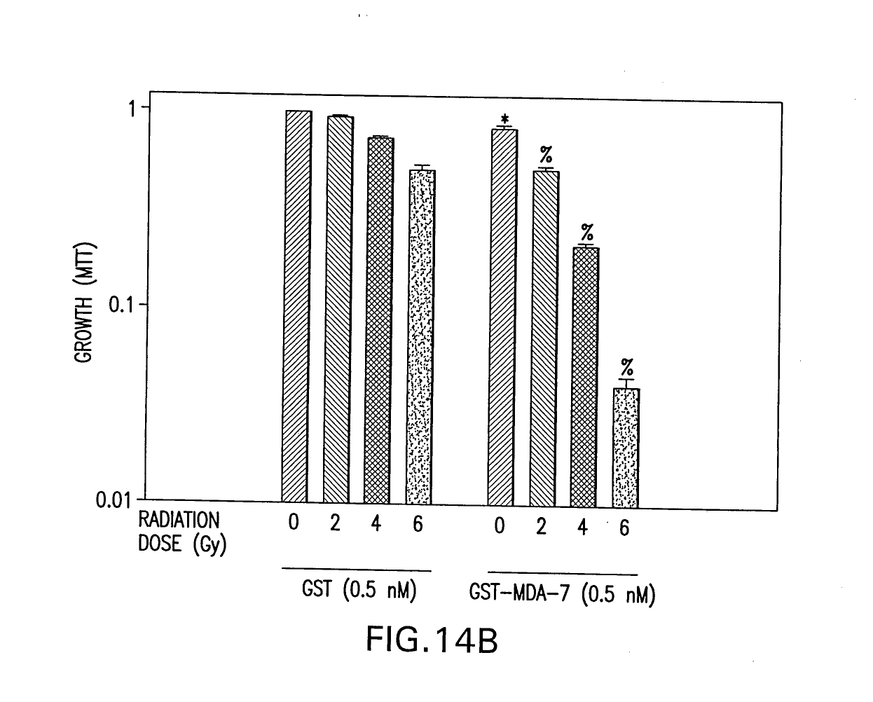

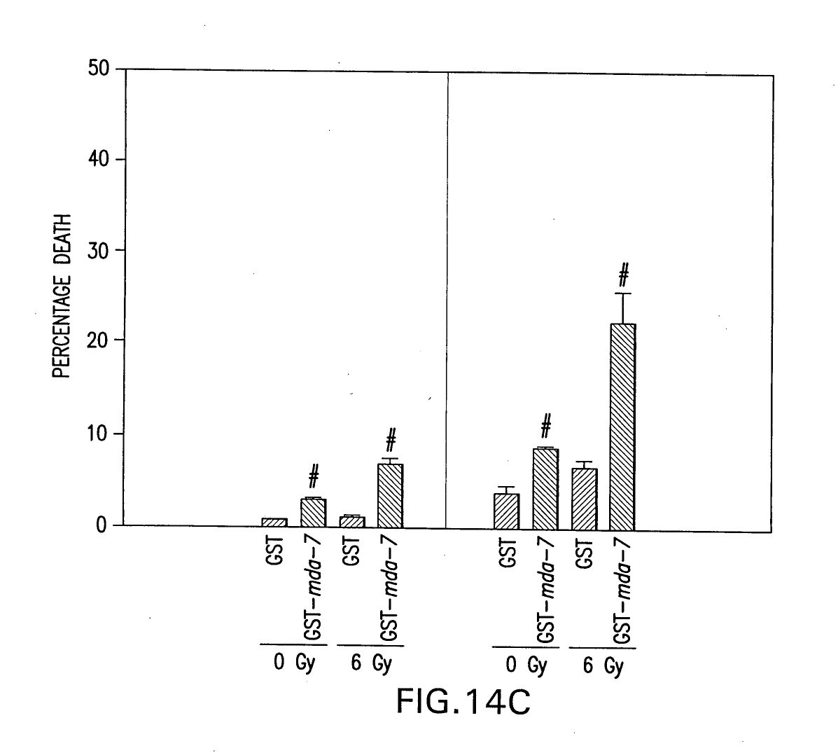

Purified GST-MDA-7 protein has similar growth inhibitory and radiosensitizing effects as Ad.mda-7. To confirm that the findings in FIG. 13 and Table 3 were not due to an effect related to viral infection, RT2 cells were treated with bacterially synthesized GST-MDA-7 or GST (FIG. 14) followed by exposure to ionizing radiation (Su et al., 2003 Oncogene 22:1164-1180). GST-MDA-7, but not GST, caused a dose-dependent reduction in RT2 cell growth (FIG. 14A): however, only at concentrations of GST-MDA-7 above 15 nM was any increase in cell killing by GST-MDA-7 observed. In agreement with data in FIG. 13, GST-MDA-7 suppressed the growth of RT2 cells that was enhanced in a greater than additive fashion by ionizing radiation (FIG. 14B). These findings also correlated with enhanced cell killing as judged by Giemsa staining (FIG. 14C, left set of bars) and by trypan blue staining (FIG. 14C, right set of bars).

Expression of MDA-7 down-regulates expression of the mitochondrial anti-apoptotic molecule Bcl-_{XL}. The Bcl-2 gene family consists of both positive and negative regulators of apoptosis and interactions between these molecules can modulate the apoptotic threshold for a wide variety of noxious stimuli. Two family members in particular, Bcl-2 and Bcl-_{XL}, function as potent inhibitors of cell death, and have been shown to enhance resistance to chemotherapeutic agents and ionizing radiation (Simonian et al., 1997, Blood 90:1208-1216).

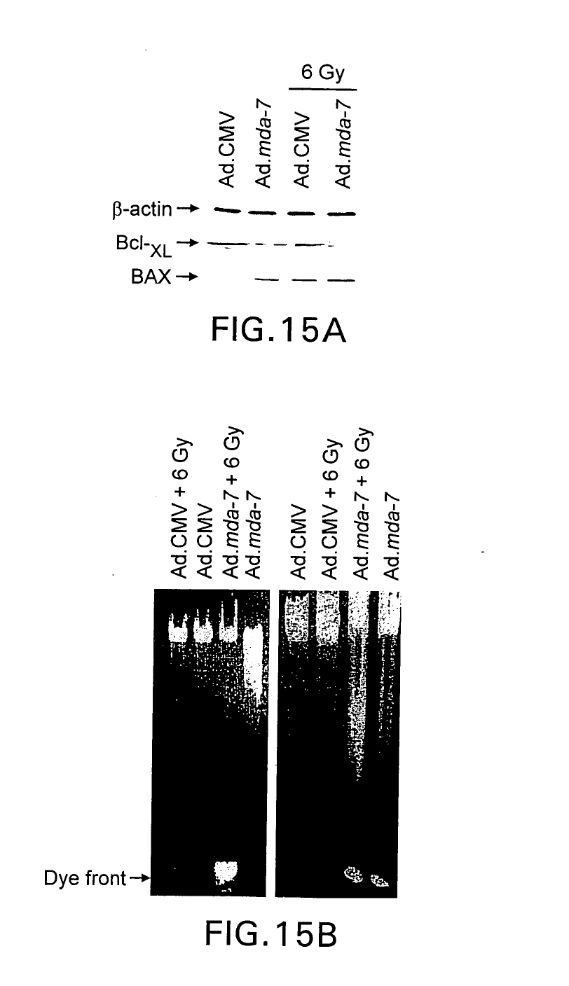

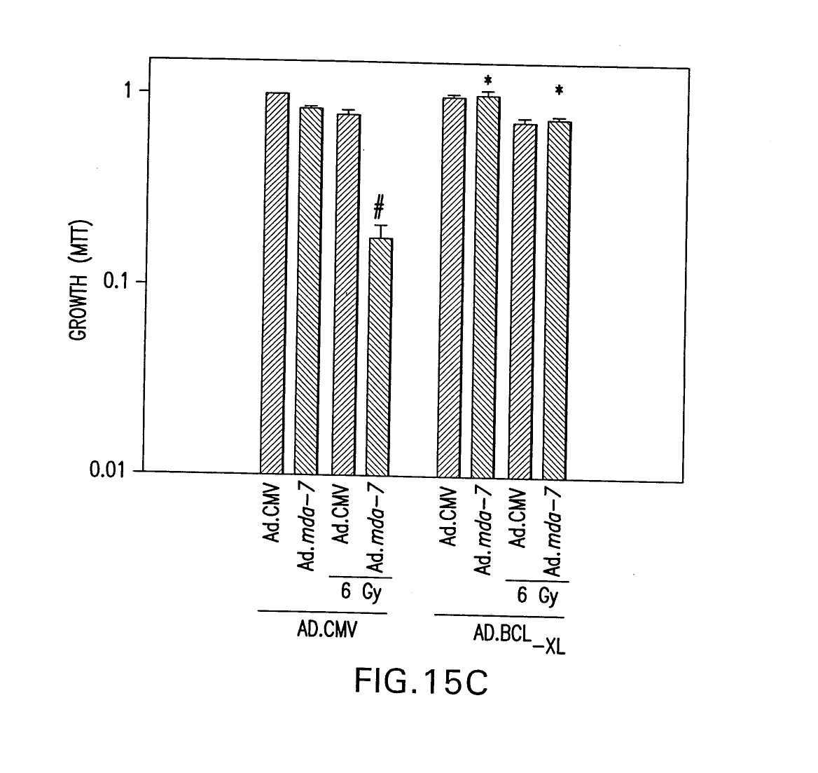

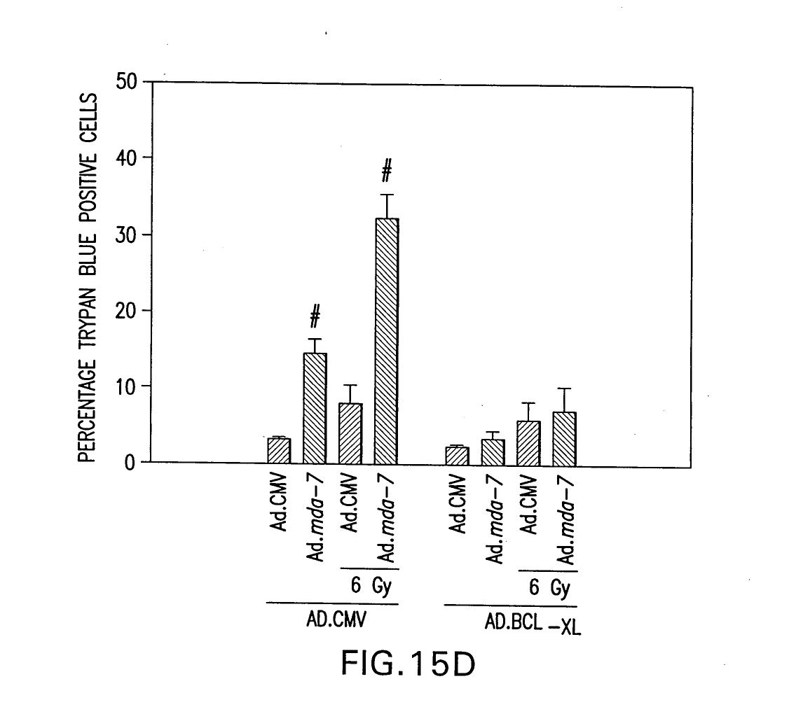

In parallel to studies in FIG. 13 and Table 3, the expression of pro- and anti-apoptotic Bcl-2 family members as well as the levels of FAS and TNFα receptors was determined by immunoblotting. Infection of RT2 cells with Ad.mda-7, but not control virus, reduced expression of Bcl-_{XL} and enhanced expression of BAX (FIG. 15A). Radiation did not alter Bcl-XL expression but also enhanced BAX levels. The combination of Ad.mda-7 and radiation resulted in a further modest increase in BAX expression and a large decrease in Bcl-XL levels. Little alteration was observed in the expression of Bcl-2, as well as the FAS and TNFα death receptors or their ligands. The reduction in Bcl-_{XL} levels and enhancement in BAX expression also correlated with a reduction in p32 pro-caspase 3 levels and cleavage of PARP.

As an additional separate measure of cell survival, the integrity of nuclear DNA, 96 h after irradiation/20 h after viral infection was examined. Irradiation and/or infection of cells with control virus did not significantly alter DNA integrity (FIG. 15B, two separate studies shown). However, infection of cells with Ad.mda-7 caused the appearance of a smear of lower molecular weight DNA as well as the appearance of low molecular weight DNA fragments that electrophoresed at or near the dye front, suggestive of necrosis. Irradiation of cells exhibiting Ad.mda-7-induced DNA degradation resulted in a large decrease in high molecular weight nucleosomal DNA and an increase in amount of the low molecular weight DNA fragments.

To test whether altered Bcl-_{XL} expression was causal in the modified proliferation and survival of glioma cells exposed to MDA-7 and radiation, RT2 cells were infected with Ad.Bcl-_{XL}, Ad.mda-7 or control virus, and 24 h afterwards irradiated. Cell growth and viability were determined 96 h after irradiation. Constitutive over-expression of Ad.Bcl-_{XL} abrogated the growth inhibitory effect of Ad.mda-7 and Ad.mda-7+radiation (FIG. 15C). Over-expression of Bcl-_{XL} also reduced the anti-proliferative effect of ionizing radiation. These findings correlated with a reduction in cell killing by Ad.mda-7 and Ad.mda-7+radiation (FIG. 15D).

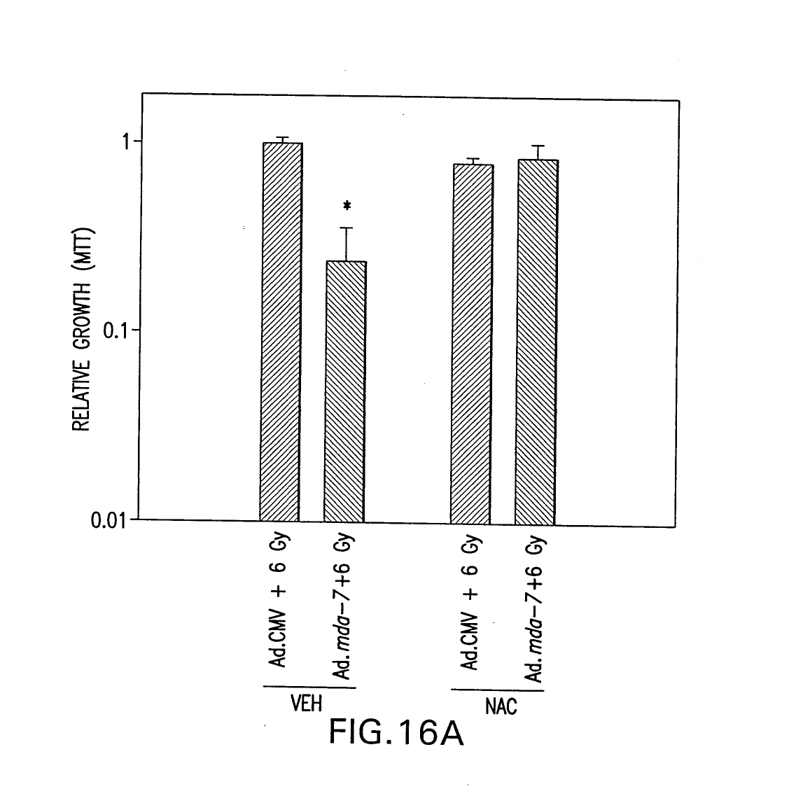

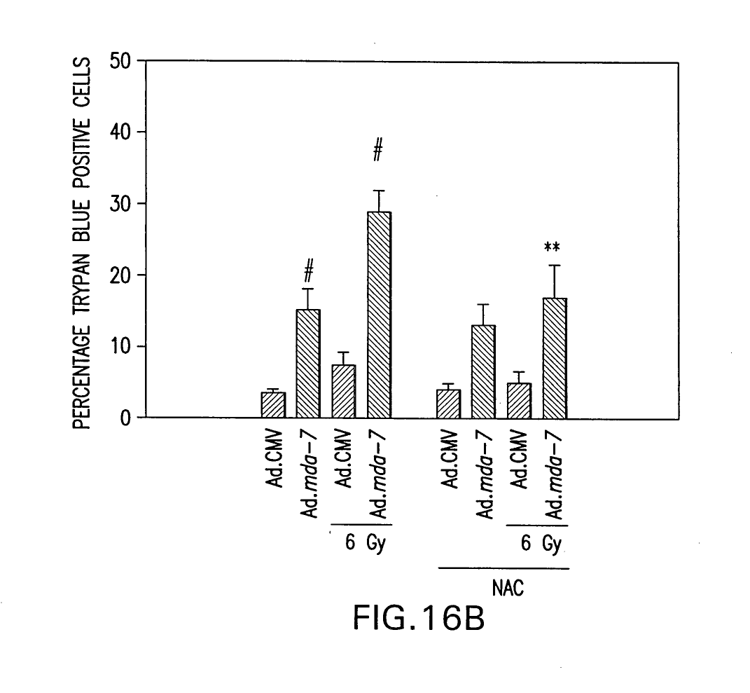

The free radical scavenger N-acetyl-L-cysteine (NAC) blunted the anti-proliferative interaction between Ad.mda-7 and radiation. Ionizing radiation has been shown to enhance the production of mitochondria-derived free radicals that may be linked to altered cell signaling and survival processes (Leach et al., 2001, Cancer Res. 61: 3894-3901). Expression of Bcl-_{XL} can, in part, reduce the generation of free radicals by mitochondria (Brockhaus and Brune, 1999, Oncogene 18:6403-6410; Leach et al., 2001, Cancer Res. 61: 3894-3901). To determine whether radiation-induced free radicals interact with MDA-7 to alter cell viability a free radical-scavenging agent, N-acetyl-L-cysteine, was added to infected RT2 cells prior radiation. NAC abolished the enhancement in cell killing and reduction in proliferation of RT2 cells treated with Ad.mda-7+radiation (FIG. 16A). This finding also correlated with a reduction in cell killing (FIG. 16B).

Ad.mda-7 enhances the radiosensitivity of RT2 cells as measured in colony formation assays. In FIG. 13, short-term MTT growth assays demonstrated that Ad.mda-7 interacted with radiation to cause a greater than additive reduction in proliferation that correlated with increased cell death. Additional studies were performed to determine whether Ad.mda-7 altered glioma cell colony formation after irradiation in vitro. Infection of cells with Ad.mda-7 (5 m.o.i.) weakly reduced the colony formation of cells (0.89±0.07), compared to control viral infection (1.00±0.08), that was not significant. Radiation (6 Gy) caused a significant reduction in cell survival (0.29±0.03). The combination of Ad.mda-7 and radiation reduced colony formation that was significantly greater than the additive effects of either treatment alone (0.02±0.005, P<0.05).

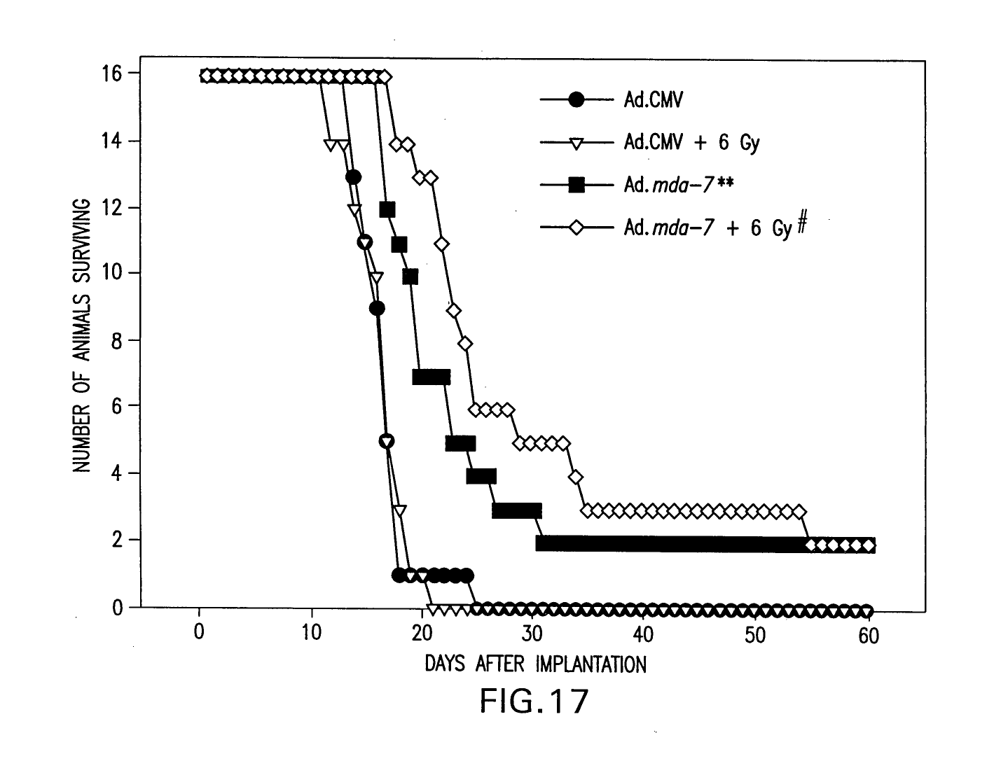

Ad.mda-7 enhances the survival of rats implanted intracranially with RT2 cells. These studies have utilized the RT2 rodent glioma cell line, in part, because it is syngeneic to the Fischer 344 rat (Park et al., 2001, Oncogene 20:3266-3280). RT2 cells were infected with either control virus or Ad.mda-7 in vitro and 24 h after infection, 10⁴ cells implanted into the brains of Fischer 344 rats. Four days after implantation the head of each rat was irradiated (6 Gy). The survival of the rats was noted on a daily basis. Rats implanted with control virus infected cells, regardless of whether they were irradiated, died within 15-20 days (FIG. 17). Rats implanted with Ad.mda-7 infected cells survived significantly longer than control virus alone or control virus+radiation animals (** p<0.05). Irradiation of rats implanted with Ad.mda-7 infected cells resulted in a further significant increase in animal survival beyond that of Ad.mda-7 alone (#p<0.05).

### 6.3 Example 3

### mda-7 Inhibits Growth and Enhances Radiosensititivity of Glioma Cells In Vitro Via JNK Signaling

Materials and Methods

Reagents. Dulbecco's Modified Eagle's Medium (DMEM), Minimum Essential Medium Alpha (MEMα) and Penicillin-Streptomycin were from Gibco (Life Technology, New York). Minimum Essential Medium (MEM), Nonessential Amino Acids (NEAA), and Sodium Pyruvate were from Cellgro (VA). The (PD98059) selective MEK 1/2 inhibitor, and (LY294002) the PI3K inhibitor (Calbiochem, La Jolla, Calif.) were made in DMSO and added 60 minutes prior to radiation treatment (Xia et al., 1995, Science 270:1326-1331; Jiang et al., 1996, Proc. Natl. Acad. Sci. U.S.A. 93:9160-9165; Cartee et al., 2000, Int. J. Oncol. 16:413-422; Madireddi et al., 2000, Adv. Exp. Med. Biol. 465:239-261; Mhashilkar et al., 2001, Mol. Med. 7:271-282; Saeki et al, 2002;) MTT reagent (3-[4,5-Dimethylthiazol-2-yl]-2,5-diphenyltetrazolium bromide), and Giemsa Stain were from Sigma (St. Louis, Mo.). Anti-Caspase 3, Phospho-/total-ERK1/2, Phospho-/total-P38α/β, Phospho-/total-JNK1/2, Phospho-/total-AKT, (anti-rabbit-HRP, anti-mouse-HRP, and anti-goat-HRP) were purchased from Santa Cruz Biotechnology (Santa Cruz, Calif.). Anti-P ARP (1:2500, mouse monoclonal Calbiochem). Enhanced chemiluminescence (ECL) kit was purchased from NEN Life Science Products (NEN Life Science Products, Boston, Mass.). Other plasmid constructs and reagents were as described (Su et al., 1998, Proc. Natl. Acad. Sci. U.S.A. 95:14400-14405; Su et al., 2001, Proc. Natl. Acad. Sci. U.S.A. 98:10332-10337; Dent et al., 1999, Mol. Biol. Cell. 10:2493-2506; Hagan et al., 2000, Radiat. Res. 153:371-383; Yacoub et al., 2001, J. Radiat. Biol. 77:1067-1078; Lebedeva et al., 2002, Oncogene 21:708-718)

Generation of Ad.mda-7 and Synthesis of GST-MDA-7. Recombinant type 5 adenovirus to express MDA-7 (Ad.mda-7), control (CMV vector) or control (β-galactosidase) were generated using recombination in HEK293 cells as described (Hitt et al., 1997; Dent et al., 1999, Mol. Biol. Cell. 10:2493-2506). Standard cloning procedures were used to generate a bacterial expression vector comprising in-frame fusion of the mda-7 ORF 3′ to the GST ORF in GST-4T2 vector (Amersham Pharmacia), using BamHI and NotI sites introduced into mda-7 by PCR (Su et al., 2003, Oncogene 22:1164-1180). Expression of protein was performed by inoculating an overnight culture at 1:100 dilution followed by incubation at 25° C. until an O.D.₆₀₀ of 0.4-0.6 was reached followed by induction with 0.1 μM IPTG for 2 h. Cells were harvested by centrifugation and sonicated in PBS followed by centrifugation to obtain soluble protein. The lysate was bound to a glutathione-agarose column (Amersham Pharmacia) at 4° C. for 2 h followed by washing with 50 volumes PBS and 10 volumes PBS with 500 nM NaCl. Elution of bound protein was performed by passing 20 mM reduced glutathione through the column and collecting 1 ml fractions. Fractions were analyzed by gel electrophoresis and positive samples were dialyzed against 1000 volumes of PBS for 4 h with one change followed by 500 volumes of DMEM for 4 h. Protein concentration was estimated by Bradford assays as well as gel electrophoresis in conjunction with Coomassie blue staining. Samples were tested for activity using GST protein as control. Using gel-purified GST-MDA-7, a polyclonal anti-GST-MDA-7 antibody was raised in rabbits and used at a 1:3000 dilution for immunoblotting.

Cell Culture. Fischer rat RT2 gliomablastoma cells (University of Alabama, Birmingham) and primary rodent astrocytes (kindly provided by Dr. E. Ellis, Dept. Pharmacology, V.C.U.) were cultured in DMEM supplemented with 10% fetal bovine serum (Hyclone, Logan, Utah) and 1% penicillin/streptomycin. U251 and U373 human astrocytoma cells were grown in MEM supplemented with 10% (v/v) fetal bovine serum (1×) Nonessential Amino Acids (NEAA), (1×) Sodium Pyruvate. Cells were incubated in humidified atmosphere of 5% CO₂ at 37° C.

Recombinant Adenovirus Infection. The Ad.mda-7 and control adenoviral vectors used were identical to those described previously (Su et al., 1998, Proc. Natl. Acad. Sci. U.S.A. 95:14400-14405; Su et al., 2001, Proc. Natl. Acad. Sci. U.S.A. 98:10332-10337; Lebedeva et al., 2002, Oncogene 21:708-718). The viral titers for each adenovirus and infection efficiency for each cell type were determined by plaque formation assay. In vitro adenoviral infections and β-galactosidase/X-gal staining were performed 24 h after plating (Volkert and Young, 1983, Virology 125:175-193). Monolayer cultures were washed in PBS and incubated with purified virus in 1 ml of growth medium without serum for 1 h at 37° C. in a humidified atmosphere of 5% CO₂/95% air with gentle agitation. After 3 h, fresh growth medium with 10% fetal bovine serum was added.

Assessment of Apoptosis and Cell Viability. The extent of apoptosis was evaluated by assessing Wright-Giemsa stained cytospin slides under light microscopy and scoring the number of cells exhibiting the classic morphological features of apoptosis. For each condition, 10 randomly selected fields per slide were evaluated, encompassing at least 15000 cells (Grant et al., 1996). To confirm the results of morphologic analysis, in some cases cells were also evaluated by TUNEL staining and oligonucleosomal DNA fragmentation assay as follows; staining, cytospin slides were fixed with 4% formaldehyde/PBS for 10 min, treated with acetic acid/ethanol (1:2) for 5 min, and incubated with terminal transferase reaction mixture containing IX terminal transferase reaction buffer, 0.25 U/l terminal transferase, 2.5 mM CoCl₂, and 2 pmol fluorescein-12-dUTP (Boehringer Mannheim, Indianapolis, Ind.) at 37° C. for 1 h. The slides were mounted with Vectashield containing propidium iodide (Vector Laboratories, Burlingame, Calif.) and visualized using fluorescence microscopy (Dai et al., 2002, Cell Cycle 1: 143-152).

Assessment of Cell Viability. Cell viability was also evaluated by assessing trypan blue inclusion/exclusion of isolated cells under light microscopy and scoring the percentage of cells exhibiting blue staining (Cartee et al., 2000, Int. J. Oncol. 16:413-422). Floating and attached cells were isolated by trypsinization, recovered by centrifugation, resuspended in phenol red free DMEM and mixed 1:1 with trypan blue reagent. Cells (˜400) were counted in all four fields of a hemocytometer.

MIT Assay for Determination of Cellular Viability. The MTT test is based on the enzymatic reduction of the tetrazolium salt MTT in living, metabolically active cells. Cells were plated (5-10,000 cells per well of a 12 well plate) and 24 h after plating infected with either Ad.mda-7 or control virus at the indicated multiplicity of infection (m.o.i.). In other experiments, cells were plated (5-10,000 cells per well of a 12 well plate) and 24 h after plating treated with either GST or GST-MDA-7 at the indicated concentrations. Twenty-four hours after infection/protein treatment, cells were treated with kinase inhibitor drugs and then irradiated. The cytotoxicity of the various treatments was assessed four days after irradiation by measurement of cell viability by use of the MTT assay, as described previously (Mosmann, 1983, J. Immunol. Methods 65:55-63). The plates were read on a Dynatech MR600 Microplate Reader at 540 nm. All data were normalized relative to the control, non-treated unirradiated cells of the corresponding cell type.

Cell Survival Analyses. Cells were assayed for the effect(s) of Ad.mda-7, and radiation on cell survival. Cells were plated (10,000 cells per 60 mm dish) and 24 h after plating infected with either Ad.mda-7 or control virus at the indicated m.o.i. Twenty four hours after infection cells were irradiated. Ninety-six hours after irradiation, cells were isolated by trypsinization and viable trypan blue negative cells re-plated in 60 mm dishes at 250-1,000 cells per plate. Colonies were allowed to from surviving cells for 7-9 days, before fixing and staining with crystal violet. Colonies that contain more than 50 cells were then counted. To generate the survival data, individual assays were performed at multiple dilutions with a total of four plates per data point.

Cell Cycle Analysis. The cells were treated as described for the MTT assay above (150000 cells/60 mm dish). At the time of irradiation and 24 h after irradiation, cells were isolated to determine their cell cycle profiles. Cells were washed once with PBS, fixed in 80% ice-cold ethanol, centrifuged, washed with phosphate-buffered saline (PBS), and ˜10⁶ cells per condition were stained with propidium iodide (50 μg/mL) in PBS containing 100 μg/mL RNase A. The cells were subjected to flow cytometric analysis of DNA content using a Becton Dickinson FACScalibur cytometer and analyzed using Verity Winlist software.

Western Blot Analysis. Protein concentration was determined using a kit from Bio-Rad. Aliquots (40 μg) were solubilized in Laemmli buffer, separated by SDS-PAGE, and transferred to nitrocellulose membranes as described. Membranes were blocked for 2 hours at 4° C. in TBST (5% nonfat milk in 10 mM Tris/HCl, 100 nM NaCl, and 0.1% Tween-20, pH 7.6). Membranes were exposed to the primary antibodies, followed by washing (3×15 min with TBST). The following antibodies were used: mouse anti-PARP, anti-p21 and anti-beta-actin (Santa Cruz Biotechnology, CA); anti-p53 (polyclonal antibody (Oncogene Research Products, Cambridge, Mass.); ERK1/2, JNK1/2, P38 and AKT phosphorylation was determined by using phosphospecific antibodies (Cell Signaling, Beverly, Mass.) (Dai et al., 2002, Cell Cycle 1:143-152; Qiao et al., 2002, Hepatology 36:39-48). Total ERK antibody (Santa Cruz) was used as a loading control. Membranes were incubated with horseradish peroxidase-conjugated anti-mouse or anti-rabbit IgG antibody, followed by washing with TBST (3×15 min). Proteins were visualized by enhanced chemiluminescence and quantified by densitometry.

DNA Fragmentation. Equal numbers of cells from each test sample (10⁶) were homogenized with 1 ml lysis buffer (10 mM Tris at pH 7.4, 5 mM EDTA, 1% Triton X-100). RNase A 100 μg/ml was added to each sample and incubated at 50° C. for 1 hour. Proteinase K was then added (100 μg/ml) and the samples were incubated overnight for at 50° C. The DNA was extracted using phenol and chloroform, and centrifuged at 10,000×g for 5 min at 4° C. The aqueous phase mixed with 2 volumes of ice-cold ethanol and then precipitate by centrifugation at 15,000×g for 10 min, supernatants were removed, and DNA pellets were washed with 80% ethanol once (15,000 times; g for 10 min), air-dried, dissolved in TE buffer at pH 7.6. DNA concentration were determined and 10 μg of each sample was then electrophoresed on a 1.5% agarose gel and analyzed for the presence of a laddering pattern.

Statistical Analyses. Comparison of the effects of various treatments was performed using one way analysis of variance and a two tailed t-test. Differences with a p-value of <0.05 were considered statistically significant. Experiments shown are the means of multiple individual points±SEM).

Results

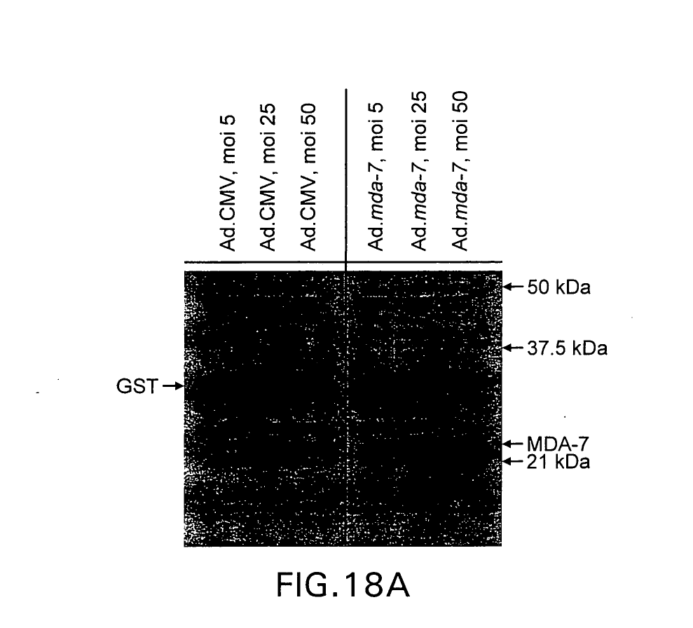

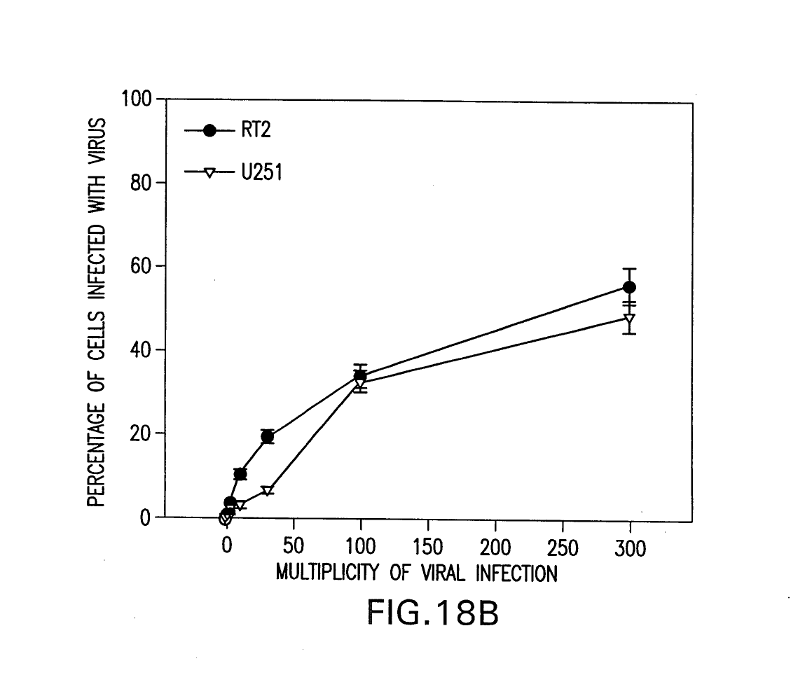

Ad.mda-7 infected RT2 cells express a 23 kDa MDA-7 protein. RT2 cells were infected with increasing amounts of Ad.mda-7 or control virus and the expression of MDA-7 determined 48 h after infection in cell lysates (FIG. 18A). Increasing the viral particle multiplicity of infection (m.o.i.) enhanced the amount of MDA-7 protein produced in each cell. Similar data were obtained in U251 astrocytoma cells, in agreement the previous study of Su et al. (Su et al., 2003 Oncogene 22:1164-1180).

In parallel with experiments examining MDA-7 expression, additional studies using a recombinant adenovirus to express β-galactosidase were performed to determine the percentage of cells infected at each m.o.i. (FIG. 18B). Infection at an m.o.i. of 25 with a virus to express β-galactosidase caused approximately 20% of RT2 and 5% of U251 cells to stain blue when incubated with X-gal.

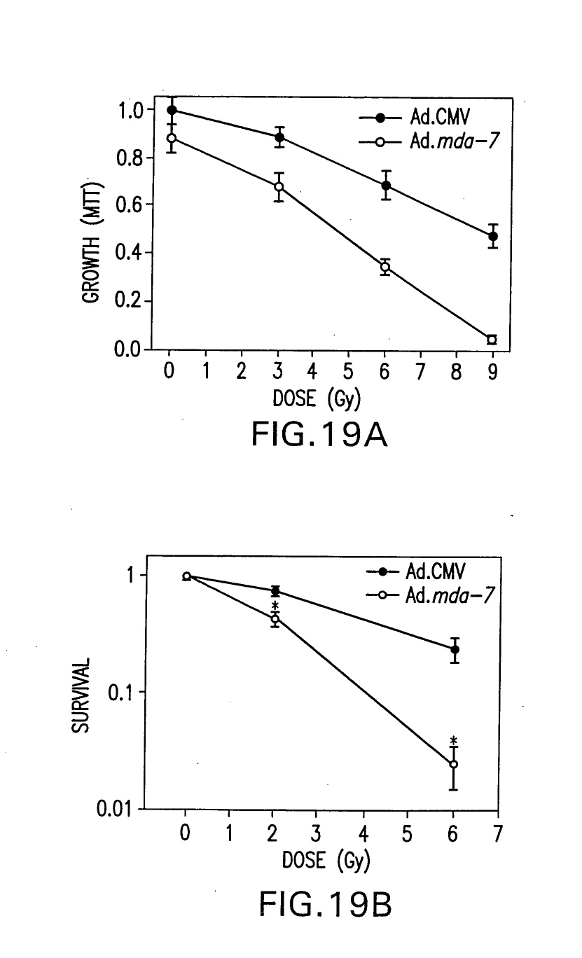

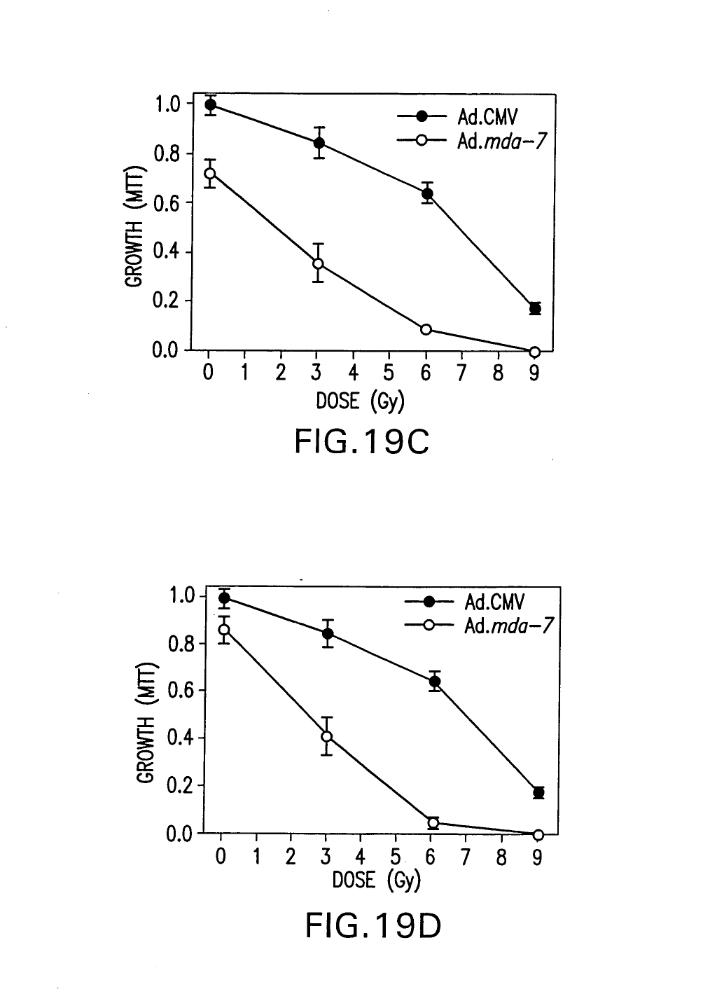

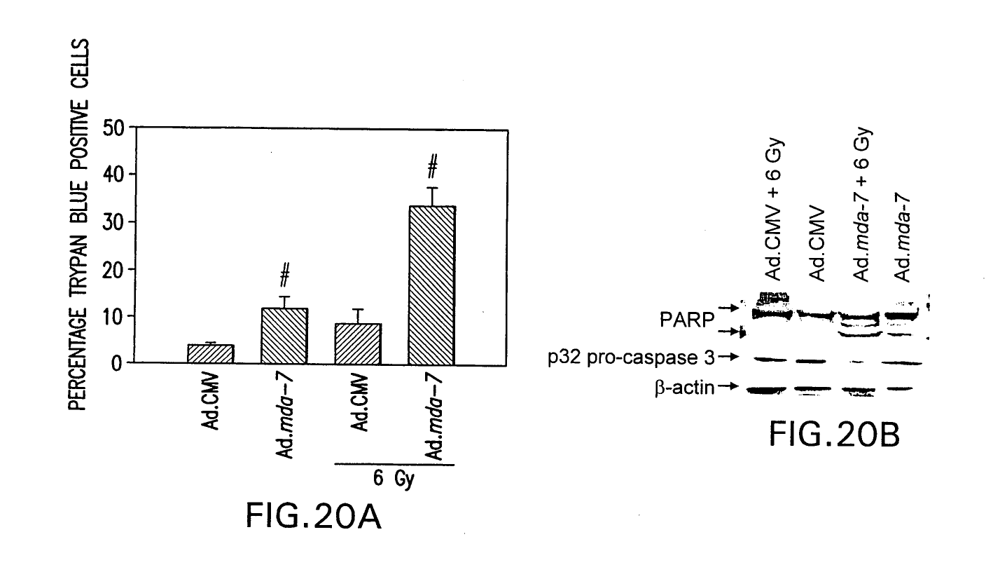

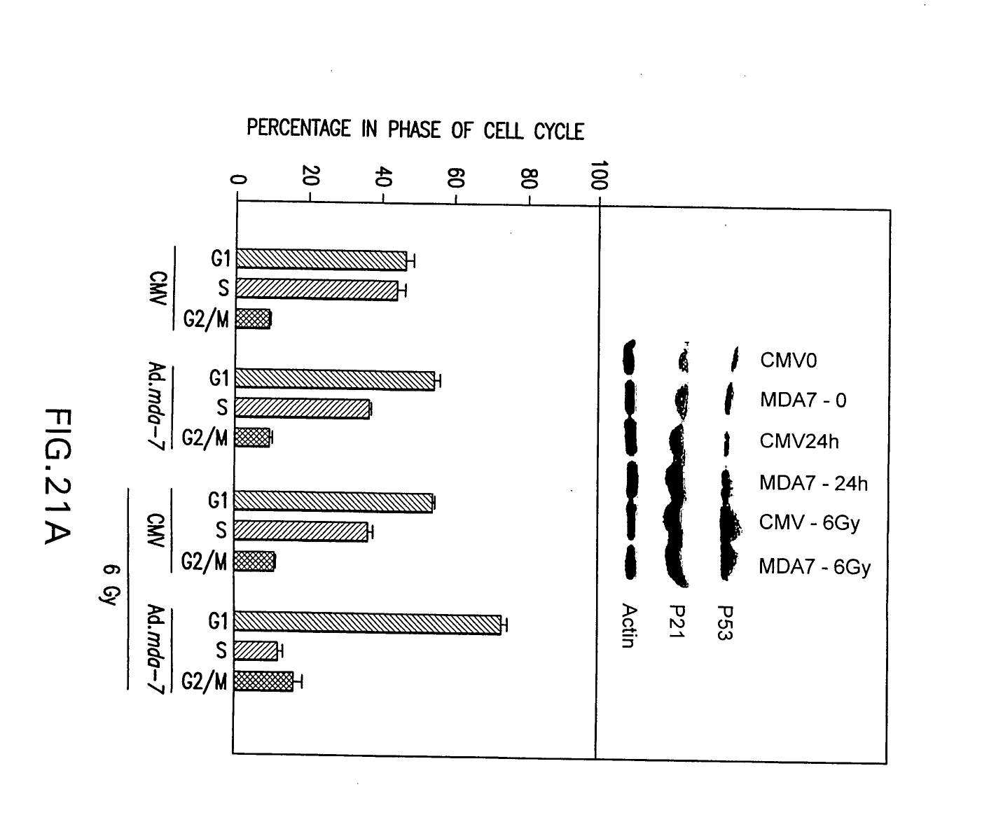

Ad.mda-7 enhances the radiosensitivity of RT2, U251 and U373 cells. Previous studies have shown that infection of tumor cells, but not non-transformed cells, with Ad.mda-7 inhibited tumor cell growth (Su et al., 1998, Proc. Natl. Acad. Sci. U.S.A. 95:14400-14405; Madireddi et al., 2000, Adv. Exp. Med. Biol. 465:239-261; Saeki et al., 2000, Gene Ther. 7:2051-2057; Mhashilkar et al., 2001, Mol. Med. 7:271-282; Su et al., 2001, Proc. Natl. Acad. Sci. U.S.A. 98:10332-10337; Sarkar et al., 2002, Biotechniques October:30-39; Sarkar et al., 2002, Proc. Natl. Acad. Sci. U.S.A. 99:10054-10059; Saeki et al., 2002, Oncogene 21:4558-4566; Ellerhorst et al., 2002, J. Clin. Oncol. 20:1069-1074; Lebedeva et al., 2002, Oncogene 21:708-718; Sauane et al., 2003, J. Cell. Physiol. 196:334-345; Su et al., 2003 Oncogene 22:1164-1180) To assess the effect of Ad.mda-7 on the growth and survival of RT2, U251 and U373 glioma cells, cells were assayed for proliferation via MTT assay, survival via colony formation and cell viability via trypan blue staining after Ad.mda-7 infection (25 m.o.i.) and radiation exposure. Cells were plated, infected and irradiated 24 h after infection with increasing radiation doses (FIGS. 19A-19D; FIGS. 20A-20C). Radiation reduced proliferation and it interacted with Ad.mda-7, but not control virus, to further reduce cell growth. These effects were not observed in either primary rodent or human astrocytes (Su et al., 2003, Oncogene 22:1164-1180).

In general agreement with the findings in FIG. 19, Ad.mda-7-enhanced cell death, as judged by trypan blue staining that was significantly increased following radiation exposure (FIG. 20A). This effect was not observed in primary rodent astrocytes. As an additional separate measure of cell survival, the integrity of poly ADP ribosyl polymerase (PARP), p32 pro-caspase 3 and nuclear DNA, 96 h after irradiation/20 h after viral infection was determined. Irradiation and/or infection of cells with control virus did not significantly alter PARP, pro-caspase 3 and DNA integrity (FIG. 20B). However, infection of cells with Ad.mda-7 caused cleavage of PARP and pro-caspase 3 and the appearance of a “smear” of lower molecular weight DNA, suggestive of necrosis.

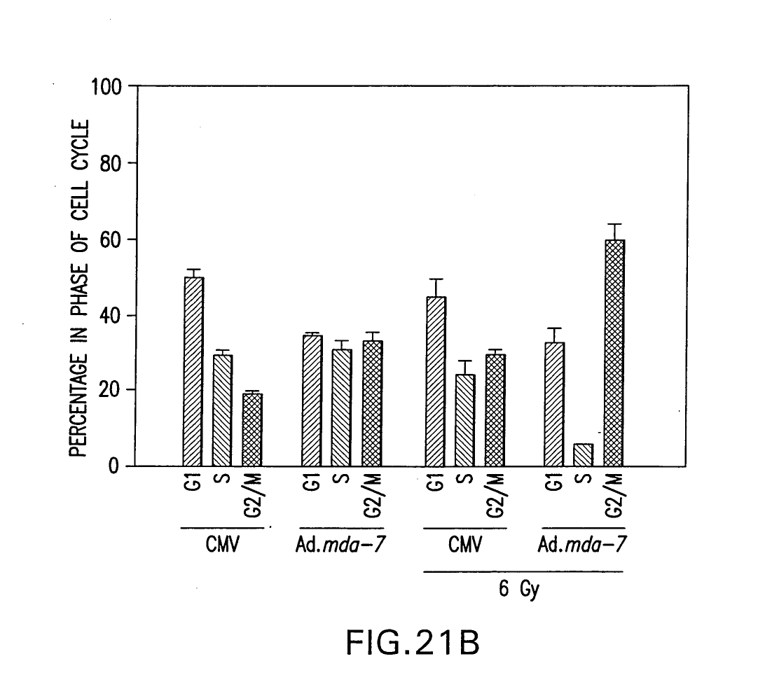

The reduction in proliferation caused by Ad.mda-7 was further examined in RT2 and U251 cells. In RT2 cells, Ad.mda-7 enhanced cell numbers in G₁ phase that was further increased after irradiation (FIG. 21A). This correlated with enhanced expression of p21 and p53 (inset panel). In contrast, in U251 cells, Ad.mda-7 enhanced cell numbers in G₂/M phase of the cell cycle that was further increased following radiation exposure (FIG. 21B); expression of p53 and p21 was unaltered in U251 cells. Cell numbers in S phase significantly declined in both cell types following combined Ad.mda-7 and radiation treatment.

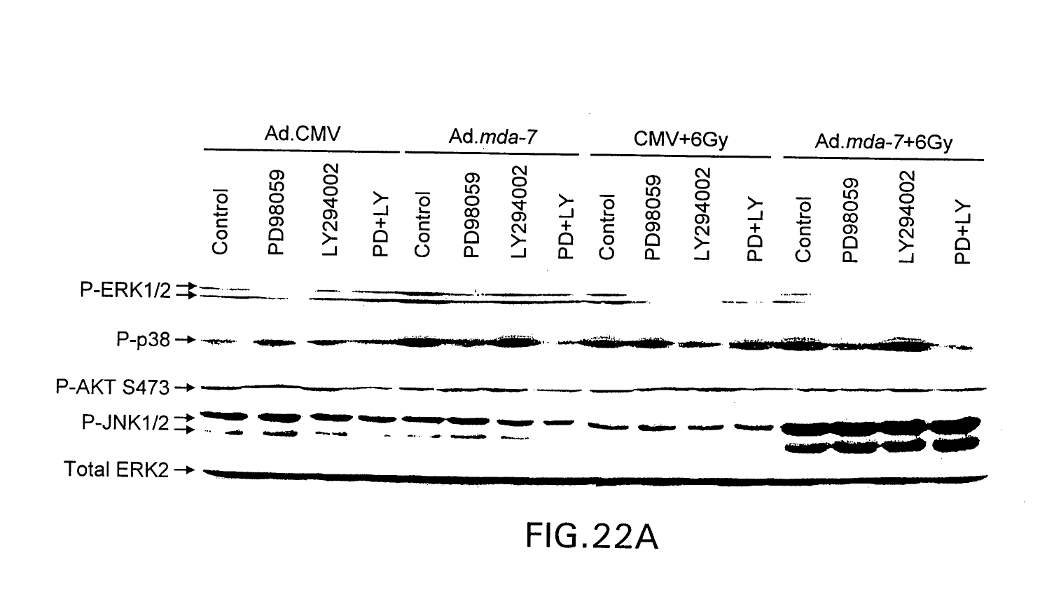

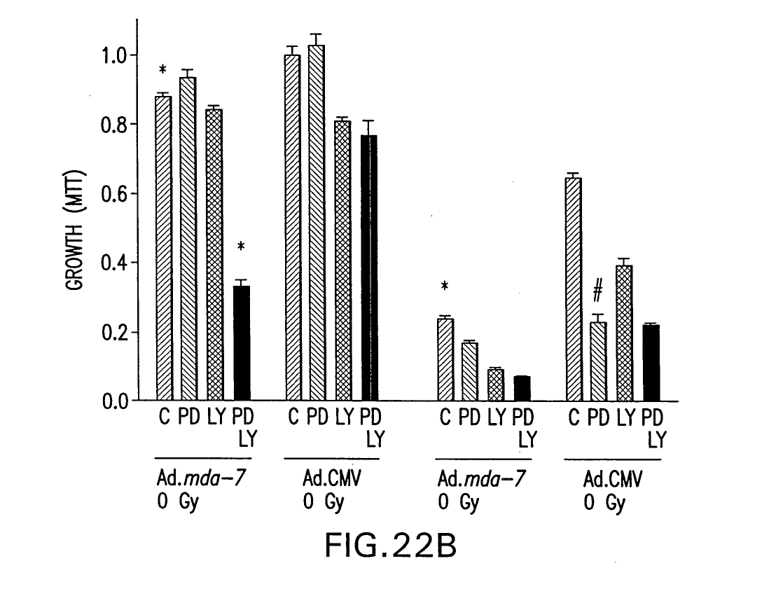

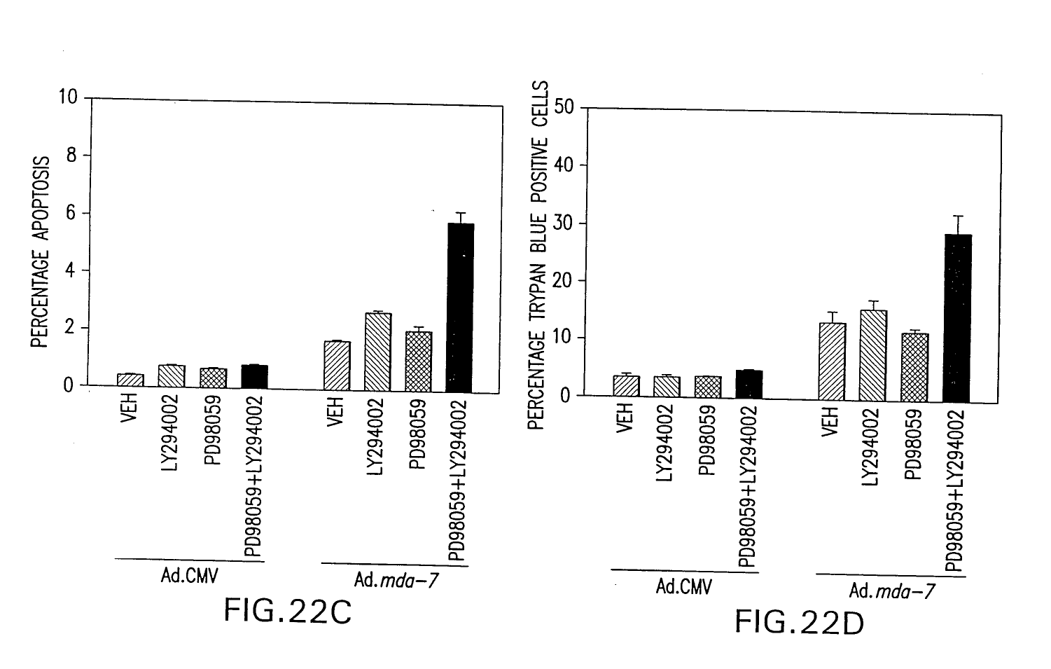

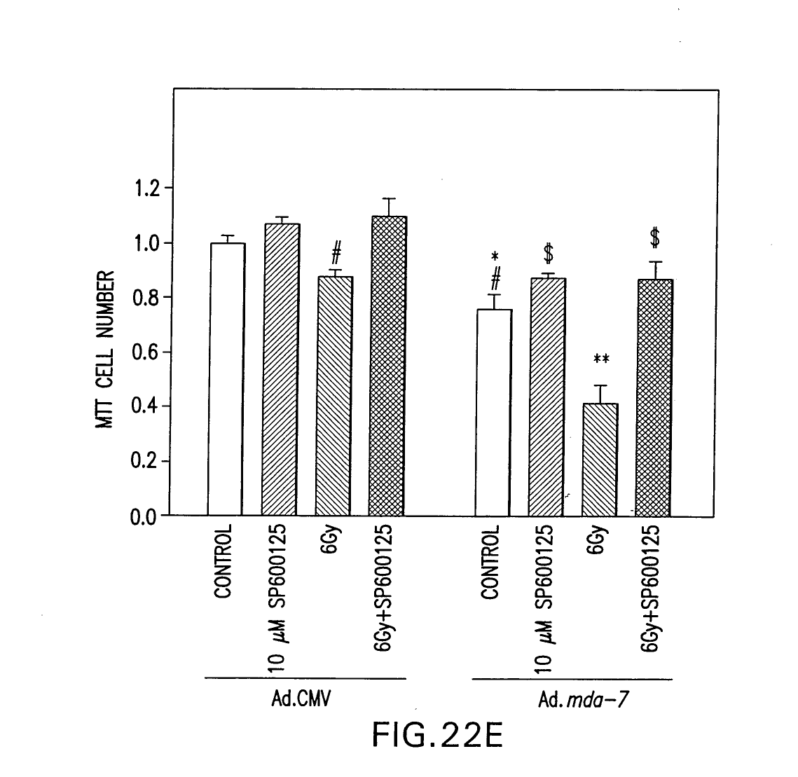

Ad.mda-7 enhances the activity of ERK1/2 and P38, but not JNK and AKT, in RT2 cells. Recent studies have linked MDA-7-induced cell killing to activation of the P38 and JNK pathways (Kawabe et al., 2002, Mol. Ther. 6:637-644; Sarkar et al., 2002, Proc. Natl. Acad. Sci. U.S.A. 99:10054-10059). Signaling by mutant RAS has also been shown to enhance resistance to both radiation and Ad.mda-7 (Gupta et al., 2001, Cancer Res. 61; Su et al., 2001, Proc. Natl. Acad. Sci. U.S.A. 98:10332-10337). JNK signaling has also been proposed as the mechanism by which Ad.mda-7 radiosensitizes lung carcinoma cells (Kawabe et al., 2002, Mol. Ther. 6:637-644). Based on these findings, further studies investigated whether Ad.mda-7 and radiation interacted to alter the activities of the ERK, JNK, P38 and AKT in RT2 cells. Infection with Ad.mda-7 enhanced the activity of P38 and ERK1/2, but not of JNK1/2 or AKT (FIG. 22A). Surprisingly, radiation reduced Ad.mda-7-induced ERK1/2 activity 96 h after exposure, but had no effect on either P38 or AKT activity. Of note, however, while Ad.mda-7 did not alter basal JNK1/2 activity, following irradiation Ad.mda-7 considerably enhanced JNK1/2 phosphorylation.

Signaling by the PI3K/AKT and ERK/MAPK pathways have been linked to enhanced radioresistance and survival to chemotherapy (Vlahos et al., 1994, J. Biol. Chem. 269:5241-5248; Xia et al., 1995, Science 270:1326-1331; Dent et al., 1999, Mol. Biol. Cell. 10:2493-2506; Cartee et al., 2000, Int. J. Oncol. 16:413-422; Hagan et al., 2000, Radiat. Res. 153:371-383; Gupta et al., 2001, Cancer Res. 61:4278-4282; Gupta et al., 2002, Clin. Cancer Res. 8:885-892; Yacoub et al., 2001, J. Radiat. Biol. 77:1067-1078; Sarkar et al., 2002, Proc. Natl. Acad. Sci. U.S.A. 99:10054-10059). To investigate whether these pathways play a role in resistance to MDA-7-induced cell killing, cells were infected with Ad.mda-7 and then incubated with the MEK1/2 inhibitor PD98059 and the PI3K inhibitor LY294002. Inhibition of neither MEK1/2 nor PI3K enhanced the anti-proliferative effects of Ad.mda-7 (FIG. 22B). However, combined inhibition of both pathways caused a significant additional reduction in proliferation (FIG. 22B) and cell survival (FIGS. 22C and 22D).

Because infection of cells with Ad.mda-7 activated p38 as well as promoting radiation-induced activation of JNK in RT2 cells, an investigation of whether inhibition of either p38 or JNK signaling was involved in the radiosensitization effect was performed. Based on previous studies, it was expected p38 signaling to play an important role in enhanced cell killing by Ad.mda-7 and radiation (Sarkar et al., 2002, Proc. Natl. Acad. Sci. U.S.A. 99:10054-10059), however inhibition of p38 signaling did not alter the radiosensitizing properties of Ad.mda-7. In contrast, and in general agreement with the findings of Kawabe et al. (Kawabe et al., 2002, Mol. Ther. 6:637-644), use of a relatively specific JNK1/2 inhibitor SP600125, abolished the radiosensitizing properties of Ad.mda-7 (FIG. 22E).

### 6.4. Example 4

### Free Radical Generators in Combination with mda-7 Nucleic Acid or MDA-7 Protein as a Treatment for Renal Cell Carcinoma

Materials and Methods

Reagents. Dulbecco's Modified Eagle's Medium (DMEM) and Penicillin-Streptomycin were from Gibco (Life Technology, New York). Nonessential Amino Acids (NEAA), and Sodium Pyruvate were from Cellgro (VA). MTT reagent (3-[4,5-Dimethylthiazol-2-yl]-2,5-diphenyltetrazolium bromide), and Giemsa Stain were from Sigma (St. Louis, Mo.). Anti-Caspase 3, Phospho-/total-ERK1/2, Phospho-/total-P38α/β, Phospho-/total-JNK1/2, Anti-Bcl-2, Anti-Bcl-x_{L}, Anti-FAS receptor, Anti-FAS ligand, Anti-Bax and all the secondary antibodies (anti-rabbit-HRP, anti-mouse-HRP, and anti-goat-HRP) were purchased from Santa Cruz Biotechnology (Santa Cruz, Calif.). Anti-PARP (1:2500, mouse monoclonal) was obtained from Calbiochem. Enhanced chemiluminescence (ECL) kit was purchased from NEN Life Science Products (Boston, Mass.). Other constructs and reagents were as described in (Jiang et al., 1996, Proc. Natl. Acad. Sci. U.S.A. 93:9160-9165; Su et al., 1998, Proc. Natl. Acad. Sci. U.S.A. 95:14400-14405; Madireddi et al., 2000, Adv. Exp. Med. Biol. 465:239-261; Saeki et al., 2000, Gene Ther. 7:2051-2057; Su et al., 2003, Oncogene 22:1164-1180).

Generation of Ad.mda-7 and Synthesis of GST-MDA-7. Recombinant type 5 adenovirus to express MDA-7 (Ad.mda-7), control (CMV vector) or control (β-galactosidase) were generated using recombination in HEK293 cells.

Standard cloning procedures were used to generate a bacterial expression vector comprising in-frame fusion of the mda-7 ORF 3′ to the GST ORF in GST-4T2 vector (Amersham Pharmacia), using BamHI and NotI sites introduced into mda-7 by PCR (Su et al., 2003 Oncogene 22:1164-1180). Expression of protein was performed by inoculating an overnight culture at 1:100 dilution followed by incubation at 25° C. until an O.D.₆₀₀ of 0.4-0.6 was reached followed by induction with 0.1 μM IPTG for 2 h. Cells were harvested by centrifugation and sonicated in PBS followed by centrifugation to obtain soluble protein.

The lysate was bound to a glutathione-agarose column (Amersham Pharmacia) at 4° C. for 2 h followed by washing with 50 volumes PBS and 10 volumes PBS with 500 mM NaCl. Elution of bound protein was performed by passing 20 mM reduced glutathione through the column and collecting 1 ml fractions. Fractions were analyzed by gel electrophoresis and positive samples were dialyzed against 1000 volumes of PBS for 4 h with one change followed by 500 volumes of DMEM for 4 h. Protein concentration was estimated by Bradford assays as well as gel electrophoresis in conjunction with Coomassie blue staining. Samples were tested for activity using GST protein as control. Using gel-purified GST-MDA-7, a polyclonal anti-GST-MDA-7 antibody was raised in rabbits and used at a 1:3000 dilution for immunoblotting.

Cell Culture. Human U373 glioma cells (ATCC) and human RCC cells: A498 (ATCC) UOK121N (Dr. Lineham, National Cancer Institute) and primary human renal epithelial cells (Clonetics, Cambrex Corp., East Rutherford, N.J.) were cultured in DMEM supplemented with 10% fetal bovine serum (Hyclone, Logan, Utah) and 1% penicillin/streptomycin according to the suppliers instructions. Cells were incubated in humidified atmosphere of 5% CO₂ at 37° C.

Primary Culture of Rodent Hepatocytes. Hepatocytes were isolated from adult male Sprague Dawley rats by the two-step collagenase perfusion technique (Gupta et al., 2001, J. Biol. Chem. 276:15816-15822; Qiao et al., 2001, Mol. Biol. Cell 12:2629-2645). The freshly isolated hepatocytes were plated on rat-tail collagen (Vitrogen)-coated at a density of 2.5×10⁵ cells/well, and cultured in DMEM supplemented with 250 nM insulin, 0.1 nM dexamethasone, 1 nM thyroxine, and 100 μg/ml of penicillin/streptomycin, at 37° C. in a humidified atmosphere containing 5% CO₂. An initial medium change was performed 4 hr after cell seeding, at the time of viral infection, to remove dead or mechanically damaged cells.

Recombinant Adenovirus Infection. The Ad.mda-7 and control adenoviral vectors used were identical to those described previously (Madireddi et al., 2000, Adv. Exp. Med. Biol. 465:239-261; Saeki et al., 2000, Gene Ther. 7:2051-2057; Su et al., 1998, Proc. Natl. Acad. Sci. U.S.A. 95:14400-14405; Su et al., 2001, Proc. Natl. Acad. Sci. U.S.A. 98:10332-10337, Su et al., 2003 Oncogene 22:1164-1180; Mhashilkar et al., 2001, Mol. Med. 7:271-282; Lebedeva et al., 2002, Oncogene 21:708-718). The viral titers for each virus and infection efficiency for each cell type were determined by plaque formation assay. In vitro adenoviral infections were performed 24 h (RCC) and 4 h (hepatocytes) after plating. Monolayer cultures were washed in PBS and incubated with purified virus in 1 ml of growth medium without serum for 1 h at 37° C. in a humidified atmosphere of 5% CO₂/95% air with gentle agitation. For RCC lines, after 3 h, fresh growth medium with 10% fetal bovine serum was added.

Assessment of Apoptosis and Cell Death. The extent of apoptosis and necrosis (cell death) was evaluated by assessing Wright-Giemsa stained cytospin slides under light microscopy and scoring the number of cells exhibiting the classic morphological features of apoptosis. For each condition, 10 randomly selected fields per slide were evaluated, encompassing at least 15000 cells (Dai et al., 2002, Cell Cycle 1:143-152; Qiao et al., 2001, Mol. Biol. Cell 12:2629-2645). To confirm the results of morphologic analysis, in some cases cells were also evaluated by TUNEL staining and oligonucleosomal DNA fragmentation assay as follows; staining, cytospin slides were fixed with 4% formaldehyde/PBS for 10 min, treated with acetic acid/ethanol (1:2) for 5 min, and incubated with terminal transferase reaction mixture containing 1× terminal transferase reaction buffer, 0.25 U/l terminal transferase, 2.5 mM CoCl₂, and 2 pmol fluorescein-12-dUTP (Boehringer Mannheim, Indianapolis, Ind.) at 37° C. for 1 h. The slides were mounted with Vectashield containing propidium iodide (Vector Laboratories, Burlingame, Calif.) and visualized using fluorescence microscopy.

Assessment of Cell Viability. Cell viability was also evaluated by assessing trypan blue inclusion/exclusion of isolated cells under light microscopy and scoring the percentage of cells exhibiting blue staining (Yu et al., 2001, Biochem. Biophys. Res. Commun. 286:1011-1018). Floating and attached cells were isolated by trypsinization, recovered by centrifugation, resuspended in phenol red free DMEM and mixed 1:1 with trypan blue reagent. Cells (˜400) were counted in all four fields of a hemocytometer.

MTT Assay for Determination of Cellular Viability. The MTT test is based on the enzymatic reduction of the tetrazolium salt MTT in living, metabolically active cells but not in dead cells. Cells were plated (5-10,000 cells per well of a 12 well plate) and 24 h after plating treated with either GST or GST-MDA-7 at the indicated concentrations. Thirty minutes after protein treatment, cells were treated with arsenic trioxide at the indicated concentrations. The cytotoxicity of the various treatments was assessed four days after irradiation by measurement of cell viability by use of the MTT assay, as described previously (McKinstry et al., 2002, Cancer Biol. Ther. 1:243-253). The plates were read on a Dynatech MR600 Microplate Reader at 540 nm. All data were normalized relative to the GST control, non-treated cells of the corresponding cell type.

Cell Survival Analyses. Cells were assayed for the effect(s) of GST-MDA-7 and arsenic trioxide on cell survival. Cells were plated (10,000 cells per 60 mm dish) and 24 h after plating treated with GST-MDA-7 or GST. Thirty minutes later, cells were treated with arsenic trioxide. Ninety-six hours later, cells were isolated by trypsinization and viable trypan blue negative cells re-plated in 60 mm dishes at 250-1,000 cells per plate. Colonies were allowed to from surviving cells for 10-14 days, before fixing and staining with crystal violet (Yu et al., 2001, Biochem. Biophys. Res. Commun. 286:1011-1018; McKinstry et al., 2002, Cancer Biol. Ther. 1:243-253). Colonies that contain more than 50 cells were then counted. To generate the survival data, individual assays were performed at multiple dilutions with a total of six plates per data point repeated for a total of three experiments.

Western Blot analysis. Protein concentration was determined using a kit from Bio-Rad. Aliquots (40 μg) were solubilized in Laemmli buffer, separated by SDS-PAGE, and transferred to nitrocellulose membranes. Membranes were blocked 2 hours at 4° C. in TBST (5% nonfat milk in 10 mM Tris-HCl, 100 mM NaCl, and 0.1% Tween-20, pH 7.6). Membranes were exposed to the primary antibodies, followed by washing (3×15 min with TBST). Membranes were incubated with horseradish peroxidase-conjugated anti-mouse or anti-rabbit IgG antibody, followed by washing with TBST (3×15 min). Proteins were visualized by enhanced chemiluminescence.

DNA Fragmentation. Equal number of cells from each test (10⁶) homogenized with 1 ml lysis buffer (10 mM Tris at pH 7.4, 5 mM EDTA, 1% Triton X-100). RNase A 100 μg/ml was added to each sample and incubated at 50° C. for 1 hour. Proteinase K was then added (100 μg/ml) and the samples were incubated overnight for at 50° C. The DNA was extracted using phenol and chloroform, and centrifuged at 10,000×g for 5 min at 4° C. The aqueous phase mixed with 2 volumes of ice-cold ethanol and then precipitate by centrifugation at 15,000×g for 10 min, supernatants were removed, and DNA pellets were washed with 80% ethanol once (15,000×g for 10 min), air-dried, dissolved in TE buffer at pH 7.6. DNA concentrations were determined and 10 μg of each sample was then electrophoresed on a 1.5% agarose gel and analyzed for the presence of a laddering pattern.

Statistical Analyses. Comparison of the effects of various treatments was performed using one way analysis of variance and a two tailed t-test. Differences with a p-value of <0.05 were considered statistically significant. Experiments shown are the means of multiple individual points (±SEM).

Results

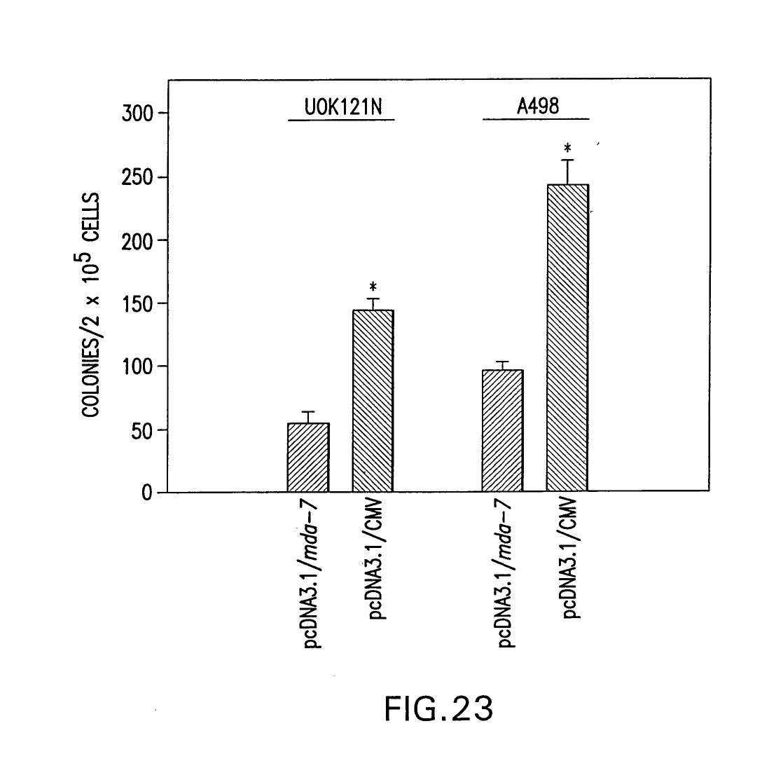

The RCC cell lines A498 and UOK121N are weakly responsive or refractory to Ad.mda-7 treatment but strongly responsive to transfection by an mda-7-expressing plasmid. Previous studies performed by the inventors have shown that tumor cells, but not non-transformed cells, infected with the type 5 recombinant adenovirus, Ad.mda-7 undergo growth arrest and apoptosis (Su et al., 1998, Proc. Natl. Acad. Sci. U.S.A. 95:14400-14405; Mhashilkar et al., 2001, Mol. Med. 7:271-282; Su et al., 2001, Proc. Natl. Acad. Sci. U.S.A. 98:10332-10337; Lebedeva et al., 2002, Oncogene 21:708-718; Sarkar et al., 2002, Proc. Natl. Acad. Sci. U.S.A. 99:10054-10059; Sauane et al., 2003, J. Cell. Physiol. 196:334-345; Su et al., 2003 Oncogene 22:1164-1180). Initial studies in renal cell carcinoma (RCC) lines attempted to recapitulate these prior findings using Ad.mda-7 in other tumor cell types. However, the inventors were unable to observe any effect of Ad.mda-7 on A498 cell growth and observed only a weak effect on the growth of UOK121N cells. In contrast, transfection of RCC lines with a plasmid to express MDA-7 reduced colony formation and cell growth (FIG. 23) (Jiang et al., 1996, Proc. Natl. Acad. Sci. U.S.A. 93:9160-9165; Su et al., 2001, Proc. Natl. Acad. Sci. U.S.A. 98:10332-10337).

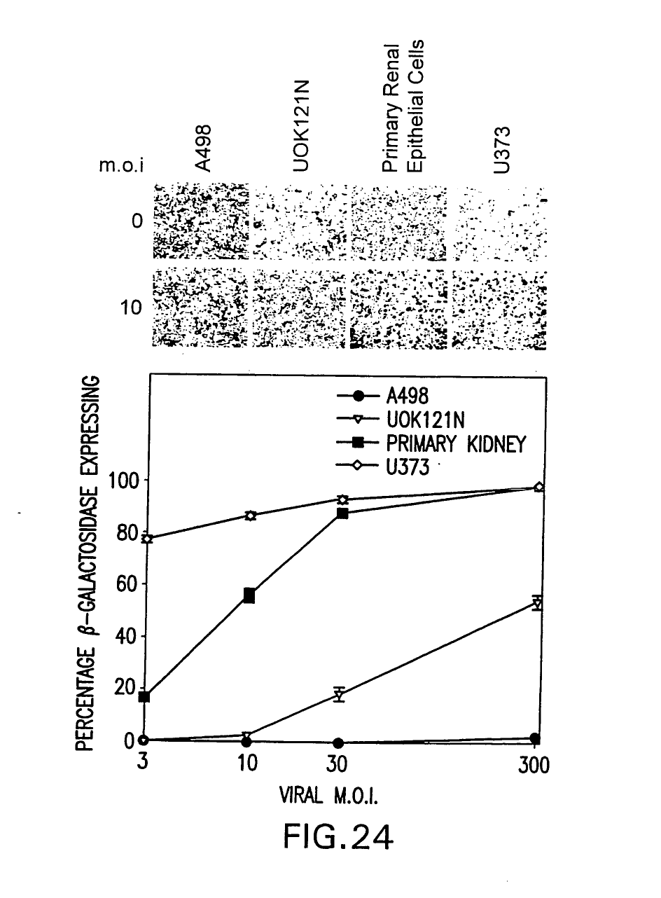

The RCC cell lines A498 and UOK121N express low levels of the Coxsackie-Adenovirus Receptor (CAR) protein. Since transfection, but not infection, of RCC lines to express MDA-7 resulted in reduced cell growth, the inventors determined whether RCC lines express the coxsackievirus/adenovirus receptor (CAR), which is necessary for adenovirus entry into cells. In the two RCC lines tested, CAR levels were very low, in contrast to either U373 malignant glioma cells or primary human renal epithelial cells (Table 4). This is similar to the findings of Haviv et al. (Haviv et al., Cancer Res. 62:4273-4281). Furthermore, when cells were exposed to increasing doses of an adenovirus to express β-galactosidase, it was noted that the virus more easily infected U373 and primary renal epithelial cells than A498 or UOK121N RCC lines (FIG. 24).

TABLE 4
Primary renal epithelial cells, but not renal cell carcinoma cells
express CAR proteins. Cells were cultured as described in the Materials
and Methods section of this example. Forty eight hours after plating,
cells were isolated and incubated with anti-CAR or control antibodies.
Cells were incubated with an FITC labeled secondary antibody and
subjected to flow cytometry to determine CAR levels. Cells incubated
with only control primary antibody or with only secondary FITC labeled
antibody did not display any cell labeling. The “Peak shift” is
calculated as a ratio (P_{CAR} − P_{control})/P _{control}, where P is
median of fluorescent peak of FACS histogram. The D value represents
the statistical difference between the two FACS histogram curves
(Kolmogorov-Smimov test; *p < 0.05 less than primary renal epithelial
cells); Higher numbers reflect enhanced binding and increased CAR
vailability. A representative experiment is shown (n = 2).

| | U373 | A498 | UOK121N | Primary |
|---|---|---|---|---|
| Peak Shift | 6.43 | 0.11 | 0.11 | 9.1 |
| D value | 0.85 | 0.01* | 0.09* | 0.86 |

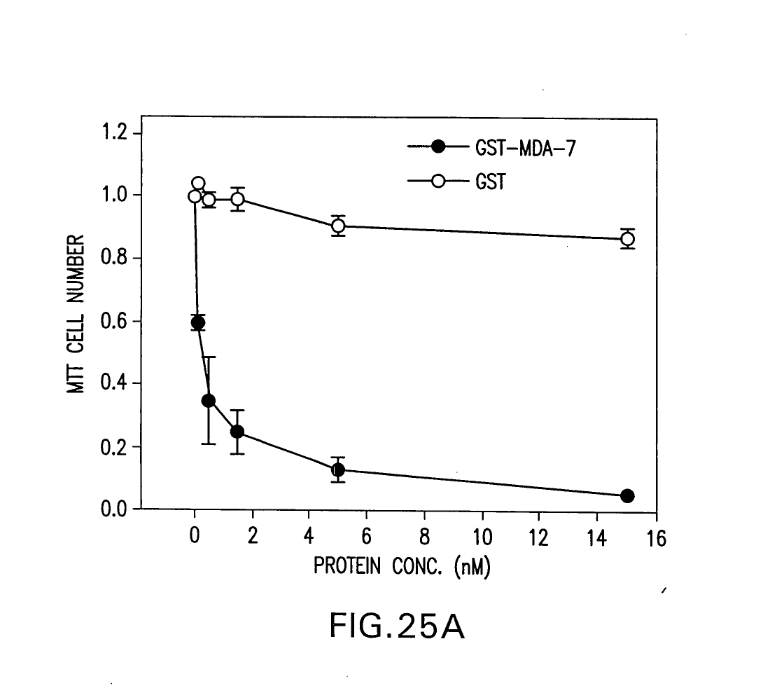

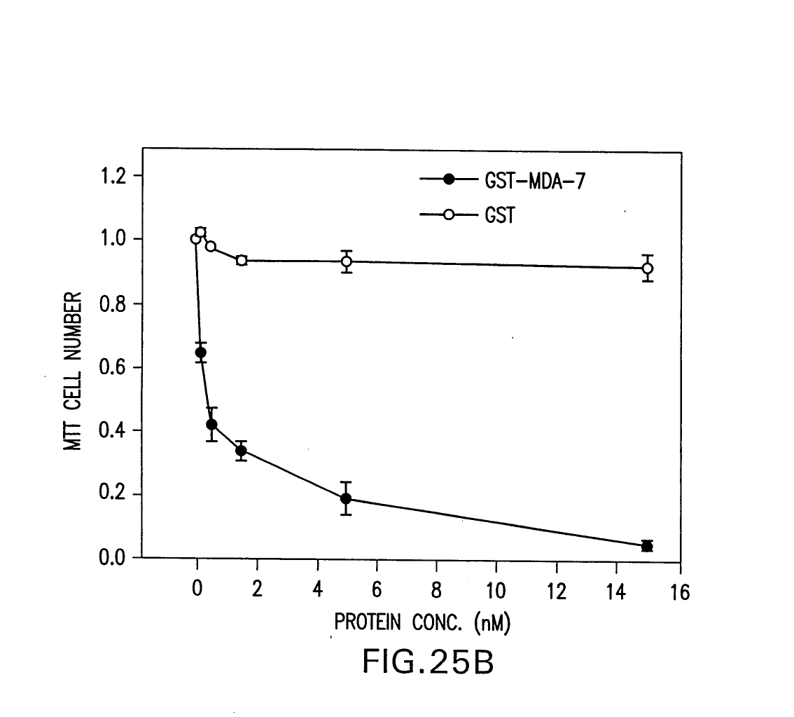

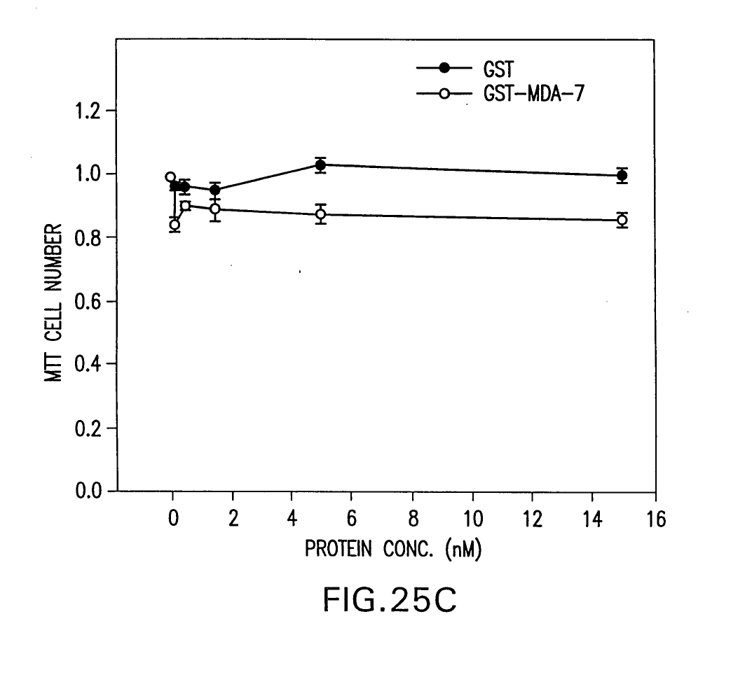

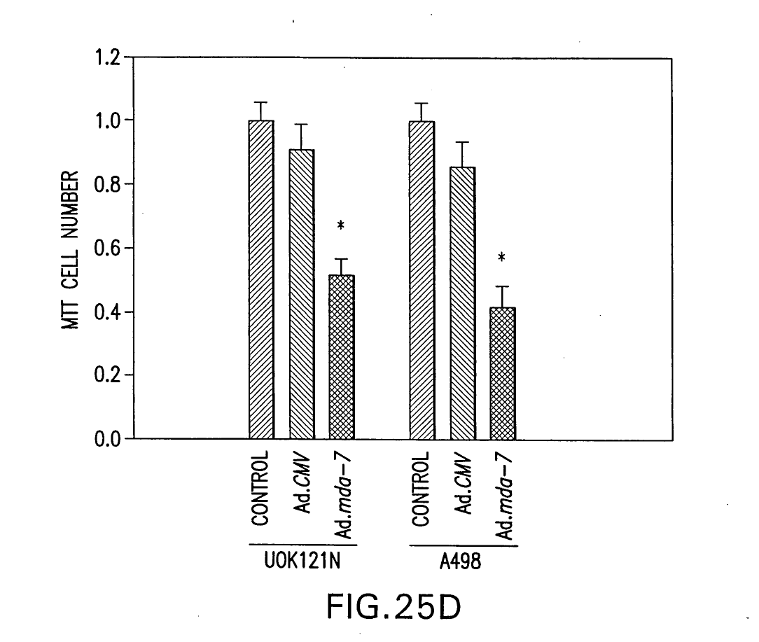

MDA-7 protein inhibits proliferation of RCC cell lines. Since RCC lines are resistant to adenoviral infection, MDA-7 was synthesized as a glutathione S-transferase (GST) fusion protein in E. coli and the inventors examined whether the purified MDA-7 protein could alter cell growth and survival in the two RCC cell lines A498 and UOK121N. GST-MDA-7, but not GST, caused a dose-dependent reduction in RCC line proliferation; this effect was not observed in primary renal epithelial cells (FIGS. 25A-25C).

To confirm that the effect of GST-MDA-7 was due to MDA-7 and not a contaminating bacterial protein, additional experiments were performed using MDA-7 protein synthesized in primary rodent hepatocytes. In these studies, cultures of primary rat hepatocytes were infected with Ad.mda-7 or a control virus and 96 h after infection the culture media from the hepatocytes was transferred into the culture media of RCC lines (FIG. 25D). RCC cells incubated with either fresh media or media from control virus-infected hepatocytes exhibited similar growth rates. However, kidney cancer cells incubated with media from Ad.mda-7-infected hepatocytes exhibited a significantly reduced growth rate.

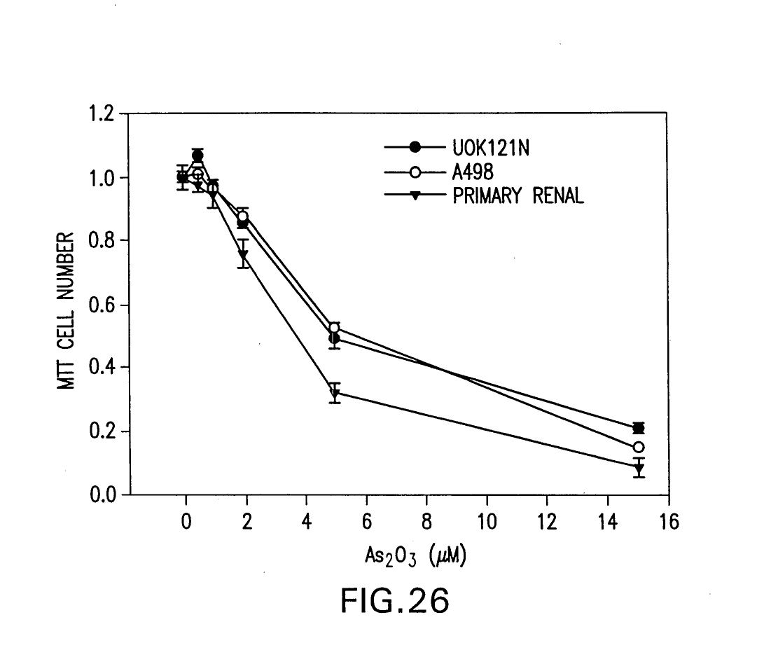

The free radical generator arsenic trioxide potentiates the anti-proliferative effects of MDA-7 in RCC cell lines. Arsenic trioxide is currently under investigation as an agent that can magnify the toxicity of established chemotherapeutic drugs (Dai et al., 2002, Cell Cycle 1: 143-152; Miller et al., 2002, Cancer Res. 62:3893-3903), presumed to be via the generation of free radical species in cells (Grad et al., 2001, Blood 98:805-813). Arsenic trioxide caused a dose-dependent reduction in the proliferation of A498, UOK121N and primary renal epithelial cells at concentrations above 1 μM (FIG. 26), which correlated with enhanced cell killing at higher concentrations. Previously, several groups have shown that ionizing radiation, which also generates free radicals, can interact with MDA-7 to enhance tumor cell killing (Kawabe et al., 2002, Mol. Ther. 6:637-644; Liu et al., 2002, Proc. Am. Assn. Cancer Res. 62:3209; Su et al., 2003 Oncogene 22:1164-1180). Thus the ability of arsenic trioxide to enhance the anti-proliferative and cell killing effects of MDA-7 in RCC lines was examined.

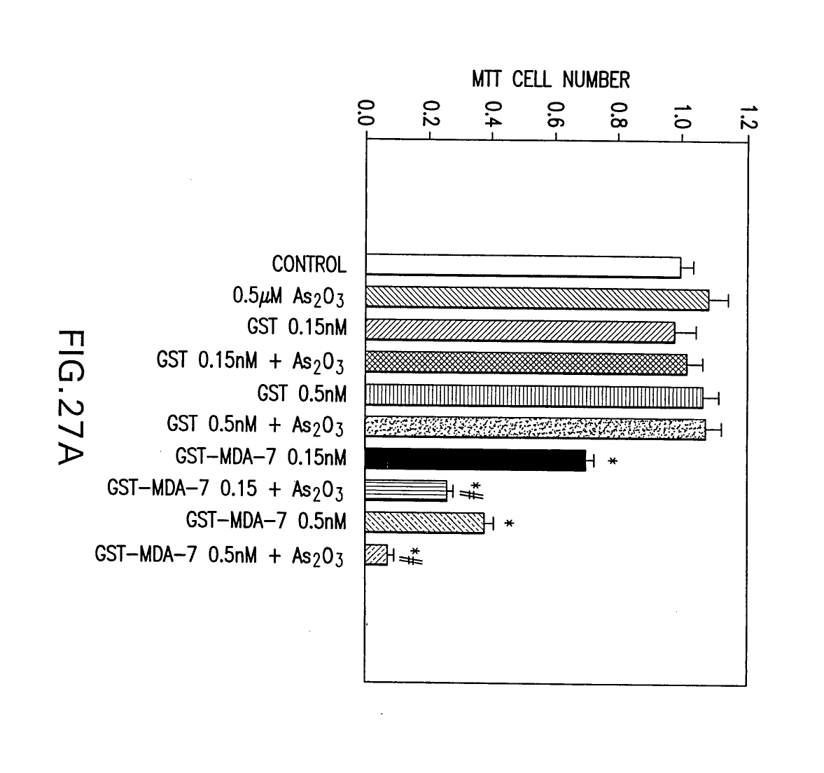

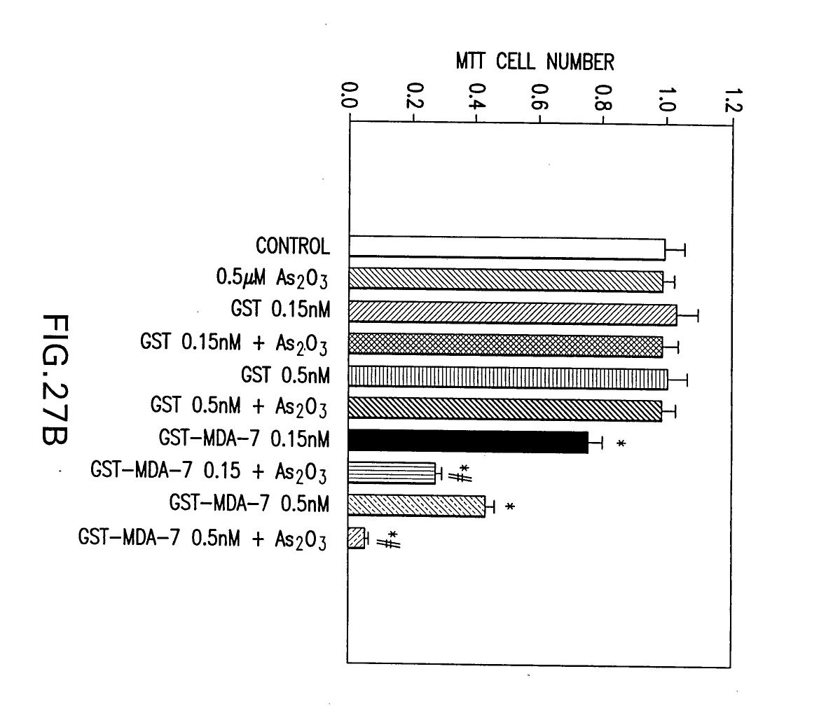

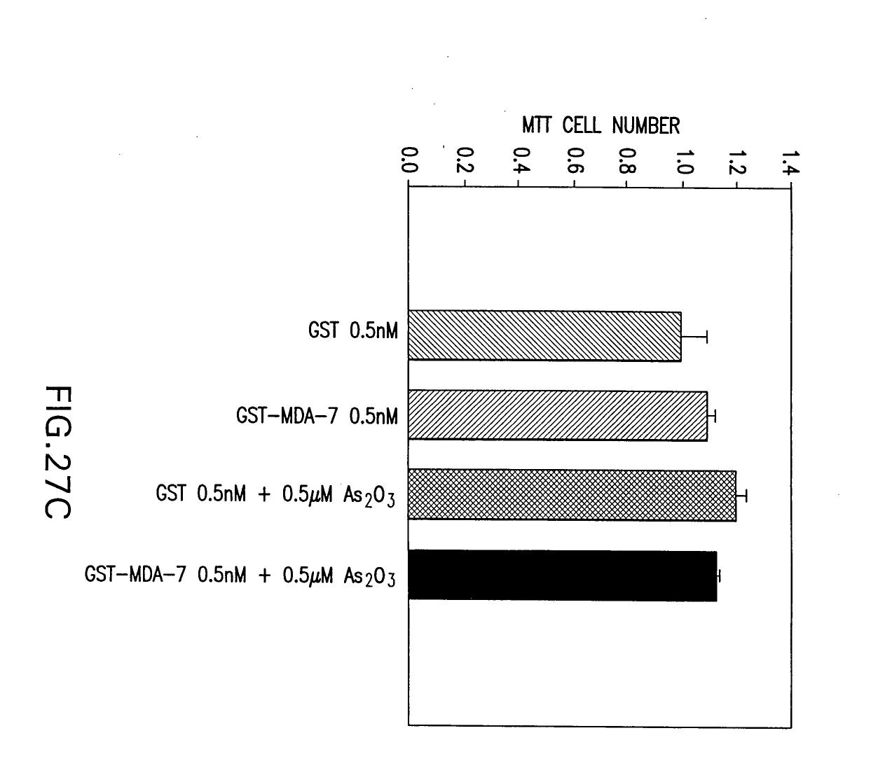

Arsenic trioxide enhanced the growth suppressive effects of GST-MDA-7, but not GST, in the RCC cell lines UOK121N and A498 (FIGS. 27A and 27B, respectively). Neither low concentrations of arsenic trioxide nor GST-MDA-7 alone altered the growth potential of primary renal epithelial cells (FIG. 27C).

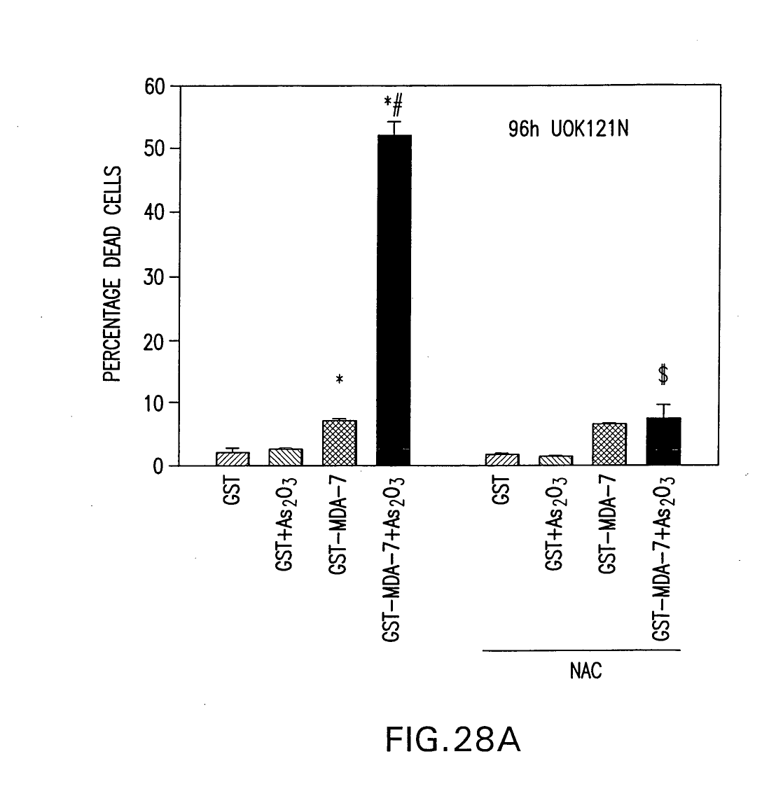

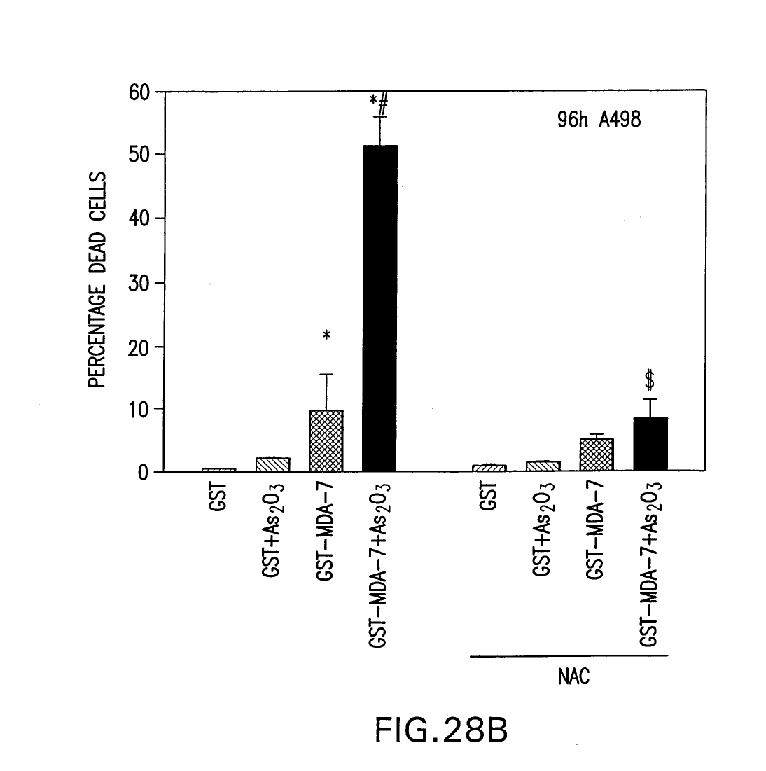

In parallel to the MTT proliferation assays in FIG. 27, the viability of cells was determined 96 h after GST-MDA-7/arsenic trioxide treatment. As judged by Wright Giemsa staining of fixed cells and microscopic examination of nuclear morphology, neither GST-MDA-7, arsenic trioxide nor their combination enhanced “classical” nuclear apoptosis. Cell nuclei appeared to be degraded in a non-apoptotic fashion, more indicative of necrosis. In general agreement with this finding significantly more trypan blue positive cells were present in cells treated with GST-MDA-7 and arsenic trioxide than under any other condition (FIG. 28). Arsenic trioxide is believed to enhance cell killing by the generation of free radicals, and incubation of cells with the free radical scavenger N-acetyl-cysteine significantly reduced cell killing (FIGS. 28A and 28B). The protective effect of N-acetyl-cysteine was also reflected in a blockade of enhanced growth suppression following arsenic trioxide and GST-MDA-7 treatment.

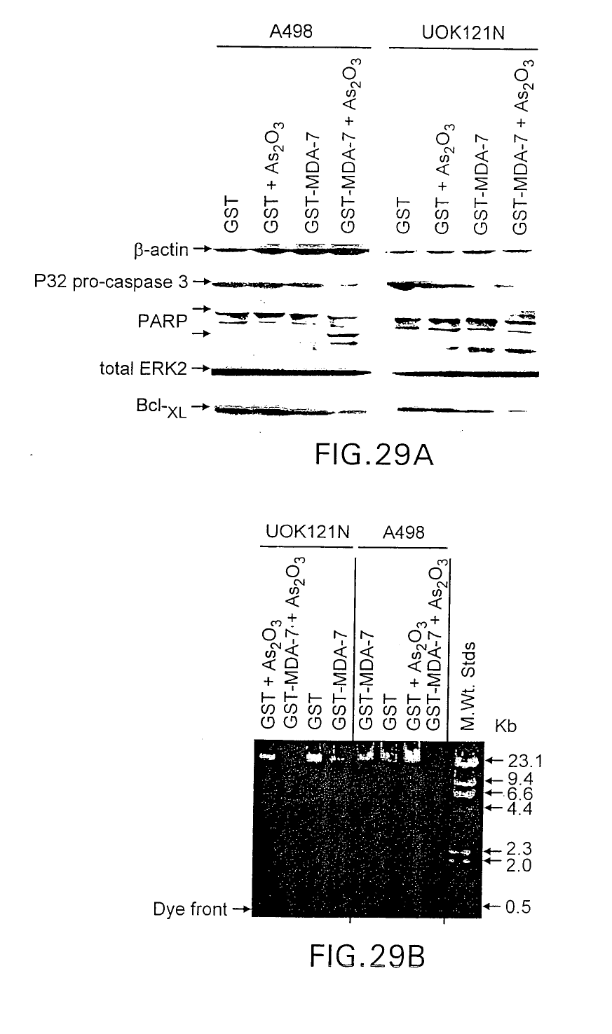

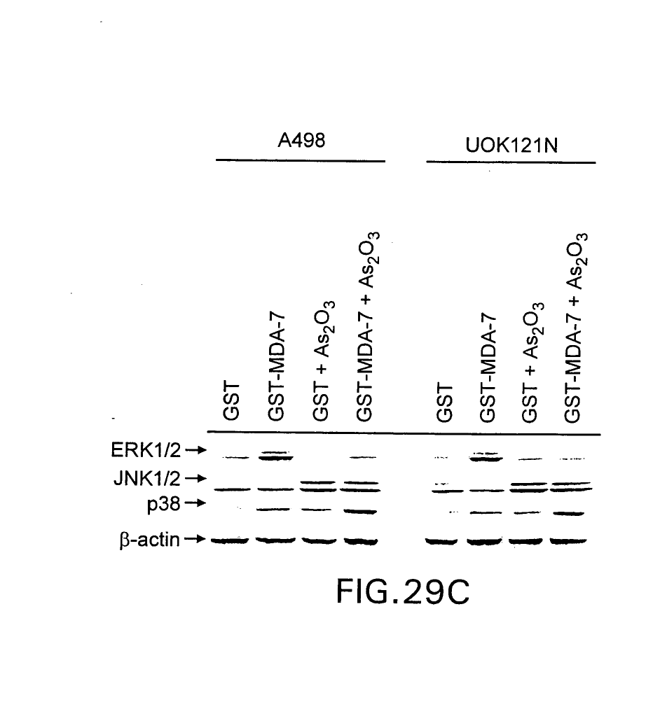

Treatment with the free radical generator arsenic trioxide and MDA-7 reduces levels of anti-apoptotic markers and activates the JNK1/2 pathway in RCC cell lines. The expression of pro-caspase 3, PARP and Bcl-x_{L}, as well as the integrity of nuclear DNA, was also examined after GST-MDA-7 and arsenic trioxide treatment. Despite only observing a weak enhancement in “classical” nuclear apoptotic morphology, p32 pro-caspase 3 and PARP were both cleaved and expression of Bcl-x_{L} was reduced (FIG. 29 A). Near-complete nucleosomal DNA degradation was observed 96 h following combined treatment with both agents, which was also suggestive of a necrotic form of cell death (FIG. 29B).

MDA-7 has been proposed to radio sensitize lung cancer cells by activation of the JNK1/2 pathway, whereas it has also been proposed to kill melanoma cells by activation of the p38 pathway (Kawabe et al., 2002, Mol. Ther. 6:637-644; Sarkar et al., 2002, Proc. Natl. Acad. Sci. U.S.A. 99:10054-10059). GST-MDA-7-induced growth arrest correlated with enhanced ERK1/2 and p38 activity whereas the arsenic trioxide enhancement of cell killing correlated with enhanced p38 and JNK1/2 activity and reduced ERK1/2 phosphorylation (FIG. 29C). Inhibition of JNK1/2 signaling using the JNK1/2/3 inhibitor SP600125 (10 μM), but not the p38α/β inhibitor SB203580 (2 μM), abolished the toxic interaction of GST-MDA-7 and arsenic trioxide.

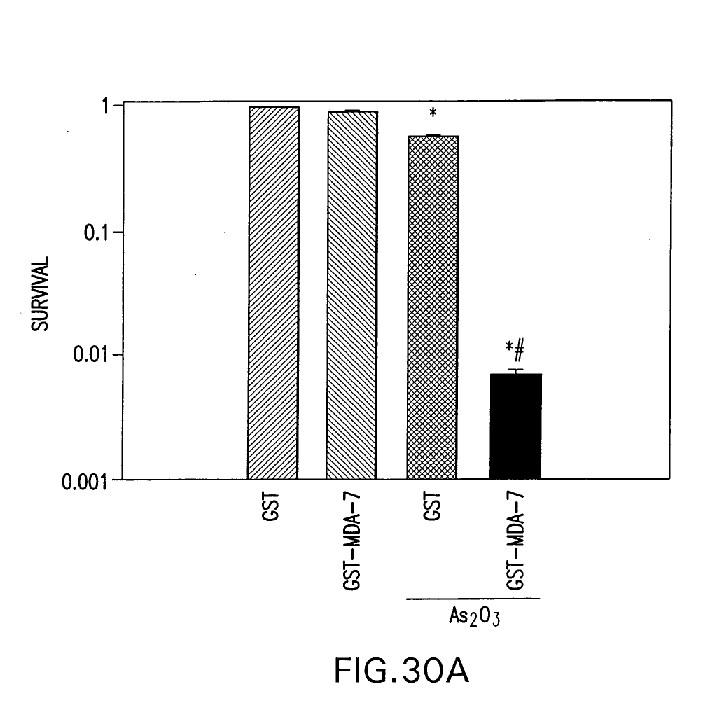

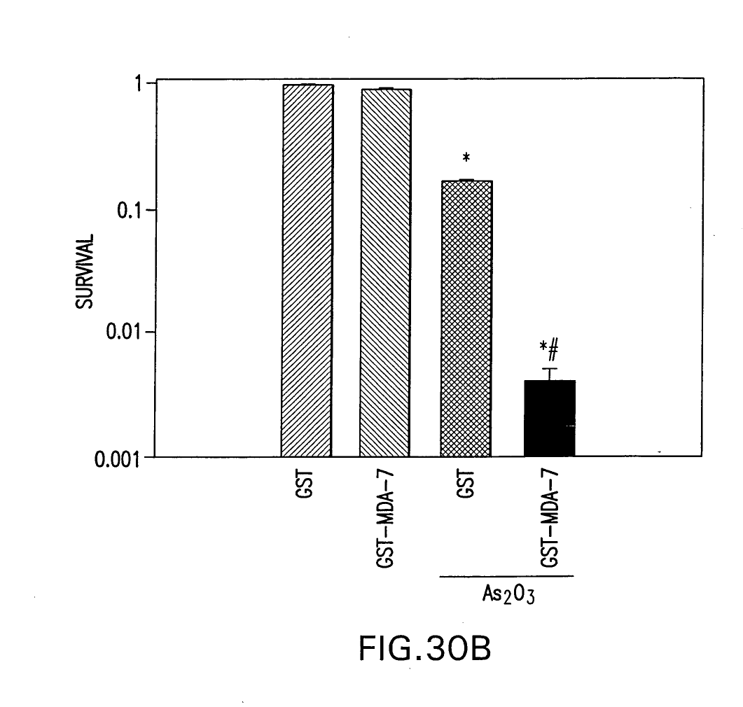

Treatment with the free radical generator arsenic trioxide and MDA-7 significantly reduces clonogenicity of RCC cell lines. GST-MDA-7 and arsenic trioxide significantly enhanced tumor cell death as judged by enhanced trypan blue inclusion in treated cells, and to confirm that this combination also reduced the long-term proliferative capacity of tumor cells, clonogenic survival assays were performed. Treatment of cells with low concentrations of GST-MDA-7 did not significantly alter long-term proliferation/cell survival whereas arsenic trioxide significantly reduced clonogenicity (FIGS. 30A and 30B). The combination of GST-MDA-7 with arsenic trioxide caused a large, greater than additive, reduction in the survival of renal carcinoma cells.

### 6.5. Example 5

### Generators of Free Radicals or Disruptors of Mitochondrial Membrane Potential Promote Killing of Cancer Cells By MDA-7

Materials and Methods

Cell Lines and Virus Infection Protocol. Human DU-145, PC-3 and LNCaP prostate carcinoma cells were obtained from the ATCC and cultured in RPMI 1640 supplemented with 10% FBS, 1% MEM sodium pyruvate and non-essential amino acids. P69, an SV40-immortalized human prostate epithelial cell line was provided by Dr. Joy Ware (Virginia Commonwealth University, VA) and grown under serum-free condition as described previously (Bae et al., 1994, Int. J. Cancer 58:721-729). Bcl-2 and Bcl-x_{L} stable over-expressing clones of each prostate cancer cell line were generated and cultured as described (Lebedeva et al., 2000, Cancer Research 60:6052-6060). The recombinant replication-defective Ad.mda-7 virus was created in two steps as described previously (Su et al., 1998, Proc. Natl. Acad. Sci. U.S.A. 95:14400-14405) and plaque purified by standard procedures. Cells were infected with 100 p.f.u./cell of Ad.mda-7 or Ad.vec (30 p.f.u./cell for LNCaP cells) and analyzed as described.

MTT Viability Assays. This method was performed as described (Lebedeva et al., 2000, Cancer Research 60:6052-6060). Briefly, cells were seeded in 96-well tissue culture plates (1.5×10³ cells per well) and treated as described in Results and Discussion. At the indicated time points, medium was removed, and fresh medium containing 0.5 mg/ml MTT was added to each well. The cells were incubated at 37° C. for 4 h and then an equal volume of solubilization solution (0.01N HCl in 10% SDS) was added to each well and mixed thoroughly. The optical density from the plates was read on a BioRad Microplate Reader Model 550 at 595 nm.

Annexin V Binding Assays. Cells were trypsinized, washed once with complete medium and stained with FITC-labeled Annexin-V (kit from Oncogene Research Products, Boston, Mass.) according to the manufacturer's instructions. Flow cytometry was performed immediately after staining.

Assessment of Mitochondrial ΔΨₘ and ROS Production. Changes in the inner mitochondrial transmembrane potential ΔΨₘ were determined by staining cells in 20 nM of DiOC₆(3) in PBS for 30 min at 37° C. in the dark. The dye accumulates in actively respiring mitochondria depending on ΔΨₘ (Zamzami et al., 1995, J. Exp. Med. 182:367-377). Controls were performed in the presence of 50 μM mitochondrial uncoupling agent mClCCP (Sigma). To determine ROS production, cells were stained with 2.5 μM HE or 5 μM DCFH-DA in PBS for 30 min at 37° C. in the dark (Castedo et al., 2002, J. Immunological Methods 265:39-47). Immediately after staining, cells were scored using FACScan flow cytometry (Becton-Dickinson, Mountain View, Calif.), and data were analyzed on CellQuest software, version 3.1 (Becton Dickinson). For inhibition experiments, NAC, CsA, BA (all from Sigma) or z-VAD.fmk (Calbiochem, La Jolla, Calif.) were added 2 h prior to infection with Ad.mda-7. In all cases, cells were gated to exclude cell debris.

Statistical Analysis. All of the experiments were performed at least three times. Results are expressed as mean±S.E. Statistical comparisons were made using an unpaired two-tailed Student's t-test. A p<0.05 was considered significant.

Results and Discussion

Ad.mda-7 Induces ROS and Apoptosis Selectively in Prostate Cancer Cells. Ad.mda-7 infection inhibits proliferation and induces apoptosis in diverse prostate cancer cell lines, but not in normal human prostate epithelial cells. Moreover, overexpression of anti-apoptotic members of the Bcl-2-family differentially protects prostate carcinoma cells from Ad.mda-7-induced apoptosis. The experiments described in this section employed these model systems to determine whether Ad.mda-7 regulates the levels of intracellular ROS and whether a rise in ROS is necessary for Ad.mda-7-mediated apoptosis.

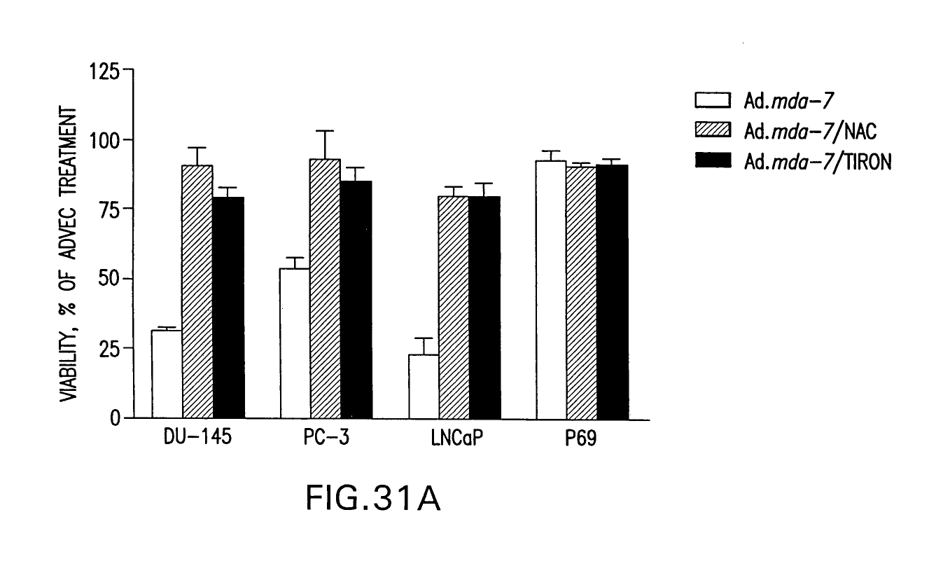

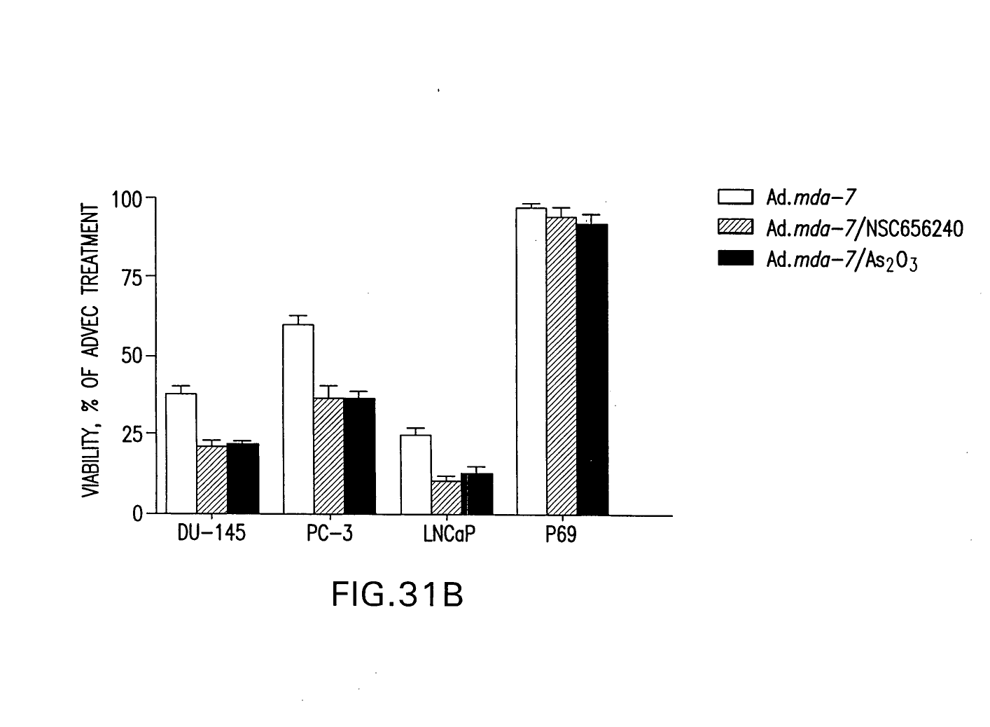

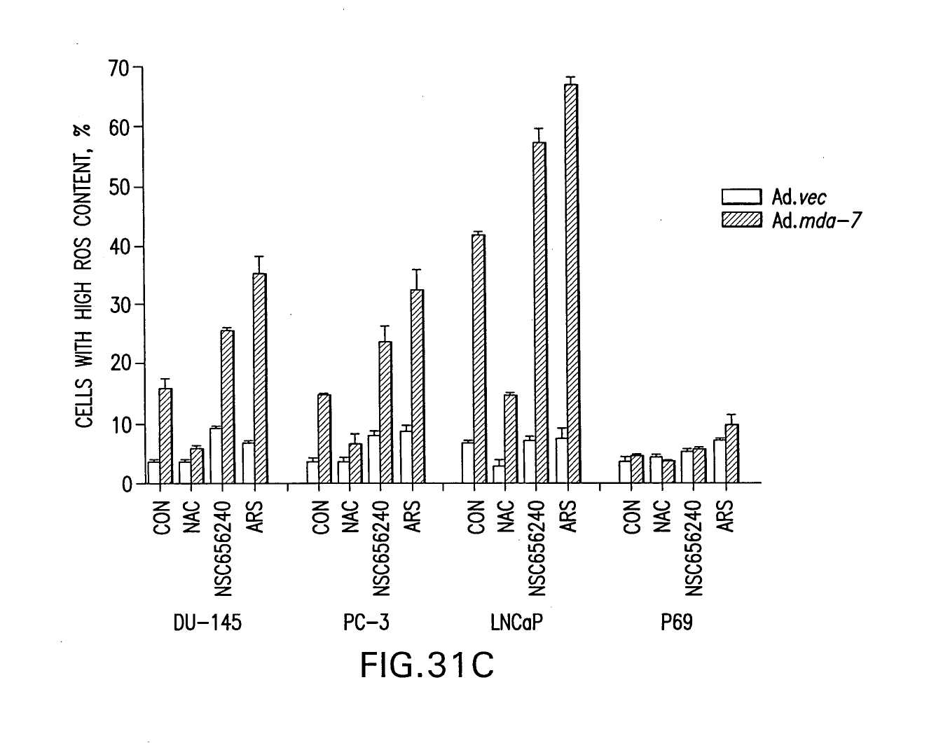

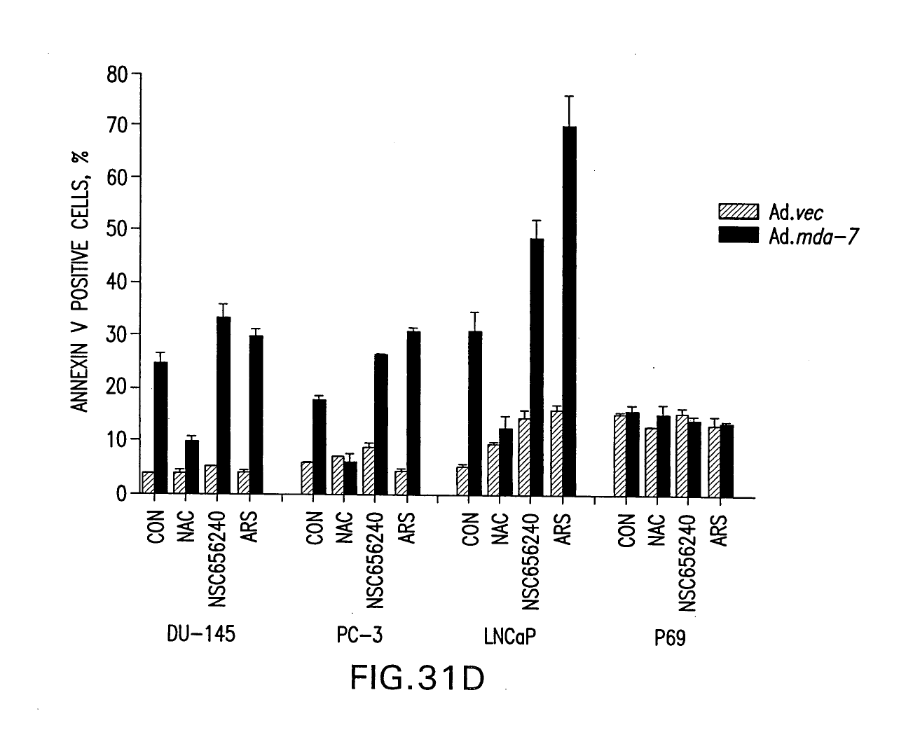

ROS (including singlet oxygen and hydrogen peroxide, as well as free radicals such as superoxide anion and hydroxyl radicals) regulate apoptosis and proliferation in response to a variety of stimuli, including tumor necrosis factor-α, ultraviolet and gamma irradiation, and anthracyclines (Jacobson et al., 1996, Trends Biochem. Sci. 21:83-86). To determine if ROS production contributes to apoptosis induction by Ad.mda-7 in prostate cancer cells, normal immortal prostate epithelial cells (P69) (Bae et al., 1994, Int. J. Cancer 58:721-729) and prostate carcinoma cells were infected with Ad.vec or Ad.mda-7 and the effect of antioxidants (NAC and Tiron) on Ad.mda-7-induced cell death was evaluated by MTT assays. In the presence of non-cytotoxic doses of a general antioxidant, NAC (5 mM), or a free radical and hydrogen peroxide scavenger, Tiron (1 mM), the cell death promoting activity of Ad.mda-7 was abrogated in all three prostate carcinoma cell lines (FIG. 31A). This inhibitory effect of antioxidant treatment was not associated with altered MDA-7 cellular or secreted protein levels. To explore the relationship between ROS and Ad.mda-7-induced apoptosis additional experiments were performed with arsenic trioxide (As₂O₃) and NSC656240, two agents that can promote ROS production in cancer cells (Dai et al., 1999, Blood 93:268-277; Jing et al., 1999, Blood 94:2102-2111) (FIG. 31B). Co-treatment of Ad.mda-7-infected prostate cancer cells with non-cytotoxic doses of As₂O₃ (10 μM) or NSC656240 (400 nM) potentiated cell death in all three prostate carcinoma cell lines, but not in normal immortal P69 prostate epithelial cells (FIG. 31B). These observations are consistent with contribution of free radicals Ad.mda-7-mediated induction of apoptosis in prostate carcinoma cells.

To confirm selective induction of ROS in prostate carcinoma cells following infection with Ad.mda-7, the levels of intracellular free radicals in P69 and prostate cancer cells were measured before and after Ad.mda-7 infection using two dyes: DCFH-DA and HE. Non-fluorescent DCFH-DA diffuses into cells, where it is deacetylated to DCF, which fluoresces upon reaction with hydrogen peroxide or nitrous oxide. HE enters the cell and can be oxidized by superoxide or free hydroxyl radicals to yield fluorescent ethidium (Castedo et al., 2002, J. Immunological Methods 265:39-47). Comparing these two dyes in prostate carcinoma cells indicated that Ad.mda-7-induced DCF fluorescence (indicating hydrogen peroxide and nitrous oxide production) to a somewhat greater extent than HE fluorescence (indicating free hydroxyl radical formation). Based on this consideration and because both values (DCF and HE fluorescence) were found to change coordinately, DCF fluorescence was used as a readout for ROS production. Flow cytometry analysis of cellular fluorescence revealed that Ad.mda-7 infection produced a 3- to 5-fold increase in ROS production in prostate carcinoma cells, but not in normal P69 cells (FIG. 31C). The largest ROS induction effect was apparent in LNCaP cells with comparable changes observed in PC-3 and DU-145 cells. Similarly, when treated with NSC656240 or As₂O₃, ROS levels increased proportionally in all of the prostate carcinoma cells with the greatest level of induction being apparent in LNCaP cells (FIG. 31C). Treatment with a non-cytotoxic dose of NAC suppressed Ad.mda-7-induced ROS generation, and also inhibited As₂O₃ and NSC656240 stimulated ROS generation in prostate cancer cells (FIG. 31C). The increase in ROS production coincided with apoptosis induction in the prostate cancer cell lines, as confirmed by Annexin V binding (FIG. 31D). As observed for ROS induction, pretreatment with non-toxic doses of NAC prevented Ad.mda-7-induced apoptosis. Moreover, a combination treatment with Ad.mda-7 plus As₂O₃ or NSC656240 increased apoptosis to variable extents in the three prostate cancer cell lines, without inducing apoptosis in normal P69 cells (FIG. 31D).

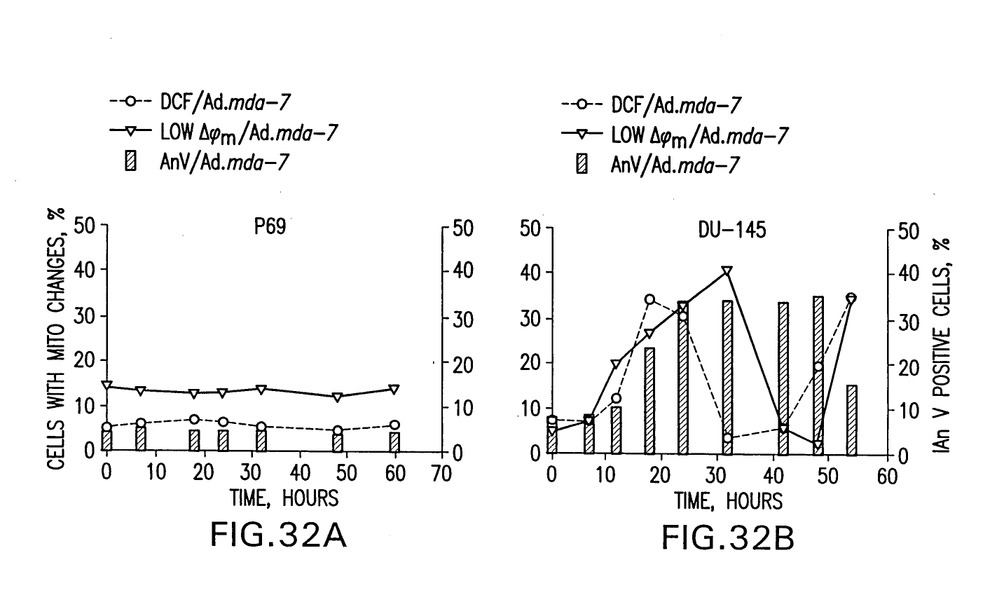

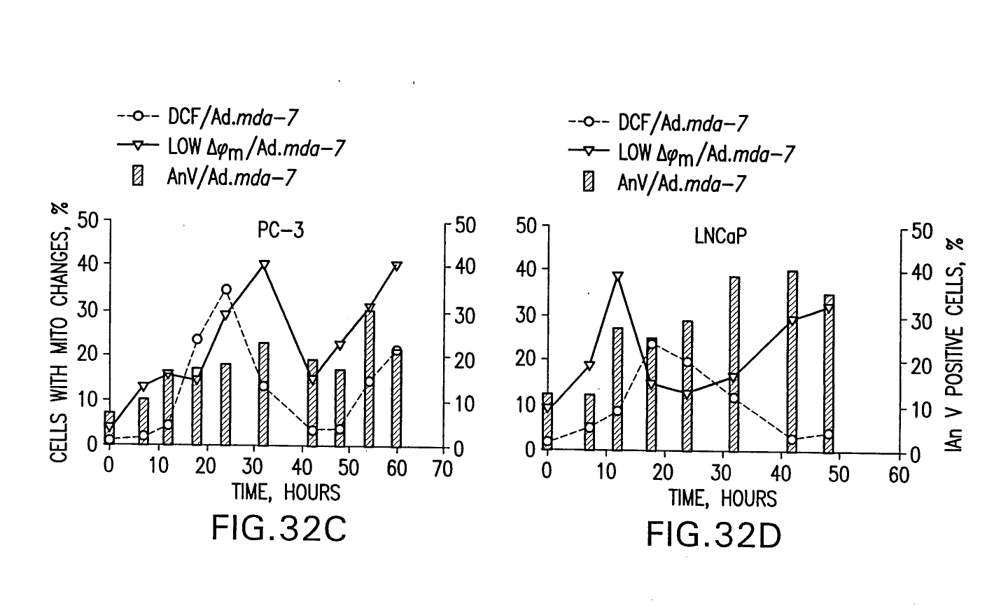

Ad.mda-7 Temporally and Selectively Induces ROS Production and ΔΨₘ Reduction in Prostate Carcinoma Cells. Because ROS may play a dual role in apoptosis, either being a modulator of mitochondrial membrane potential loss or a consequence of this change, depending on the death stimuli (Zamzami et al., 1995, J. Exp. Med. 182:367-377; Kroemer and Reed, 2000, Nat. Med. 6:513-519), the time course of mitochondrial changes (ROS, ΔΨₘ and membrane apoptotic changes (Annexin V binding) following Ad.vec or Ad.mda-7 infection were determined (FIG. 32). Cells were infected with Ad.vec or Ad.mda-7 at the indicated m.o.i., collected at different times up to 60 h and stained for ROS production with DCF-DA and with the cationic mitochondrial dye DiOC₆(3), which accumulates in active mitochondria, to determine changes in ΔΨₘ (Zamzami et al., 1995, J. Exp. Med. 182:367-377). In parallel, annexin-V binding assays were performed as described in Methods. The data in FIG. 32 documents the effects of Ad.mda-7 on mitochondrial and cytoplasmic apoptotic changes in Ad.mda-7-infected normal P69 and prostate carcinoma cells. A detailed analysis of the time course of ROS production and ΔΨₘ changes confirmed that in all three prostate cancer cell lines, the initial ΔΨₘ decrease occurred prior to ROS induction. The initial small decrease in ΔΨₘ at early times (6-8 hours) was followed by a ROS production increase (10-20 h). The decline in ΔΨₘ continued up to 12 hr in LNCaP and up to 30 hr in DU-145 and PC-3. At 45-50 hr, a secondary burst of ROS production and a concurrent final steep drop in ΔΨₘ occurred, documenting complete mitochondrial dysfunction. As shown in FIG. 32, the decline in ΔΨₘ and the increase in Annexin V binding (an early indicator of cytoplasmic apoptosis) occurred concomitantly. Thymocytes undergoing glucocorticoid-induced death exhibit a reduction in ΔΨₘ preceding exposure of phosphatidyl serine (PS) residues on the plasma membrane, enhanced generation of superoxide anions, and nuclear degradation (Zamzami et al., 1995, J. Exp. Med. 182:367-377). The present studies suggest that Ad.mda-7 induced apoptosis may follow a similar chronology. It is worth noting that in all prostate cancer cell lines, there was a correlation between mitochondrial changes and MDA-7 protein expression. Mitochondrial changes in Ad.mda-7-infected prostate cancer cells first became apparent when MDA-7 protein was initially detected by immunoblotting. In a previous study, it was demonstrated that MDA-7 protein first appeared by 6-9 h following Ad.mda-7 infection of prostate cancer cells. However, despite similar kinetic changes in MDA-7 protein expression in Ad.mda-7 infected normal prostate epithelial cells, no decline in survival or mitochondrial functions or induction of apoptosis were evident (FIG. 32).

Ad.mda-7 Infection Modulates Mitochondrial Permeability Transition (MPT) in Prostate Cancer Cells. Since ROS production and the decline in ΔΨₘ were directly associated with apoptosis or reduced cell survival in prostate carcinoma cells infected with Ad.mda-7 (FIG. 32), the role of MPT in Ad.mda-7-induced apoptosis was investigated. MPT is characterized by the opening of mitochondrial megachannels to allow solutes and water to enter the mitochondria (Zoratti and Szabo, 1995, Biochim. Biophys. Acta 1241:139-176). MPT can be triggered by ROS or other agents resulting in a decrease in ΔΨₘ, followed by depletion of ATP or activation of caspases/endonucleases (rev. in Kroemer and Reed, 2000, Nat. Med. 6:513-519). This process is controlled by a multiprotein complex found in the inner and outer membranes of the mitochondria known as the permeability transition pore (PTP) (Zoratti and Szabo, 1995, Biochim. Biophys. Acta 1241:139-176). The PTP consists of VDAC/porin, ANT, cyclophilin D, the complex forming the peripheral benzodiazepine receptors (PBzR) and other proteins (Zoratti and Szabo, 1995, Biochim. Biophys. Acta 1241:139-176). Upon PTP opening, the mitochondria lose their ΔΨₘ across the inner membrane culminating in apoptosis accompanied by an immediate shutdown of mitochondrial biogenesis. Another consequence of ΔΨₘ disruption is the uncoupling of oxidative phosphorylation (Vayssiere et al., 1994) and the generation of superoxide anion on the uncoupled respiratory chain resulting in further damage to proteins and membranes (Zamzami et al., 1995, J. Exp. Med. 182:367-377).

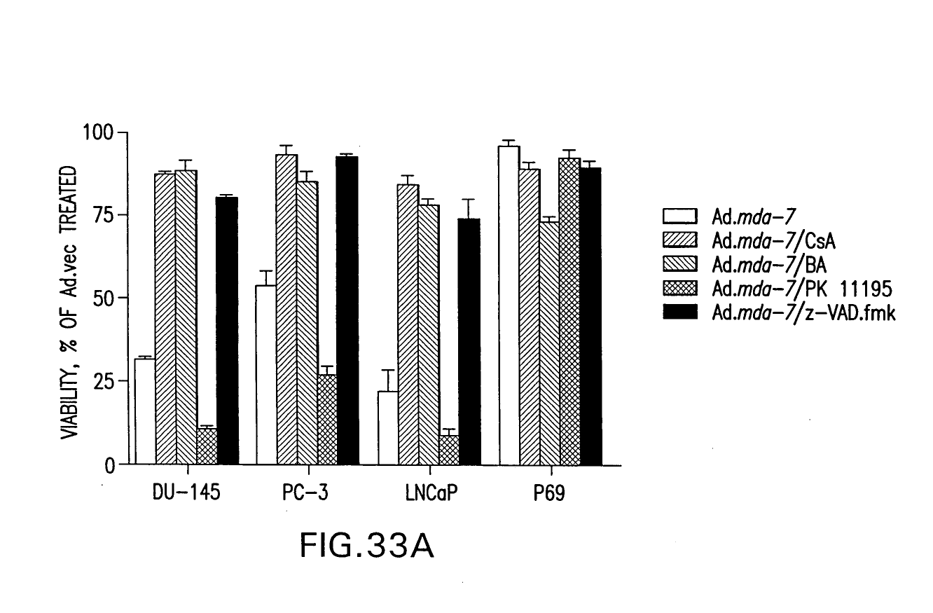

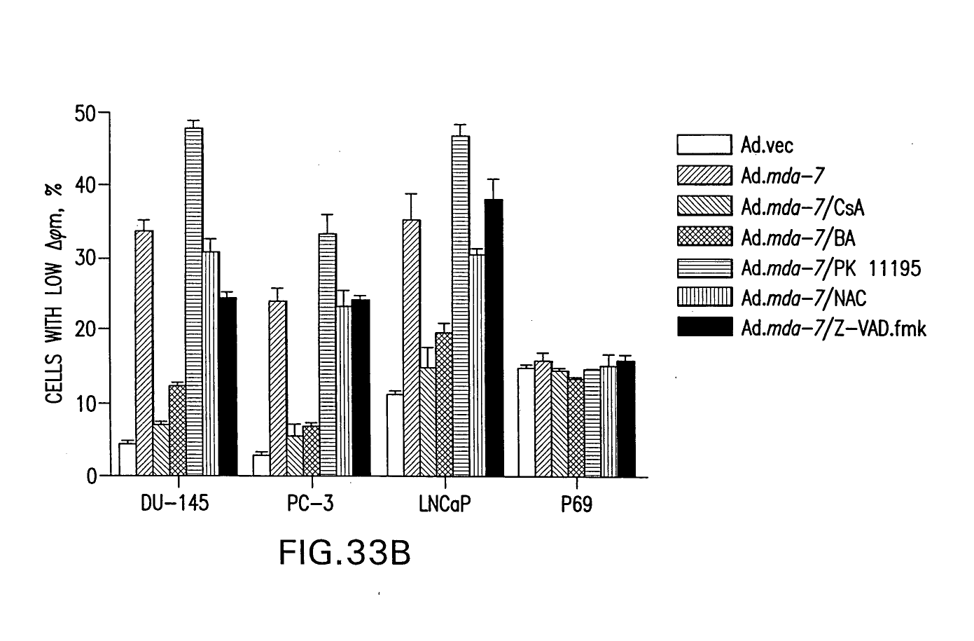

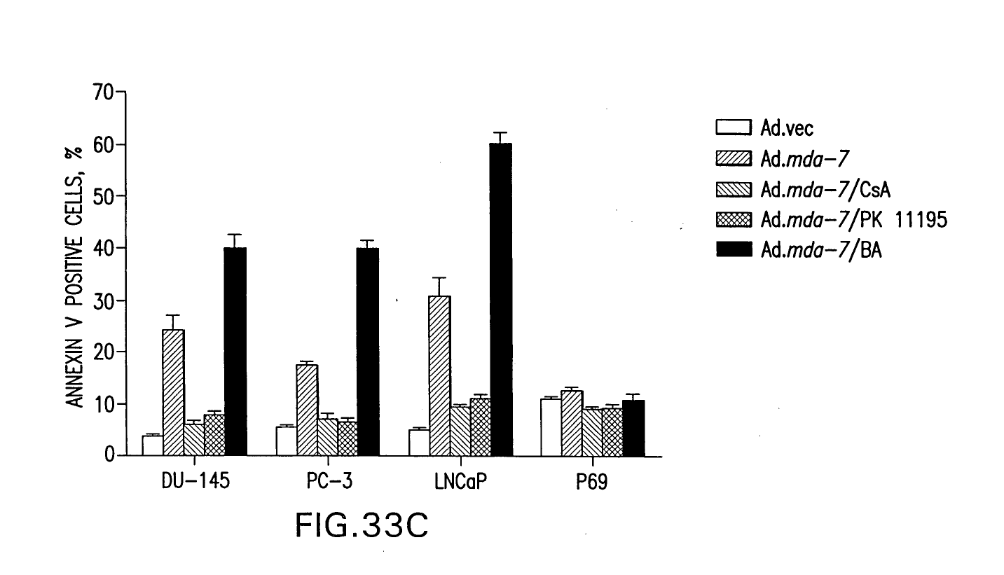

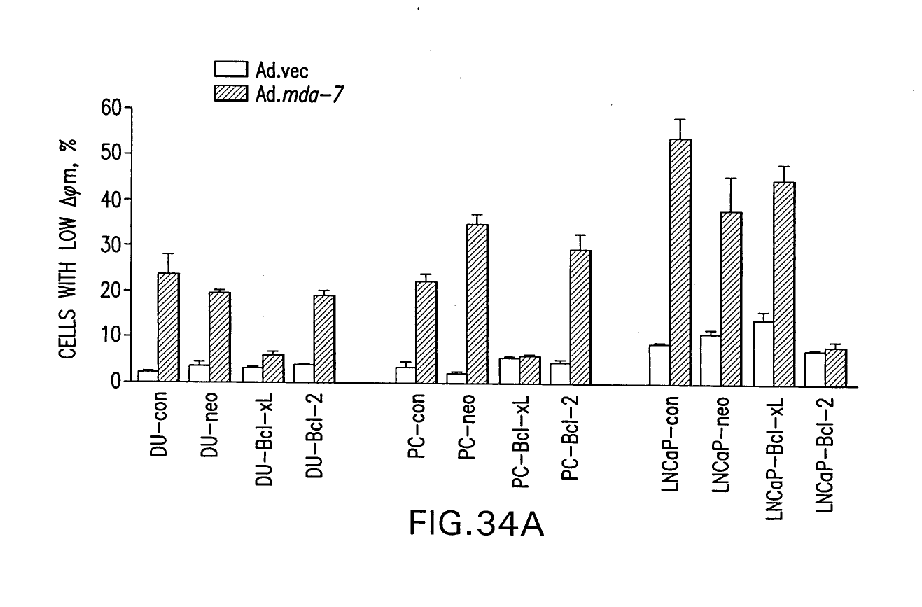

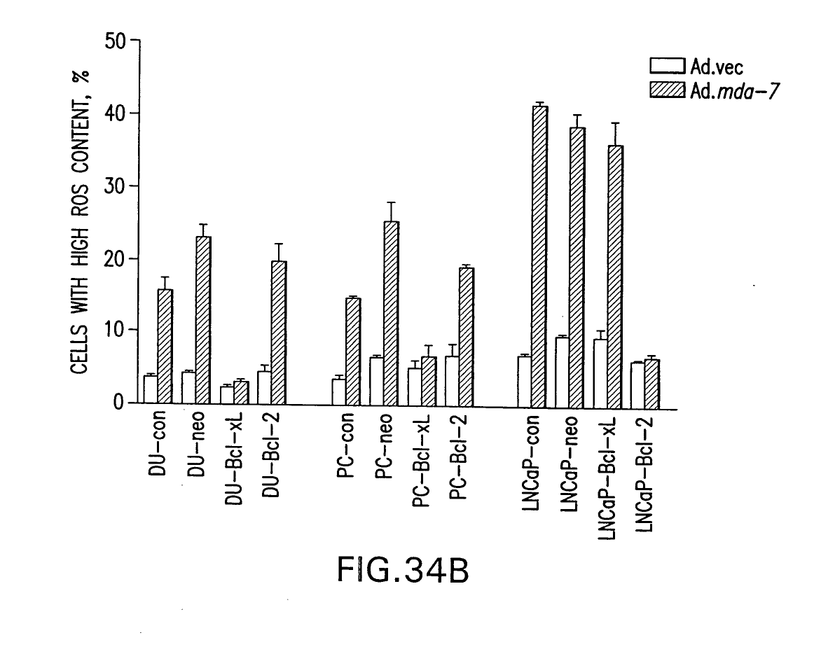

CsA and BA specifically bind to different components of the PTP complex (cyclophilin D and ANT, respectively), thereby preventing mitochondrial membrane permeabilization and apoptosis in a wide variety of cell types (Klingenberg et al., 1970, Biochem. Biophys. Res. Commun. 39:344-351; Crompton et al., 1988, Biochem. J. 255:357-360; Marchetti et al., 1996, J. Exp. Med. 184:1155-1160; Zamzami et al., 1995, J. Exp. Med. 182:367-377). On the other hand, the PBzR agonist (PK11195) can potentiate the induction of MPT (Pastorino et al., 1994). Based on these considerations, prostate cancer cells and normal P69 cells were pretreated with non-toxic doses of CsA (200 nM), BA (50 μM) or PK11195 (50 μM) for 2 h post-infection with Ad.vec or Ad.mda-7 and cellular viability and early (cytoplasmic) apoptosis were assessed 18 h (LNCaP cells) and 24 h (DU-145, PC-3 and P69 cells) post-infection. Pretreatment with CsA or BA prevented cell death (FIG. 33A) and annexin V exposure in Ad.mda-7-infected prostate cancer cells (FIG. 33C). Analysis of mitochondrial changes (DiOC₆(3) retention) and ROS production (DCF-DA staining) confirmed that CsA and BA abrogated the decline in ΔΨₘ (FIG. 33B). In a recent study employing two lung cancer cell lines, H1299 and A549, Ad.mda-7 induced changes in ΔΨₘ only in A549 cells and CsA did not prevent Ad.mda-7-induced cell death in either of these cell types or alter ΔΨₘ in A549 cells (Pataer et al., 2003, J. Thorac. Cardiovasc. Surg. 125:1328-1335). The reason for these differences between prostate carcinoma cells and the two lung carcinoma cell lines are not currently known, but could reflect inherent differences in the mode of action of mda-7/IL-24 in these two tumor cell types or simply differences in experimental protocols, i.e. the dose of CsA used, the temporal kinetics of changes investigated and/or a different protocol to measure ΔΨₘ (Castedo et al., 2002, J. Immunological Methods 265:39-47). PK11195 increased MPT, annexin V staining and enhanced Ad.mda-7-induced killing in DU-145, PC-3 and LNCaP, without inducing any of these changes in P69 cells (FIGS. 33A, 33B, 33C). The ability of CsA and BA to inhibit ΔΨₘ decline, ROS production, and apoptosis and the ability of a PBzR agonist to promote these changes highlights the importance of mitochondria and MPT as arbiters of Ad.mda-7-induced death in prostate carcinoma cells (FIG. 34C).

In contrast to its ability to prevent Ad.mda-7-induced decreases in cell viability up to 48 h (FIG. 31A) and induction of ROS (FIG. 31C) in all three prostate cancer cell lines, when evaluated at 18 h (LNCaP) and 24 h (DU-145, PC-3 and P69), NAC only minimally inhibited Ad.mda-7-induced ΔΨₘ in DU-145 and LNCaP cells without affecting this parameter in PC-3 cells (FIG. 33B). Similarly, whereas the general caspase inhibitor z-VAD.fmk inhibited cell death in all three prostate cancer cells (FIG. 33A) it only partially blocked Ad.mda-7-induced ΔΨₘ in DU-145 cells without affecting ΔΨₘ in PC-3 or LNCaP cells (FIG. 3B), suggesting that Ad.mda-7 may initially facilitate mitochondrial changes via a caspase-independent mechanism (Marchetti et al., 1996, J. Exp. Med. 184:1155-1160; Zamzami et al., 1996, J. Exp. Med. 183:1533-1544).

Bcl-2 and Bcl-x_{L} Differentially Protect Prostate Cancer Cells from ROS Induction and Decreased ΔΨₘ Following Infection with Ad.mda-7. Over-expression of Bcl-2 and Bcl-x_{L} can differentially protect prostate carcinoma cells from apoptosis induced by Ad.mda-7. These bcl-2-family members have also been found to protect diverse cell types from ROS-dependent (Kane et al., 1993, Science 262:1274-1277; Hockenbery et al., 1993, Cell 75:241-251) and ROS-independent (Weil et al., 1996, J. Cell Biol. 133:1053-1059) apoptosis. Extensive research on the mechanism of inhibition of apoptosis by Bcl-2 has focused on its interaction with and regulation of mitochondrial function, particularly the mitochondrial permeability (MP) pore (Vander Heiden et al., 1997, Cell 91:627-637; Gottlieb et al., 2000, Mol. Cell. Biol. 20:5680-5689). However, the localization of Bcl-2 to intracellular sites of oxygen free radical generation, including the cytoplasmic face of the mitochondrial outer membrane, endoplasmic reticulum and nuclear membranes predict that Bcl-2 may exhibit antioxidant properties (Hockenbery et al., 1993, Cell 75:241-251). Furthermore, Bcl-2 knockout mice express a phenotype consistent with that of mice exposed to chronic oxidative stress (polycystic kidney disease and follicular hypopigmentation) (Veis et al., 1993, Cell 75:229-240).

To examine the relationship between Bcl-2-family members and changes in mitochondrial function and ROS, a series of well-characterized prostate cancer cell clones displaying stable overexpression of Bcl-2 or Bcl-x_{L} were employed. In DU-145 and PC-3 cells, over-expression of Bcl-x_{L} and not Bcl-2 prevented Ad.mda-7-induced apoptosis, whereas in LNCaP cells over-expression of Bcl-2 and not Bcl-x_{L} was protective. As shown on FIG. 34A, over-expression of Bcl-x_{L} completely abrogated the decline in ΔΨₘ in DU-145 and PC-3 cells following Ad.mda-7 infection, without preventing this change in LNCaP cells. In contrast, Bcl-2 over-expression, but not Bcl-x_{L} over-expression prevented the reduction in ΔΨₘ in LNCaP cells upon Ad.mda-7 infection. Similarly, Bcl-x_{L}, but not Bcl-2, protected DU-145 and PC-3 cells from Ad.mda-7-induced ROS induction, whereas Bcl-2, but not Bcl-x_{L}, protected LNCaP cells from this biochemical change (FIG. 34B). These studies provide further confirmation between induction of apoptosis in prostate cancer cells by Ad.mda-7 and changes in mitochondrial function (reduction in ΔΨₘ) and ROS production.

In the apoptotic process, Ad.mda-7 induces mitochondrial depolarization, which is associated with MPT and this process is inhibited by simultaneous treatment with ROS inhibitors, NAC and Tiron, and potentiated by simultaneous treatment with ROS inducers, As₂O₃, NSC656240 and the PBzR agonist PK11195. The ability of Ad.mda-7 to induce a loss in Δψₘ and enhance ROS production is inhibited, as is its ability to induce apoptosis, in specific prostate cancer cells by forced over-expression of anti-apoptotic members of the Bcl-2-gene family, Bcl-2 or Bcl-x_{L}. These experiments document a relationship between mitochondrial dysfunction and ROS induction and sensitivity to apoptosis induction by mda-7/IL-24 in prostate cancer cells, which are consistent with the observation that ability of Ad.mda-7 to selectively kill prostate cancer cells can be augmented by agents that enhance mitochondrial dysfunction and induce ROS production.

### 6.6. Example 6

### Free Radical Generators in Combination with mda-7 Nucleic Acid or MDA-7 Protein as a Treatment for Ovarian Cancer

Materials and Methods

SKOV3 cells (ATCC, Manassas Va.) were plated at 10,000 per well in 24 well plates. 24 h after plating cells were infected with either control virus or Ad.mda-7 virus at increasing multiplicities of viral infection. 24 h after infection, cells were treated with 500 nM 4-HPR. 96 h after 4-HPR addition, cell numbers were determined using MTT assays as described above.

Results

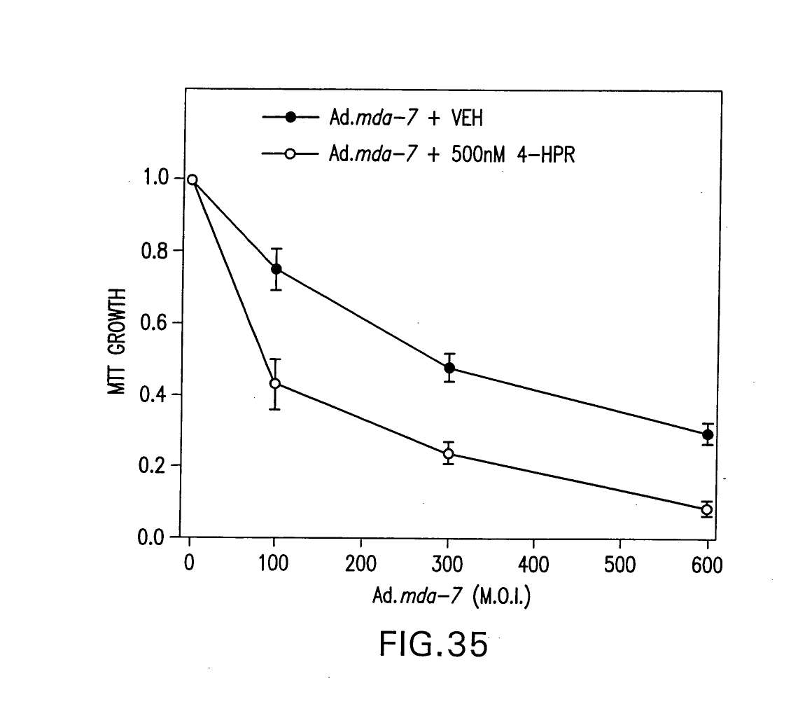

The results of this study are shown in FIG. 35. The data presented are corrected for 4-HPR toxicity (as a single agent) and corrected for any anti-proliferative effect of control viral infection. As shown in FIG. 35, 4-HPR potentiated the toxic effects of MDA-7 expression in ovarian cancer cell. 4-HPR did not synergize with control virus to suppress growth of this same cell line.

### 6.7. Example 7

### Free Radical Generators in Combination with mda-7 Nucleic Acid or MDA-7 Protein as a Treatment for Ovarian Cancer

Materials and Methods

Cell Lines and Virus Infection Protocol. Human PANC-1, MIA PaCa-2, AsPC-1 and BxPC-3 pancreatic carcinoma cells were obtained from the ATCC and cultured in RPMI 1640 supplemented with 10% FBS. Human immortalized astrocytes and human immortalized melanocytes were obtained as described earlier (Lebedeva et al., 2002, Oncogene 21:708-718; Su et al., 2003, Oncogene 22:1164-1180) cultured in DMEM medium. The recombinant replication-defective Ad.mda-7 virus was created in two steps as described previously (Su et al., 1998, Proc. Natl. Acad. Sci. U.S.A. 95:14400-14405) and plaque purified by standard procedures. Cells were infected with 100 p.f.u./cell of Ad.mda-7 or Ad.vec and analyzed as described.

MTT Viability Assays. Cell viability was assessed by MTT assays as described (Lebedeva et al., 2000, Cancer Research 60:6052-6060). Briefly, cells were seeded in 96-well tissue culture plates (1.5×10³ cells per well) and treated with various agents. At the indicated times, medium was removed, and fresh medium containing 0.5 mg/ml MTT was added to each well. The cells were incubated at 37° C. for 4 h and then an equal volume of solubilization solution (0.01N HCl in 10% SDS) was added to each well and mixed thoroughly. The optical density from the plates was read on a BioRad Microplate Reader Model 550 at 595 nm.

Annexin V Binding Assays. Cells were trypsinized, washed once with complete medium and stained with FITC-labeled Annexin-V (kit from Oncogene Research Products, Boston, Mass.) according to the manufacturer's instructions. Flow cytometry was performed immediately after staining.

Cell Cycle Analysis. Cells were trypsinized, washed 2× with PBS and fixed in 70% ethanol overnight at −20° C. Then cells were washed 2 times with PBS, and aliquots of 1×10⁶ cells were resuspended in 1 ml of PBS containing 1 mg/ml of RNase A and 0.5 mg/ml of propidium iodide. After 30 min incubation, cells were analyzed by flow cytometry using a FACScan flow cytometer (Becton Dickinson, San Jose, Calif.).

Preparation of Cell Extracts and Western Blotting Analysis. Cells were washed 2× with cold PBS and lysed on ice for 30 min in 100 l of cold RIPA buffer [50 mM Tris-HCl (pH 8.0), 150 mM NaCl, 0.1% SDS, 1% NP40, and 0.5% sodium deoxycholate] with freshly added 0.1 mg/ml phenylmethylsulfonyl fluoride, 1 mM sodium orthovanadate, and 1 mg/ml aprotinin. After cell debris were removed by centrifugation at 14,000 g for 10 min at 4° C., protein concentrations were determined using the Bio-Rad protein assay system (Bio-Rad Laboratories, Richmond, Calif.). Aliquots of cell extracts containing 20-50 mg of total protein were resolved in 12% SDS-PAGE and transferred to Immobilon-P PVDF membranes (Millipore Corp., Bedford, Mass.). Filters were blocked for 1 h at room temperature in Blotto A [5% nonfat milk powder in TBS-T: 10 mM Tris-HCl (pH 8.0), 150 mM NaCl, 0.05% Tween 20], and then incubated for 1 h at room temperature in Blotto A containing a 1:1000 dilution of rabbit anti MDA-7 polyclonal antibody. After washing in TBS-T buffer (3×5 min, RT), filters were incubated for 1 h at room temperature in Blotto A containing a 1:10,000 dilution of peroxidase conjugated anti-rabbit secondary antibody (Amersham, Arlington Heights, Ill.). After washing in TBS-T, ECL was performed according to the manufacturer's recommendation.

Assessment of ROS Production. To determine ROS production, cells were stained with 2.5 μM HE or 5 μM DCFH-DA in PBS for 30 min at 37° C. in the dark. Immediately after staining, cells were analyzed by flow cytometry (FACSscan, Becton-Dickinson, Mountain View, Calif.), and data were analyzed using CellQuest software, version 3.1 (Becton Dickinson). For inhibition experiments, NAC (Sigma) was added 2 h prior to infection with Ad.mda-7. In all cases, cells were gated to exclude cell debris.

Statistical Analysis. All of the experiments were performed at least three times. Results are expressed as mean±S.E. Statistical comparisons were made using an unpaired two-tailed Student's t-test. A P<0.05 was considered significant.

Results

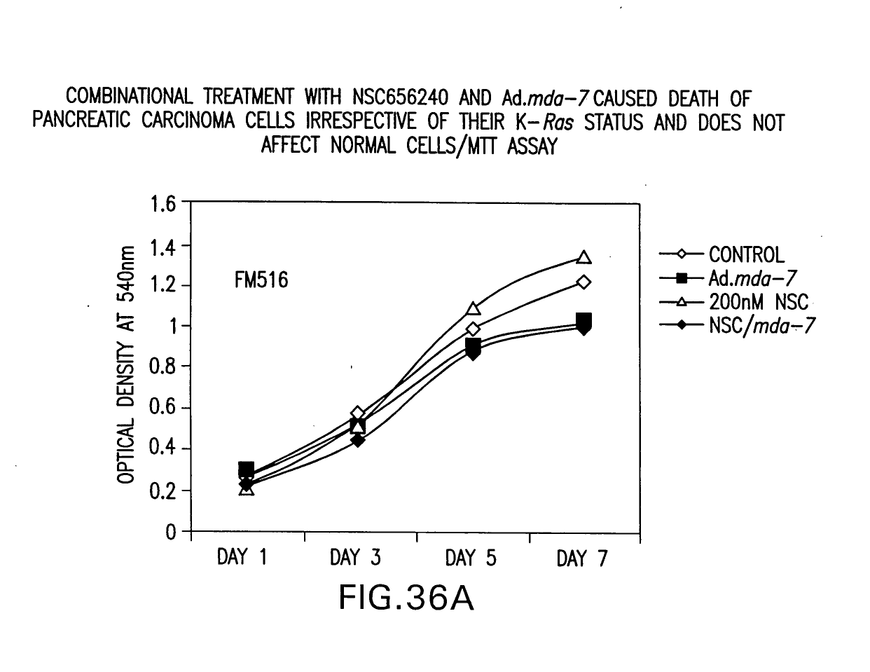

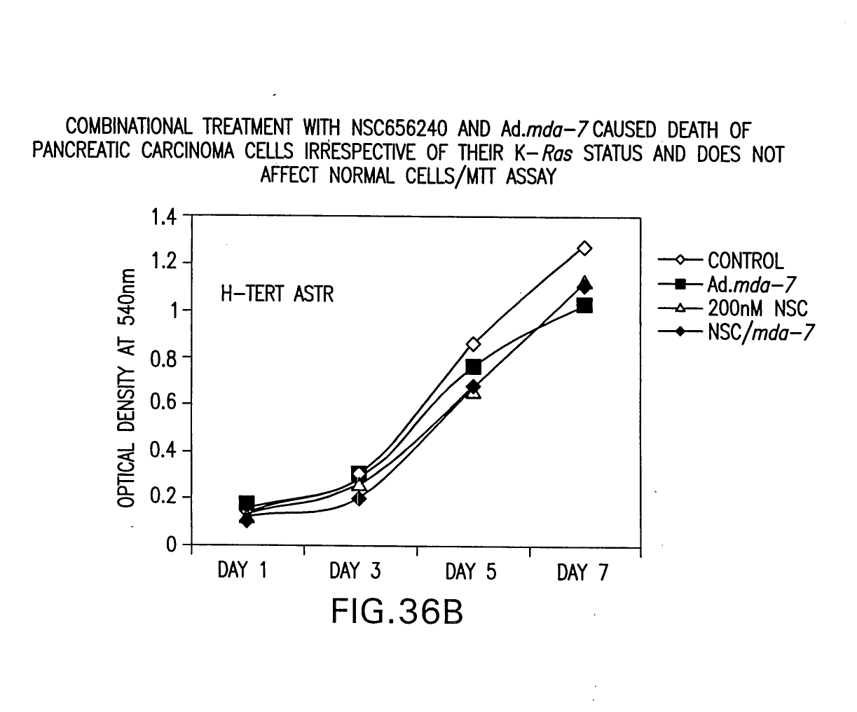

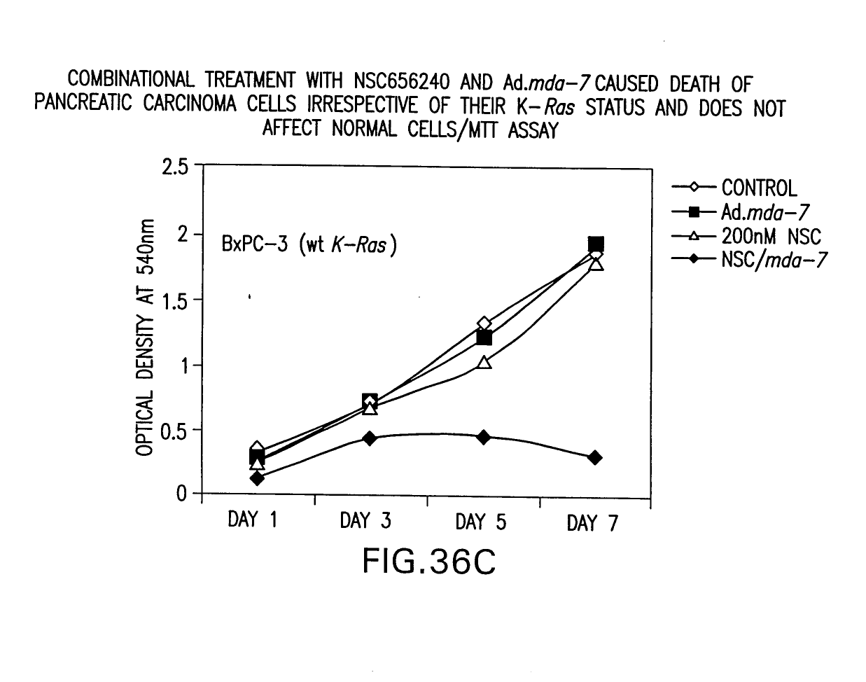

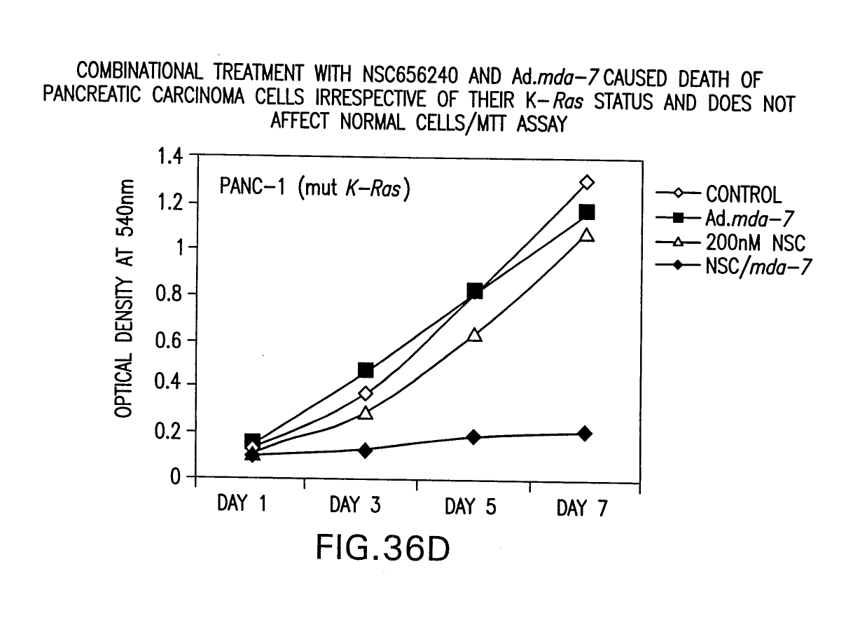

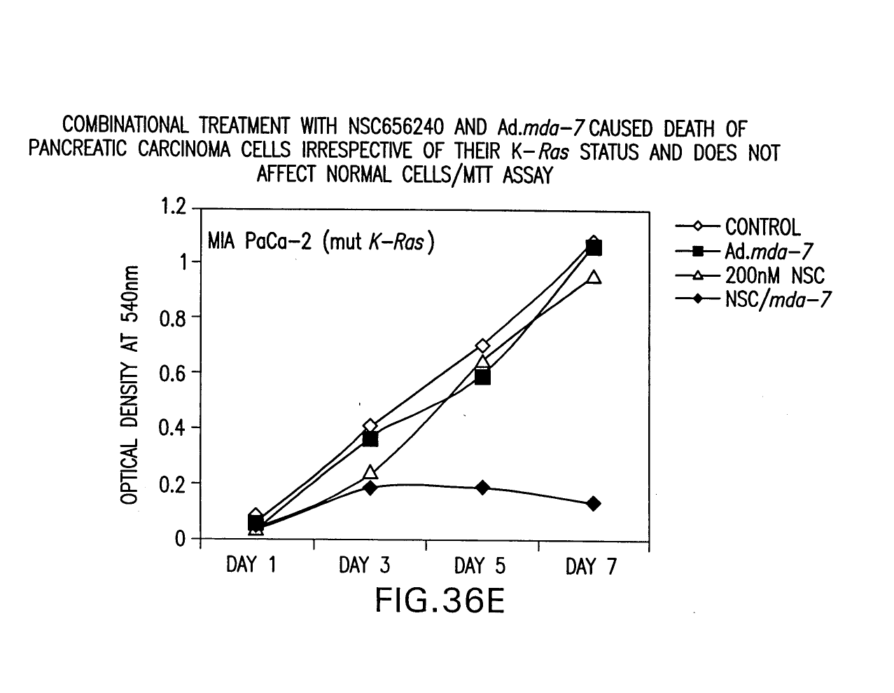

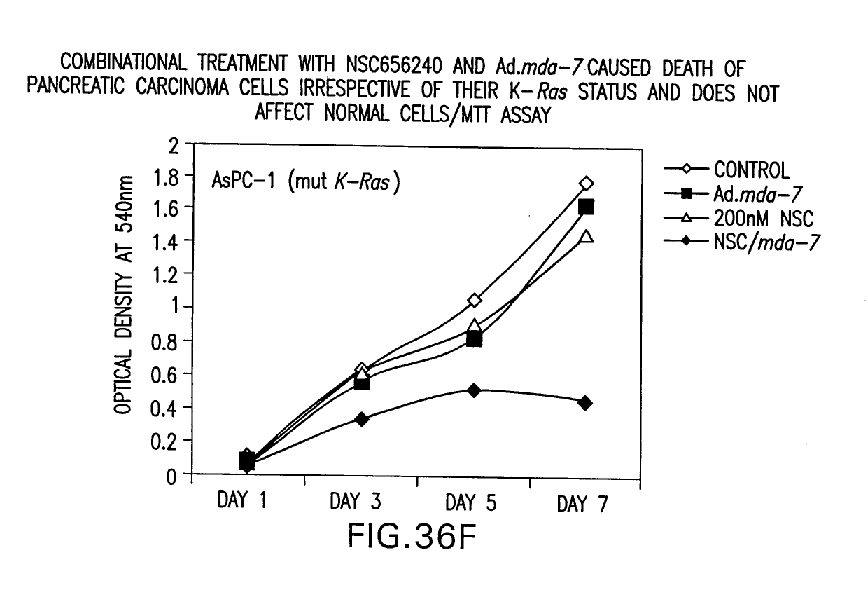

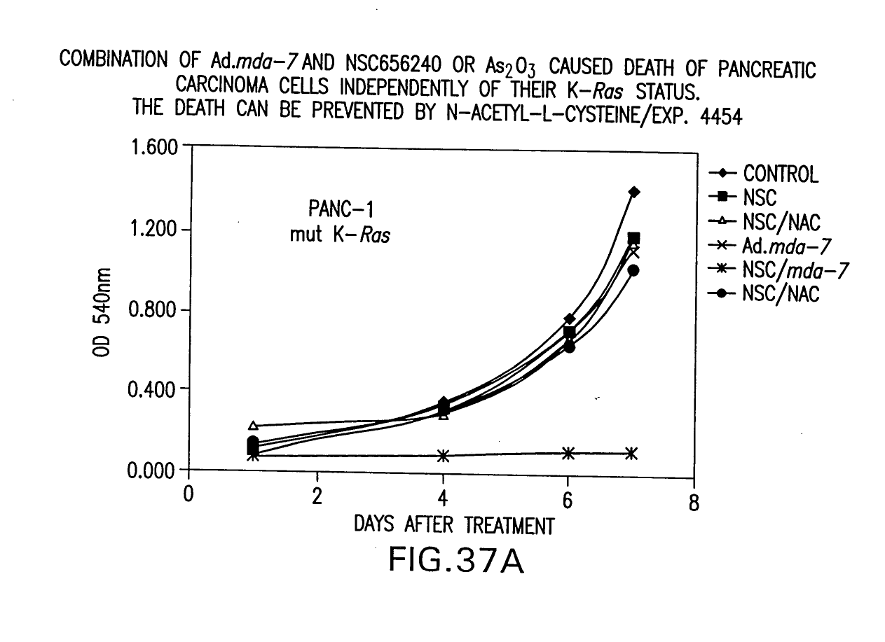

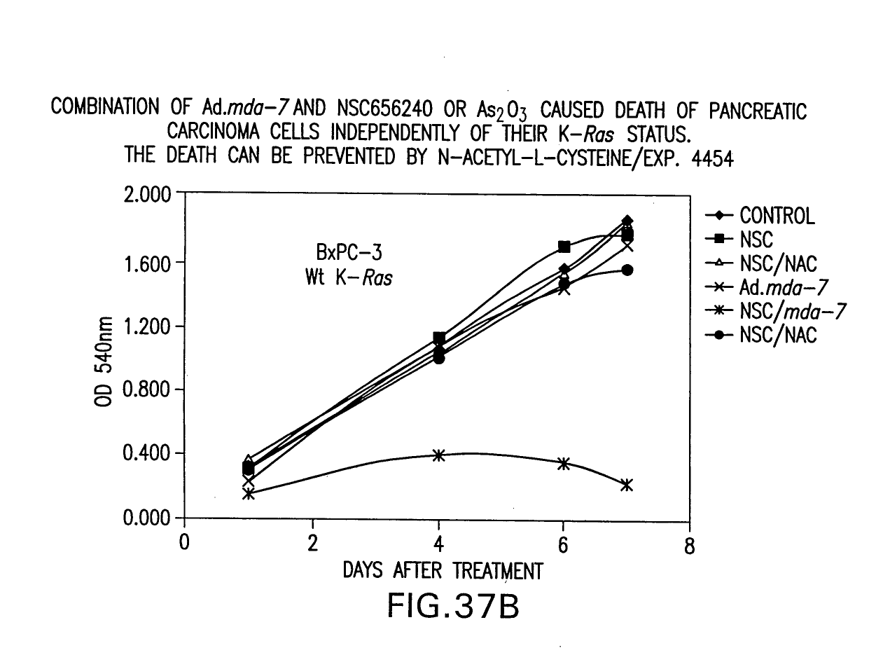

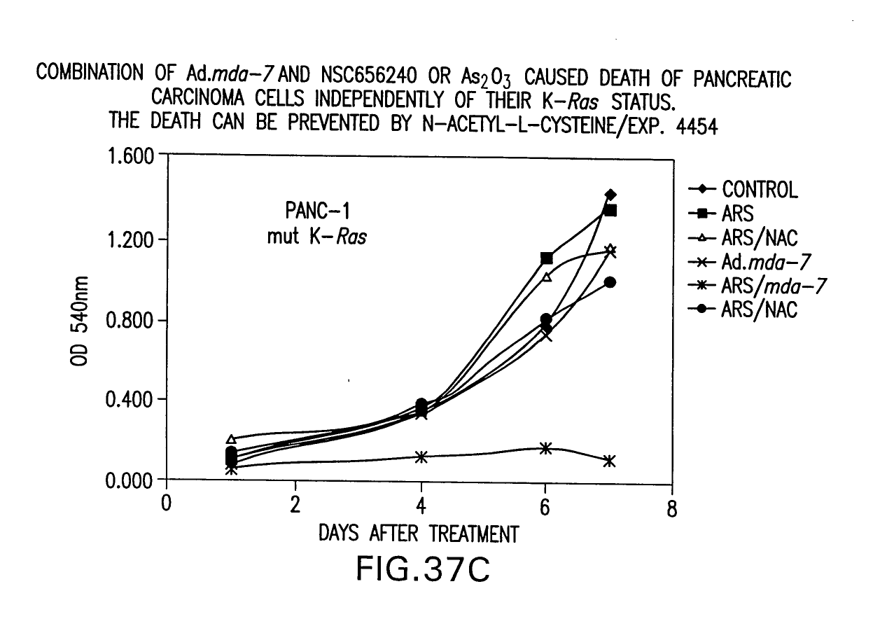

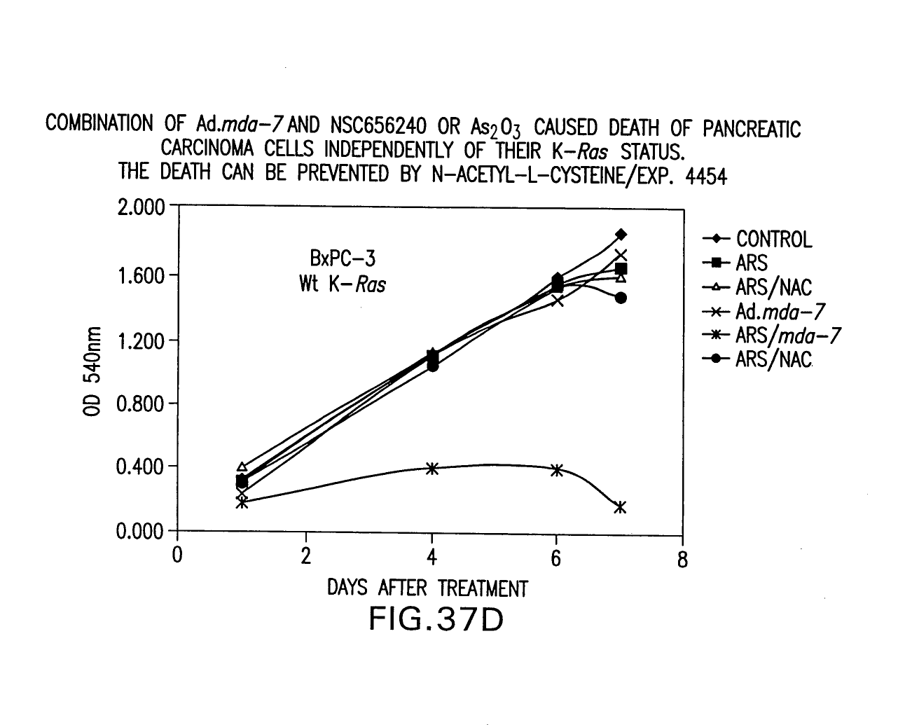

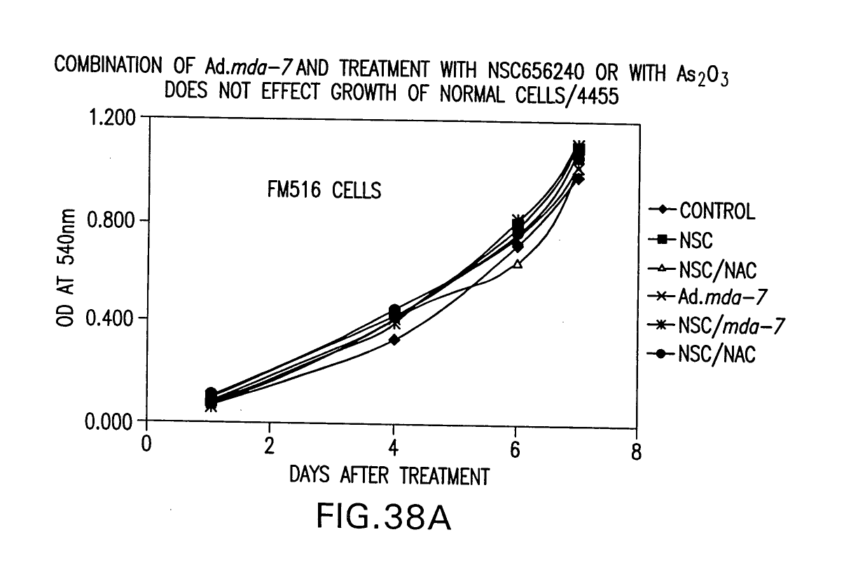

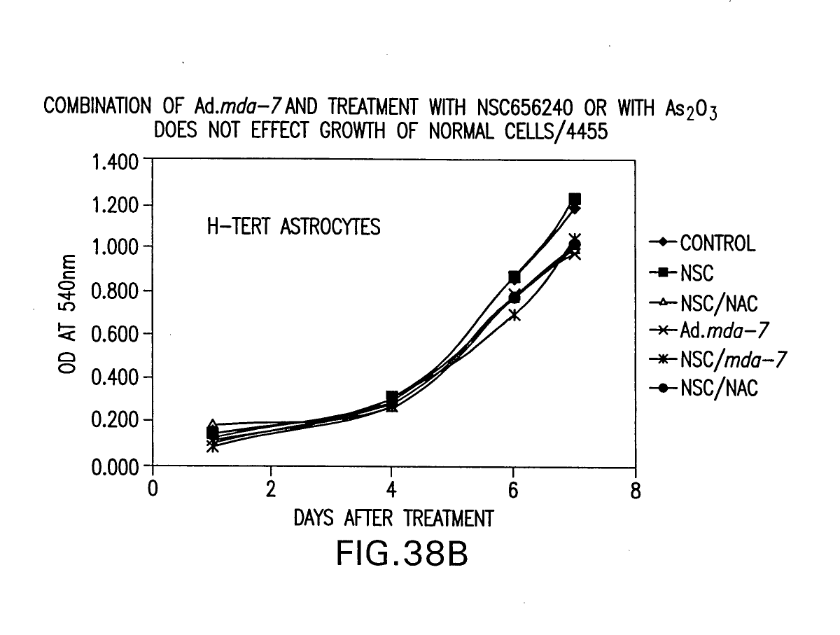

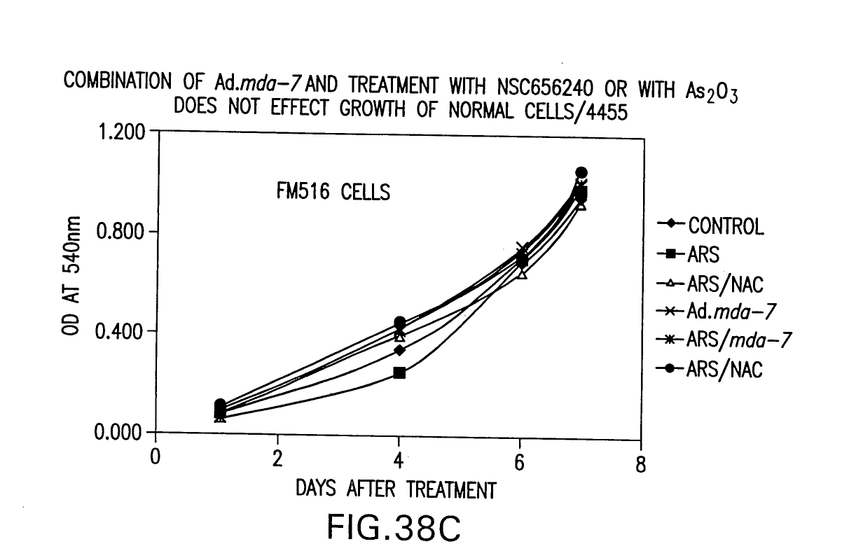

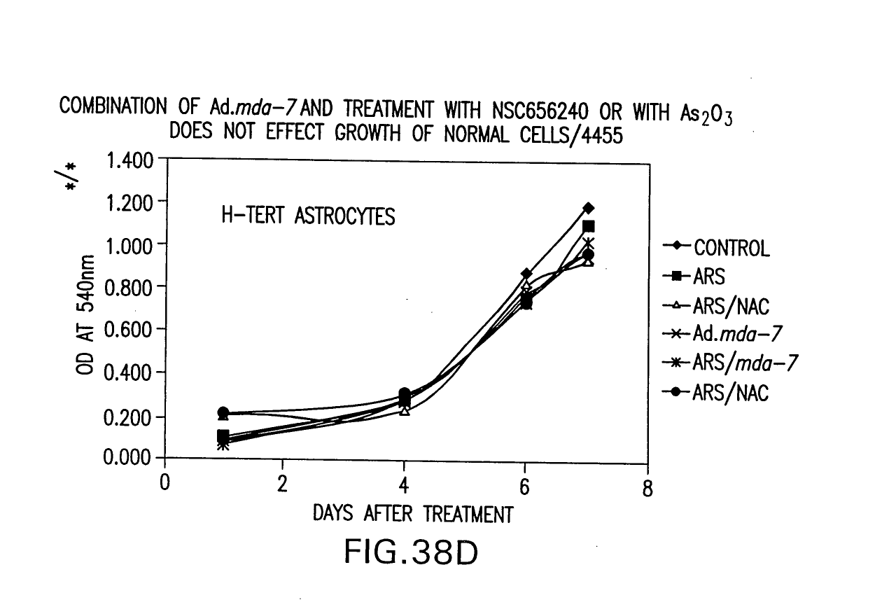

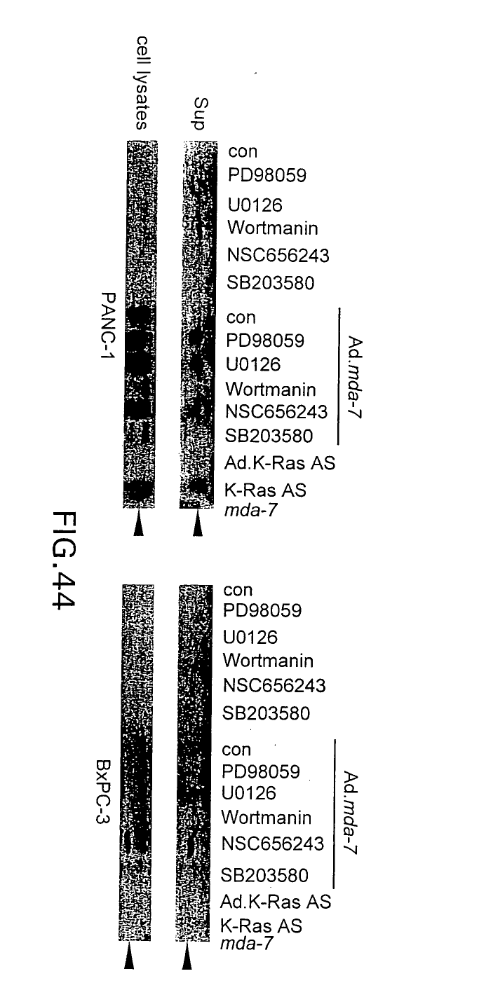

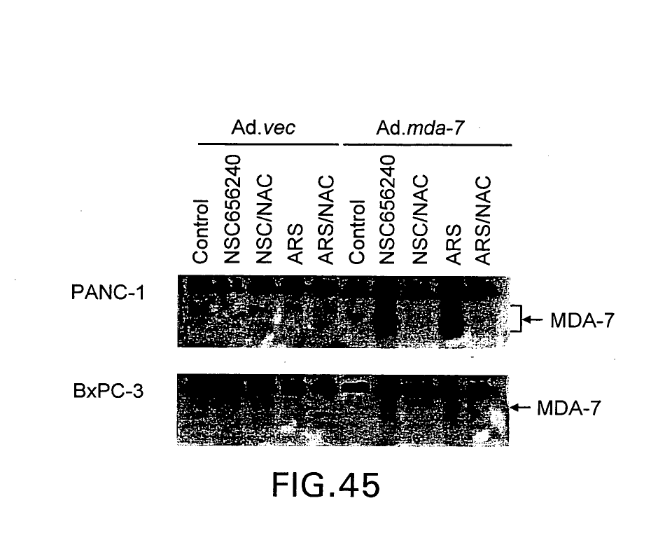

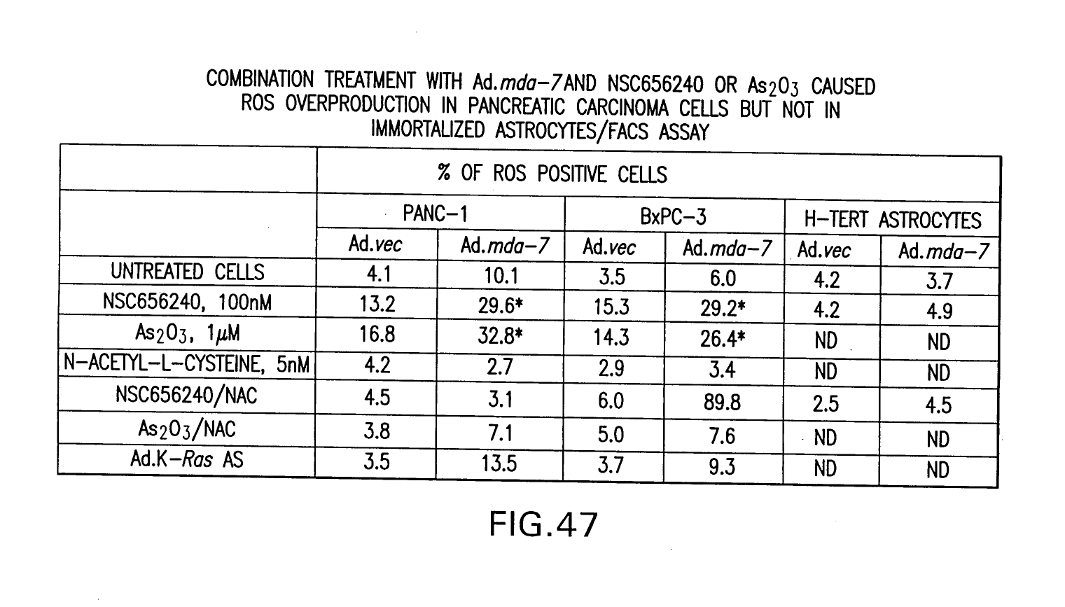

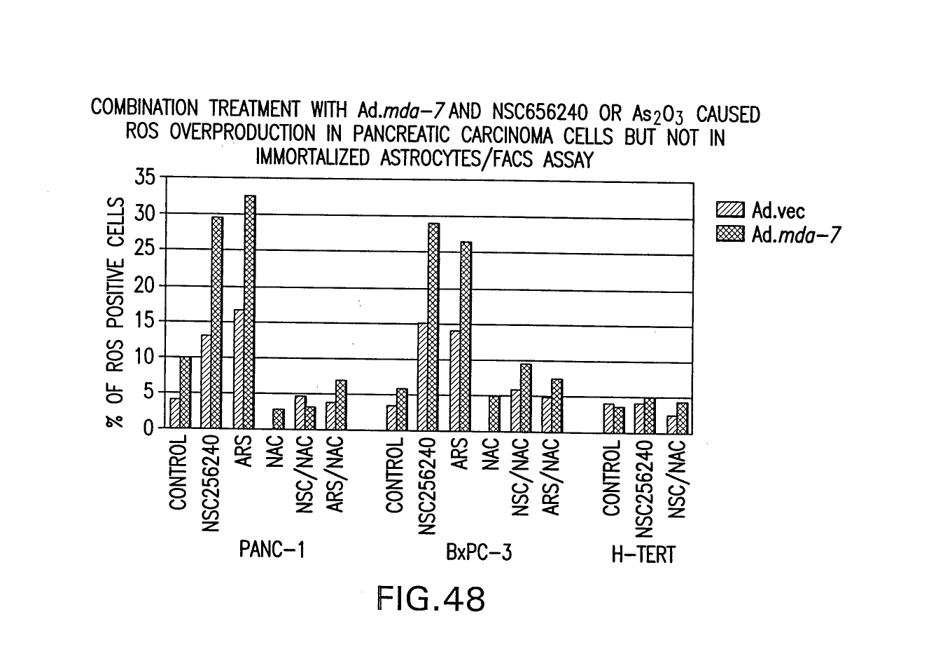

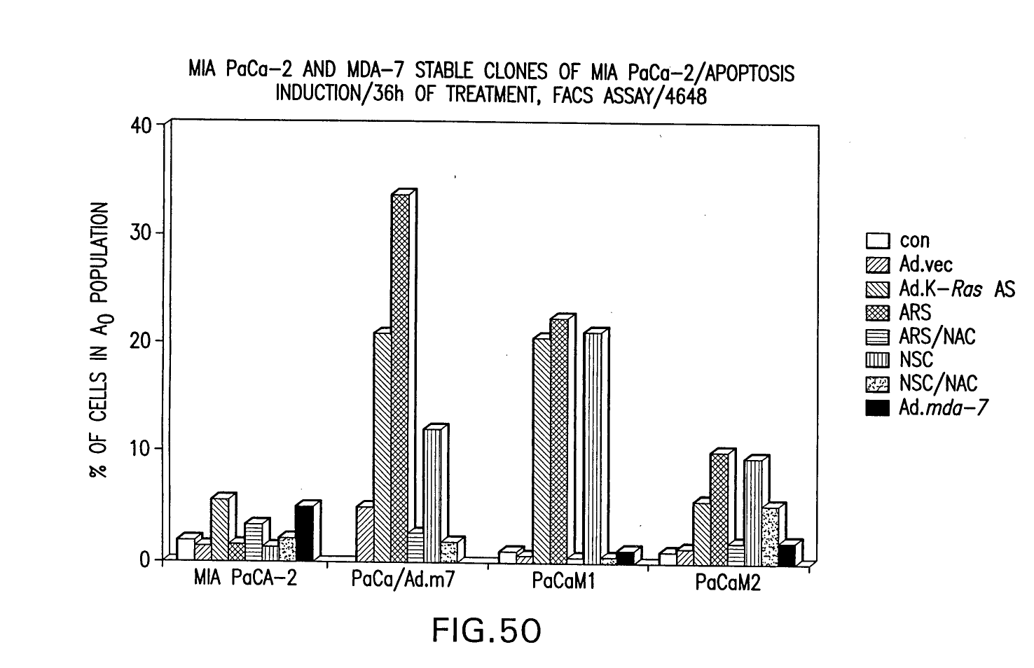

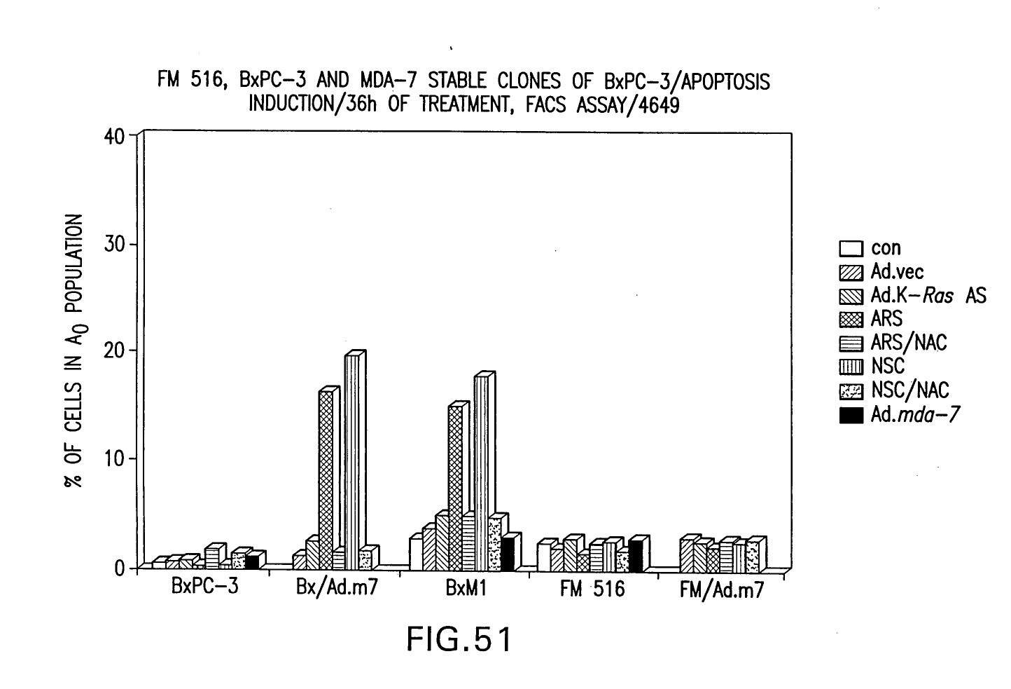

The results of the studies performed to evaluate the effects of MDA-7, alone or in combination with generators of free radicals are shown in FIGS. 36-51.

As shown in FIGS. 36-38, an MTT assay demonstrated that combinational treatment of pancreatic carcinoma cells with NSC656240 or As₂O₃ and Ad.mda-7 caused death of these cells irrespective of their K-ras status and does not affect growth of normal cells (immortalized human astrocytes or melanocytes). The fact that the death can be prevented by general antioxidant N-acetyl-cysteine indicates that oxidative processes are involved in mda-7 regulated pathway of killing.

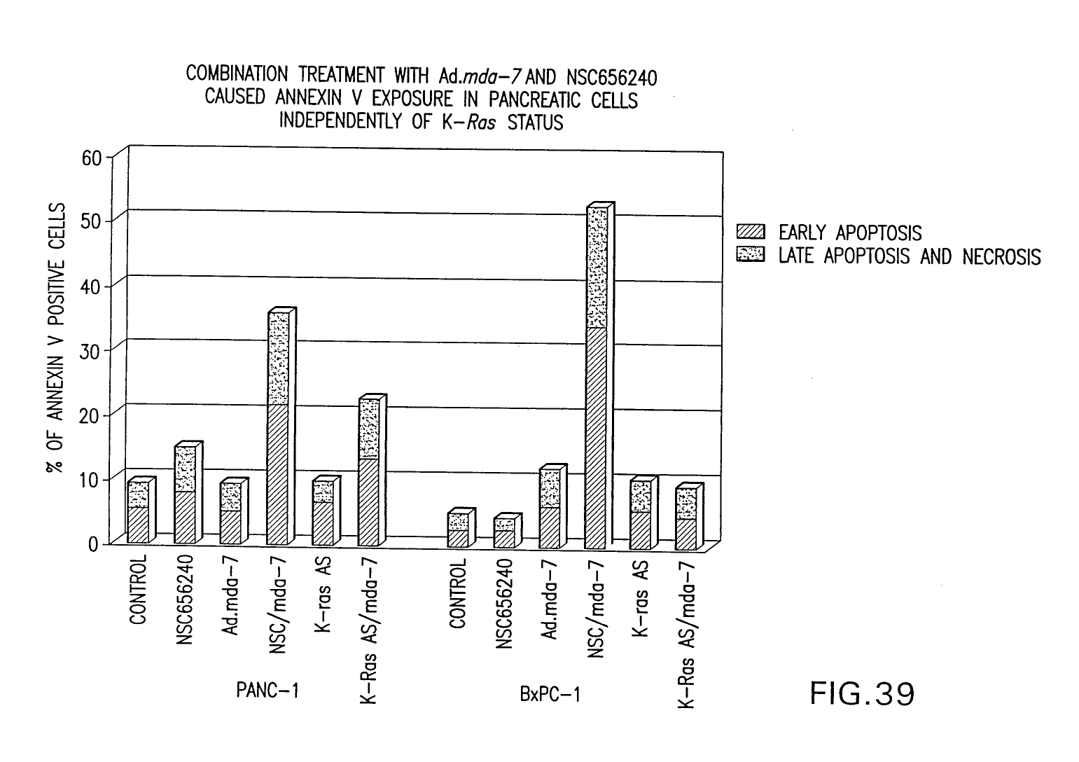

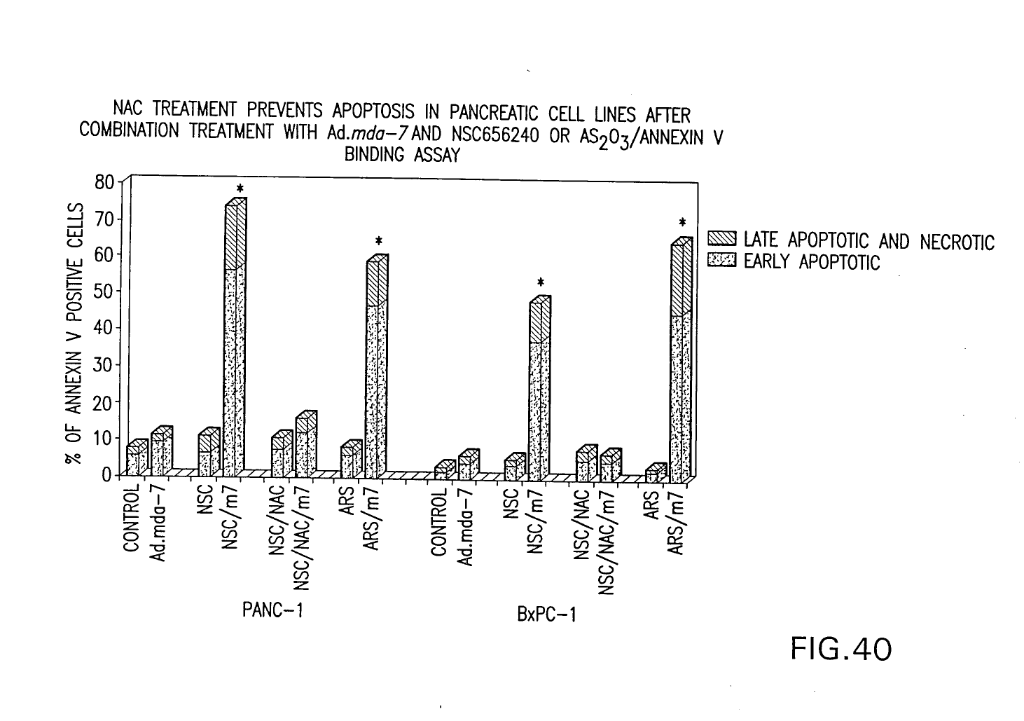

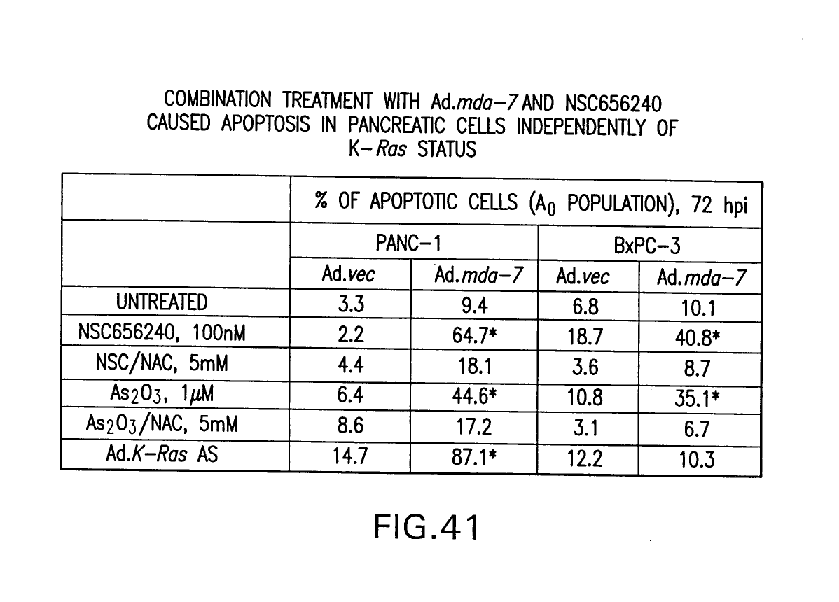

Combination treatment with Ad.mda-7 and NSC656240 (FIGS. 39 and 41) or As₂O₃ (FIGS. 40 and 41) caused apoptosis in pancreatic carcinoma cells independently of their K-ras status. Apoptosis was determined by annexin V exposure (early cytoplasmic apoptosis; FIGS. 39 and 40) and by determination of hypodiploid cells (A₀ population, late nuclear apoptosis; FIG. 41). NAC treatment prevents apoptosis in pancreatic cell lines after combination treatment with Ad.mda-7 and NSC656240 or As₂O₃ (FIGS. 39-41).

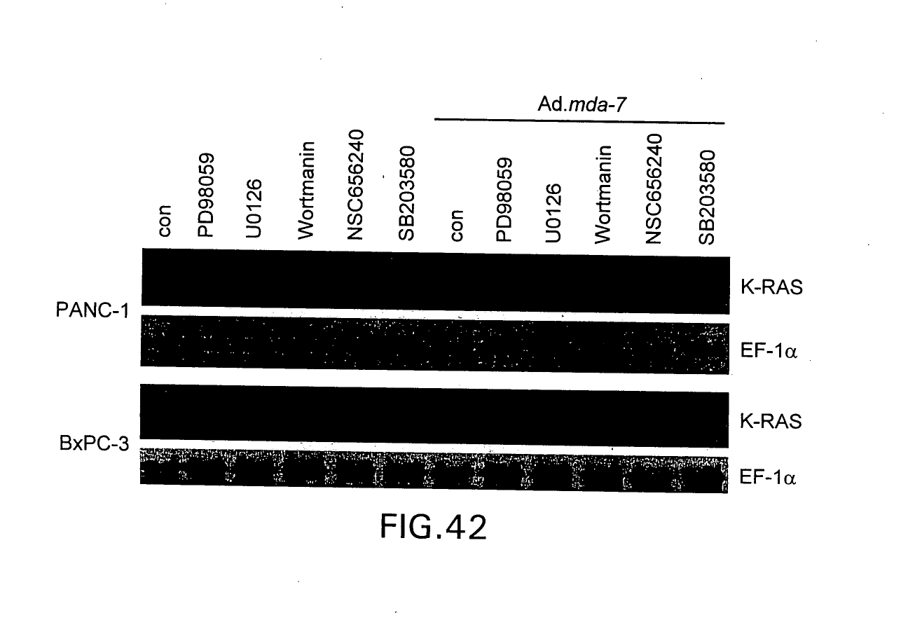

NSC656240 treatment, either alone or in combination with Ad.mda-7, does not down-regulate K-ras protein expression (FIG. 42).

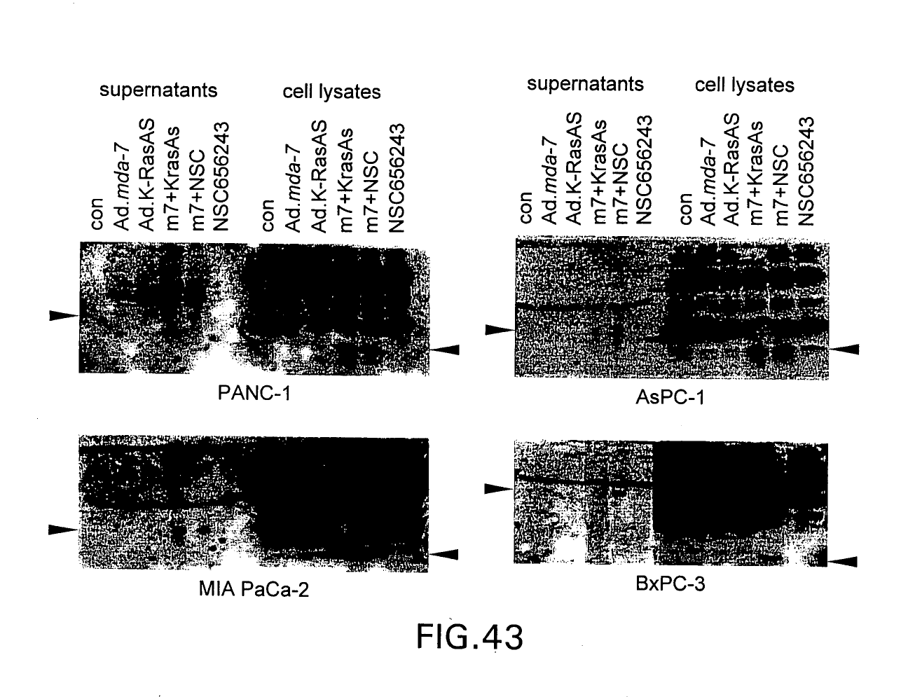

Combined treatment with NSC656240 or As₂O₃ with Ad.mda-7 leads to MDA-7 protein expression and secretion in wt and mutated K-ras pancreatic cancer cell lines (PANC-1, MIA PaCa-2 and AsPC-1), while Ad.mda-7 treatment in combination with Ad.K-rasAS, an adenovirus vector expressing an antisense oligonucleotide specific for mutant K-ras caused MDA-7 protein expression only in mut K-ras cell lines (BxPC-3). NAC treatment abrogates MDA-7 expression and secretion in both type of cells (FIGS. 43-45).

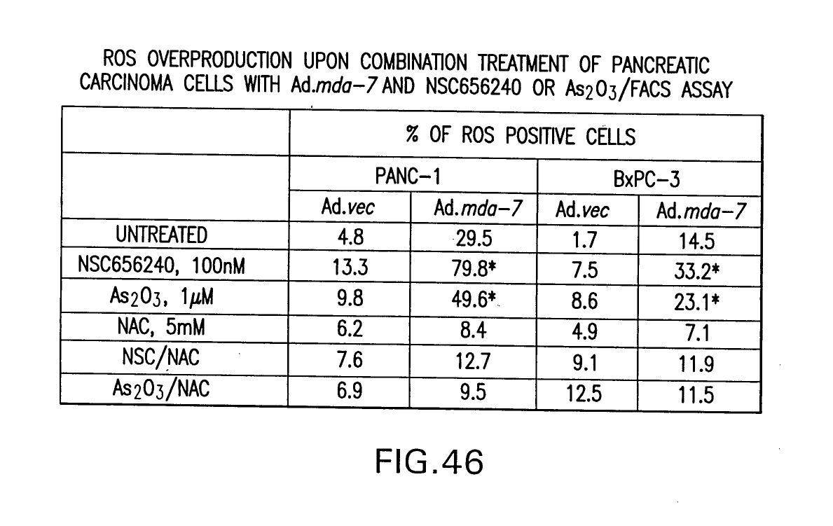

Combined treatment with Ad.mda-7 and NSC656240 or As₂O₃ Caused ROS overproduction in pancreatic carcinoma cells but not in immortalized astrocytes as demonstrated in FIGS. 46-48. NAC administration blocked ROS production.

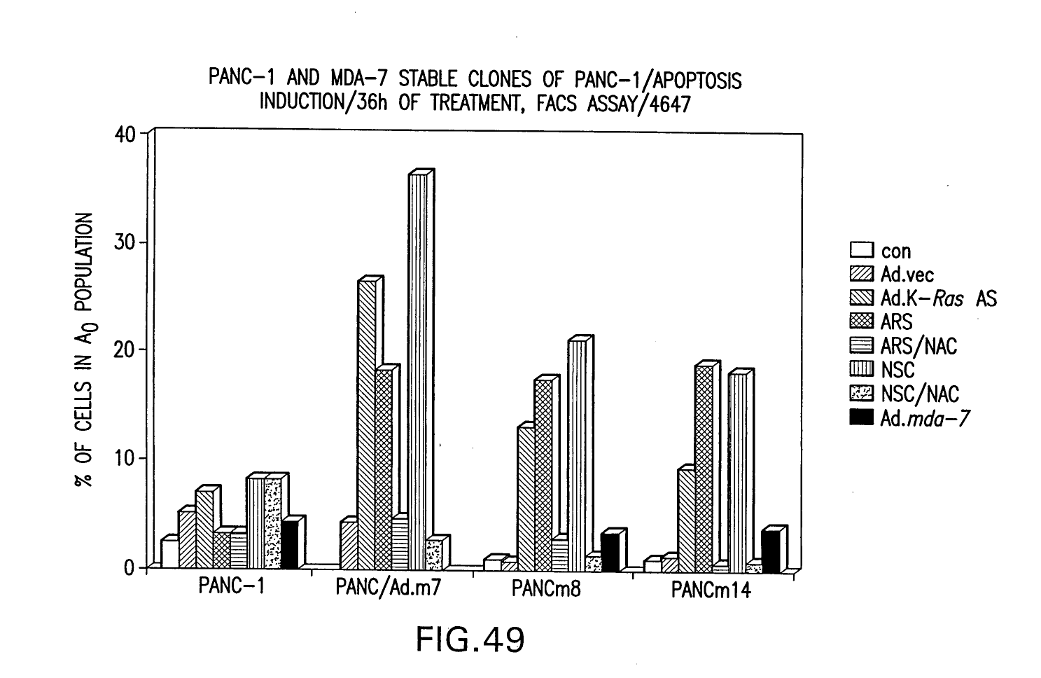

Treatment of pancreatic cancer cells stably transformed to express MDA-7 by NSC656240 or As₂O₃ caused apoptosis and cell death. NAC blocks these processes (FIGS. 49-51).

Various publications are cited herein, which are hereby incorporated by reference in their entireties.

What is claimed:

1. A method of treating a cancer in a subject comprising generating within one or more cancer cells of a subject an effective amount of MDA-7 and an effective amount of one or more free radicals, wherein generation within the one or more cancer cell of an effective amount of MDA-7 and an effective amount of one or more free radicals results in a reduced rate of growth or death of the cancer cell, and wherein the cancer to be treated is selected from the group consisting of melanoma, breast cancer, pancreatic cancer, prostate cancer, glioblastoma, Hodgkin's lymphoma, non-Hodgkins lymphoma, esophageal cancer, head and neck cancer, thyroid cancer, leukemia, cervical cancer, ovarian cancer, testicular cancer, gastric cancer, liver cancer, sarcoma, renal cancer, bladder cancer, neuroblastoma, osteosarcoma, renal cell carcinoma, retinoblastoma, colorectal cancer, hepatocellular carcinoma, multiple myeloma, nasopharyngeal cancer, progranulocytic leukemia, rhabdomyosarcoma, squamous cell carcinoma, and transitional cell carcinoma.

2. The method of claim 1, wherein the cancer to be treated is glioblastoma multiforme.

3. The method of claim 1, wherein the cancer to be treated is prostate cancer.

4. The method of claim 1, wherein the the cancer to be treated is renal cell carcinoma.

5. The method of claim 1, wherein the the cancer to be treated is ovarian cancer.

6. The method of claim 1, wherein the the cancer to be treated is melanoma.

7. The method of claim 1, wherein the the cancer to be treated is testicular cancer.

8. The method of claim 1, wherein the the cancer to be treated is pancreatic cancer.

9. The method of claim 1, wherein the the cancer to be treated is colorectal cancer.

10. The method of claim 1, wherein an effective amount of MDA-7 is generated within one or more cancer cells of a subject by introducing into one or more cells of the population an expressible form of the mda-7 gene.

11. The method of claim 10, wherein the mda-7 gene is a cDNA or a genomic DNA.

12. The method of claim 10, wherein the mda-7 gene comprises SEQ ID NO:1.

13. The method of claim 10, wherein the mda-7 gene encodes an MDA-7 protein.

14. The method of claim 13, wherein the MDA-7 protein comprises SEQ ID NO:2

15. The method of claim 13, wherein the MDA-7 protein is a fusion protein.

16. The method of claim 13, wherein the MDA-7 protein is secreted MDA-7.

17. The method of claim 10, wherein the mda-7 gene is comprised within a vector.

18. The method of claim 17, wherein the vector is selected from the group consisting of a viral vector and a non-viral vector.

19. The method of claim 18, wherein the viral vector is selected from the group consisting of an adenovirus vector, an adeno-associated virus vector, a retrovirus vector, a herpes virus vector, and a vaccinia virus vector.

20. A method of treating a cancer in a subject comprising generating within one or more cancer cells of a subject an effective amount of MDA-7 and an effective amount of one or more free radicals, wherein generation within the one or more cancer cell of an effective amount of MDA-7 and an effective amount of one or more free radicals results in a reduced rate of growth or death of the cancer cell, and wherein an effective amount of MDA-7 is generated within one or more cancer cells of a subject by administering to one or more cells of the population an MDA-7 protein.

21. The method of claim 20, wherein the MDA-7 protein comprises SEQ ID NO:2.

22. The method of claim 20, wherein the MDA-7 protein is a fusion protein.

23. The method of claim 20, wherein the MDA-7 protein is secreted MDA-7.

24. The method of claim 1, wherein wherein an effective amount of one or more free radicals is generated within a cancer cell of a subject by administration to the subject of ionizing radiation, a free radical, a generator of a free radical, a reactive oxygen species, a generator of a reactive oxygen species, or a disruptor of mitochondrial membrane potential.

25. The method of claim 24, wherein the generator of a free radical is selected from the group consisting of arsenic trioxide, NSC656240, N-(4-hydroxyphenyl) retinamide, and cisplatin.

26. The method of claim 24, wherein the reactive oxygen species is selected from the group consisting of singlet oxygen, hydrogen peroxide, superoxide anion, peroxynitrite, a hydroxyl radical, and an oxidant.

27. The method of claim 24, wherein the disruptor of mitochondrial membrane potential is PK 11195.

28. A method of inhibiting proliferation of a cancer cell in a subject comprising generating within one or more cancer cells of a subject an effective amount of MDA-7 and an effective amount of one or more free radicals, wherein generation within the one or more cancer cell of an effective amount of MDA-7 and an effective amount of one or more free radicals inhibits the proliferation of the cancer cell, and wherein the cancer to be treated is selected from the group consisting of melanoma, breast cancer, pancreatic cancer, prostate cancer, glioblastoma, Hodgkin's lymphoma, non-Hodgkins lymphoma, esophageal cancer, head and neck cancer, thyroid cancer, leukemia, cervical cancer, ovarian cancer, testicular cancer, gastric cancer, liver cancer, sarcoma, renal cancer, bladder cancer, neuroblastoma, osteosarcoma, renal cell carcinoma, retinoblastoma, colorectal cancer, hepatocellular carcinoma, multiple myeloma, nasopharyngeal cancer, progranulocytic leukemia, rhabdomyosarcoma, squamous cell carcinoma, and transitional cell carcinoma.

29. The method of claim 28, wherein the cancer to be treated is glioblastoma multiforme.

30. The method of claim 28, wherein the cancer to be treated is prostate cancer.

31. The method of claim 28, wherein the the cancer to be treated is renal cell carcinoma.

32. The method of claim 28, wherein the the cancer to be treated is ovarian cancer.

33. The method of claim 28, wherein the the cancer to be treated is melanoma.

34. The method of claim 28, wherein the the cancer to be treated is testicular cancer.

35. The method of claim 28, wherein the the cancer to be treated is pancreatic cancer.

36. The method of claim 28, wherein the the cancer to be treated is colorectal cancer.

37. The method of claim 28, wherein an effective amount of MDA-7 is generated within one or more cancer cells of a subject by introducing into one or more cells of the population an expressible form of the mda-7 gene.

38. The method of claim 37, wherein the mda-7 gene is a cDNA or a genomic DNA.

39. The method of claim 37, wherein the mda-7 gene comprises SEQ ID NO:1.

40. The method of claim 37, wherein the mda-7 gene encodes an MDA-7 protein.

41. The method of claim 40, wherein the MDA-7 protein comprises SEQ ID NO:2

42. The method of claim 40, wherein the MDA-7 protein is a fusion protein.

43. The method of claim 40, wherein the MDA-7 protein is secreted MDA-7.

44. The method of claim 37, wherein the mda-7 gene is comprised within a vector.

45. The method of claim 44, wherein the vector is selected from the group consisting of a viral vector and a non-viral vector.

46. The method of claim 45, wherein the viral vector is selected from the group consisting of an adenovirus vector, an adeno-associated virus vector, a retrovirus vector, a herpes virus vector, and a vaccinia virus vector.

47. A method of inhibiting proliferation of a cancer cell in a subject comprising generating within one or more cancer cells of a subject an effective amount of MDA-7 and an effective amount of one or more free radicals, wherein generation within the one or more cancer cell of an effective amount of MDA-7 and an effective amount of one or more free radicals inhibits the proliferation of the cancer cell, and wherein an effective amount of MDA-7 is generated within one or more cancer cells of a subject by administering to one or more cells of the population an MDA-7 protein.

48. The method of claim 47, wherein the MDA-7 protein comprises SEQ ID NO:2.

49. The method of claim 47, wherein the MDA-7 protein is a fusion protein.

50. The method of claim 47, wherein the MDA-7 protein is secreted MDA-7.

51. The method of claim 28, wherein wherein an effective amount of one or more free radicals is generated within a cancer cell of a subject by administration to the subject of ionizing radiation, a free radical, a generator of a free radical, a reactive oxygen species, a generator of a reactive oxygen species, or a disruptor of mitochondrial membrane potential.

52. The method of claim 51, wherein the generator of a free radical is selected from the group consisting of arsenic trioxide, NSC656240, N-(4-hydroxyphenyl) retinamide, and cisplatin.

53. The method of claim 51, wherein the reactive oxygen species is selected from the group consisting of singlet oxygen, hydrogen peroxide, superoxide anion, peroxynitrite, a hydroxyl radical, and an oxidant.

54. The method of claim 51, wherein the disrupter of mitochondrial membrane potential is PK 11195.

55. A method of promoting death of a cancer cell of a subject comprising generating within one or more cancer cells of a subject an effective amount of MDA-7 and an effective amount of one or more free radicals, wherein generation within the one or more cancer cell of an effective amount of MDA-7 and an effective amount of one or more free radicals promotes death of the cancer cell, and wherein the cancer to be treated is selected from the group consisting of melanoma, breast cancer, pancreatic cancer, prostate cancer, glioblastoma, Hodgkin's lymphoma, non-Hodgkins lymphoma, esophageal cancer, head and neck cancer, thyroid cancer, leukemia, cervical cancer, ovarian cancer, testicular cancer, gastric cancer, liver cancer, sarcoma, renal cancer, bladder cancer, neuroblastoma, osteosarcoma, renal cell carcinoma, retinoblastoma, colorectal cancer, hepatocellular carcinoma, multiple myeloma, nasopharyngeal cancer, progranulocytic leukemia, rhabdomyosarcoma, squamous cell carcinoma, and transitional cell carcinoma.

56. The method of claim 55, wherein the cancer to be treated is glioblastoma multiforme.

57. The method of claim 55, wherein the cancer to be treated is prostate cancer.

58. The method of claim 55, wherein the the cancer to be treated is renal cell carcinoma.

59. The method of claim 55, wherein the the cancer to be treated is ovarian cancer.

60. The method of claim 55, wherein the the cancer to be treated is melanoma.

61. The method of claim 55, wherein the the cancer to be treated is testicular cancer.

62. The method of claim 55, wherein the the cancer to be treated is pancreatic cancer.

63. The method of claim 55, wherein the the cancer to be treated is colorectal cancer.

64. The method of claim 55, wherein an effective amount of MDA-7 is generated within one or more cancer cells of a subject by introducing into one or more cells of the population an expressible form of the mda-7 gene.

65. The method of claim 64, wherein the mda-7 gene is a cDNA or a genomic DNA.

66. The method of claim 64, wherein the mda-7 gene comprises SEQ ID NO:1.

67. The method of claim 64, wherein the mda-7 gene encodes an MDA-7 protein.

68. The method of claim 67, wherein the MDA-7 protein comprises SEQ ID NO:2

69. The method of claim 67, wherein the MDA-7 protein is a fusion protein.

70. The method of claim 67, wherein the MDA-7 protein is secreted MDA-7.

71. The method of claim 64, wherein the mda-7 gene is comprised within a vector.

72. The method of claim 71, wherein the vector is selected from the group consisting of a viral vector and a non-viral vector.

73. The method of claim 72, wherein the viral vector is selected from the group consisting of an adenovirus vector, an adeno-associated virus vector, a retrovirus vector, a herpes virus vector, and a vaccinia virus vector.

74. A method of promoting death of a cancer cell of a subject comprising generating within one or more cancer cells of a subject an effective amount of MDA-7 and an effective amount of one or more free radicals, wherein generation within the one or more cancer cell of an effective amount of MDA-7 and an effective amount of one or more free radicals promotes death of the cancer cell, and wherein an effective amount of MDA-7 is generated within one or more cancer cells of a subject by administering to one or more cells of the population an MDA-7 protein.

75. The method of claim 74, wherein the MDA-7 protein comprises SEQ ID NO:2.

76. The method of claim 74, wherein the MDA-7 protein is a fusion protein.

77. The method of claim 74, wherein the MDA-7 protein is secreted MDA-7.

78. The method of claim 55, wherein wherein an effective amount of one or more free radicals is generated within a cancer cell of a subject by administration to the subject of ionizing radiation, a free radical, a generator of a free radical, a reactive oxygen species, a generator of a reactive oxygen species, or a disruptor of mitochondrial membrane potential.

79. The method of claim 78, wherein the generator of a free radical is selected from the group consisting of arsenic trioxide, NSC656240, N-(4-hydroxyphenyl) retinamide, and cisplatin.

80. The method of claim 78, wherein the reactive oxygen species is selected from the group consisting of singlet oxygen, hydrogen peroxide, superoxide anion, peroxynitrite, a hydroxyl radical, and an oxidant.

81. The method of claim 78, wherein the disrupter of mitochondrial membrane potential is PK 11195.
